(19) United States
(12) Patent Application Publication
(10) Pub. No.: US 2025/0164491 A1
(43) Pub. Date: May 22, 2025

(54) Methods of Identifying and Treating Progressive Colorectal Cancer

(71) Applicant: President and Fellows of Harvard College, Cambridge, MA (US)

(72) Inventors: Peter K. Sorger, Cambridge, MA (US); Sandro Santagata, Cambridge, MA (US); Jia-Ren Lin, Cambridge, MA (US); Yu-An Chen, Cambridge, MA (US)

(21) Appl. No.: 18/897,162

(22) Filed: Sep. 26, 2024

Related U.S. Application Data
(60) Provisional application No. 63/541,174, filed on Sep. 28, 2023.

(51) Int. Cl. G01N 33/574; G01N 33/58

(52) U.S. Cl. CPC G01N 33/57419; G01N 33/582; G01N 2333/4742; G01N 2333/70596

(57) ABSTRACT

Methods are provided for identifying progressive colorectal cancer within an individual, and to methods of treating progressive colorectal cancer.

### RELATED U.S. APPLICATIONS

This application claims the benefit of the filing date of U.S. Provisional Patent Application No. 63/541,174, filed on Sep. 28, 2023.

### STATEMENT OF GOVERNMENT INTERESTS

This invention was made with government support under CA233262 awarded by National Institutes of Health (NIH). The government has certain rights in the invention

### TECHNICAL FIELD

The present disclosure relates to methods for distinguishing aggressive versus nonaggressive forms of colorectal cancer in an individual, and to methods of treatment of such aggressive or nonaggressive colorectal cancer.

### DESCRIPTION OF THE RELATED ART

The microanatomy of fixed and stained tissues has been studied using light microscopy for over two centuries, see e.g., Bock, O. A history of the development of histology up to the end of the nineteenth century. Research (2015); Paget, S. THE DISTRIBUTION OF SECONDARY GROWTHS IN CANCER OF THE BREAST. The Lancet 133, 571-573 (1889), and immunohistochemistry (IHC) has been in widespread use for 50 years, see e.g., Coons, A. H., Creech, H. J. & Jones, R. N. Immunological Properties of an Antibody Containing a Fluorescent Group. Proceedings of the Society for Experimental Biology and Medicine 47, 200-202 (1941). Histopathology review of hematoxylin and eosin (H&E) stained tissue sections, complemented by immunohistochemistry (IHC) and exome sequencing, remains the primary approach for diagnosing and managing many diseases, particularly cancer, see e.g., Robbins & Cotran Pathologic Basis of Disease—9780323531139. US Elsevier Health world wide website us.elsevierhealth.com/robbins-cotran-pathologic-basis-of-disease—9780323531139.html. More recently, a range of computational methods have been developed to automatically extract information from H&E images, see e.g., Demir, C. & Yener, B. Automated cancer diagnosis based on histopathological images: A systematic survey. (2004), and the use of machine learning and artificial intelligence approaches (ML/AI) is leading to rapid progress in computer-assisted diagnosis, see e.g., Cui, M. & Zhang, D. Y. Artificial intelligence and computational pathology. Lab Invest 101, 412-422 (2021). However, the images in current digital pathology systems-acquired from conventional histology and IHC methods-generally lack the molecular precision and depth of quantitative analysis needed to optimally predict outcomes, guide the selection of targeted therapies, and enable research into the molecular mechanisms of disease, see e.g., Wharton, K. A. et al. Tissue Multiplex Analyte Detection in Anatomic Pathology—Pathways to Clinical Implementation. Frontiers in Molecular Biosciences 8, (2021).

The transition from H&E-based histopathology to digital technologies, see e.g., Abels, E. et al. Computational pathology definitions, best practices, and recommendations for regulatory guidance: a white paper from the Digital Pathology Association. J. Pathol. 249, 286-294 (2019), is occurring concurrently with the introduction of methods for obtaining 10-80-plex data from fixed tissue sections (e.g., MxIF, CyCIF, CODEX, 4i, mIHC, MIBI, IBEX, and IMC, see e.g., Angelo, M. et al. Multiplexed ion beam imaging of human breast tumors. Nat. Med. 20, 436-442 (2014); Gerdes, M. J. et al. Highly multiplexed single-cell analysis of formalin-fixed, paraffin-embedded cancer tissue. Proc. Natl. Acad. Sci. U.S.A. 110, 11982-11987 (2013); Giesen, C. et al. Highly multiplexed imaging of tumor tissues with subcellular resolution by mass cytometry. Nat. Methods 11, 417-422 (2014); Goltsev, Y. et al. Deep Profiling of Mouse Splenic Architecture with CODEX Multiplexed Imaging. Cell 174, 968-981.e15 (2018); Gut, G., Herrmann, M. D. & Pelkmans, L. Multiplexed protein maps link subcellular organization to cellular states. Science 361, (2018); Tsujikawa, T. et al. Quantitative Multiplex Immunohistochemistry Reveals Myeloid-Inflamed Tumor-Immune Complexity Associated with Poor Prognosis. Cell Rep 19, 203-217 (2017); Lin, J. R. et al. Highly multiplexed immunofluorescence imaging of human tissues and tumors using t-CyCIF and conventional optical microscopes. Elife 7, e31657 (2018). These high-plex imaging methods enable deep morphological and molecular analysis of normal and diseased tissues from humans and animal models, see e.g., Goltsev, Y. et al. Deep Profiling of Mouse Splenic Architecture with CODEX Multiplexed Imaging. Cell 174, 968-981.e15 (2018); Färkkilä, A. et al. Immunogenomic profiling determines responses to combined PARP and PD-1 inhibition in ovarian cancer. Nat Commun 11, 1459 (2020); Launonen, I. M. et al. Single-cell tumor-immune microenvironment of BRCA1/2 mutated highgrade serous ovarian cancer. Nat Commun 13, 835 (2022); Schürch, C. M. et al. Coordinated Cellular Neighborhoods Orchestrate Antitumoral Immunity at the Colorectal Cancer Invasive Front. Cell 182, 1341-1359.e19 (2020); Wagner, J. et al. A Single-Cell Atlas of the Tumor and Immune Ecosystem of Human Breast Cancer. Cell 177, 1330-1345.e18 (2019), and generate spatially resolved information that is an ideal complement to other single cell methods, such as scRNA sequencing. Whereas some imaging methods require frozen samples, those that are compatible with formaldehyde-fixed and paraffin-embedded (FFPE) specimens—the type of specimens universally acquired for diagnostic purposes—make it possible to tap into large archives of human biopsy and resection specimens, see e.g., Burger, M. L. et al. Antigen dominance hierarchies shape TCF1+ progenitor CD8 T cell phenotypes in tumors. Cell 184, 4996-5014.e26 (2021); Gaglia, G. et al. Temporal and spatial topography of cell proliferation in cancer. Nat Cell Biol 24, 316-326 (2022). Moreover, whereas many high-plex imaging studies involve tissue microarrays (TMA; arrays of many 0.3 to 1 mm specimens on a single slide) or the small fields of view characteristic of mass-spectrometry based imaging, see e.g., Angelo, M. et al. Multiplexed ion beam imaging of human breast tumors. Nat. Med. 20, 436-442 (2014); Giesen, C. et al. Highly multiplexed imaging of tumor tissues with subcellular resolution by mass cytometry. Nat. Methods 11, 417-422 (2014), whole-slide imaging is required for clinical research and practice both to achieve sufficient statistical power, see e.g., Lin, J. R. et al. Multiplexed 3D atlas of state transitions and immune interactions in colorectal cancer. bioRxiv 2021.03.31.437984 (2021) doi: 10.1101/2021.03.31.437984, and as an FDA requirement, see e.g., Health, C. for D. and R. Technical Performance Assessment of Digital Pathology Whole Slide Imaging Devices. U.S. Food and Drug Administration http://www.fda.gov/regulatoryinformation/search-fda-guidance-documents/technical-performance-assessment-digital-pathologywhole-slide-imaging-devices (2019).

Histopathology review of H&E images, a top-down approach, exploits prior knowledge about the cellular and acellular structures and morphologies associated with disease to analyze images, see e.g., Weiser, M. R. AJCC 8th Edition: Colorectal Cancer. Ann Surg Oncol 25, 1454-1455 (2018). In contrast, research using highly multiplexed imaging most commonly relies on a bottom-up approach in which cell types are enumerated and neighborhoods associated with disease are identified computationally, see e.g., Angelo, M. et al. Multiplexed ion beam imaging of human breast tumors. Nat. Med. 20, 436-442 (2014); Giesen, C. et al. Highly multiplexed imaging of tumor tissues with subcellular resolution by mass cytometry. Nat. Methods 11, 417-422 (2014). Accordingly, a need exists to combine these approaches in research and diagnostic settings, thereby combining standard clinical practice with single cell analysis of the tumor microenvironment to identify prognostic markers of cancer, such as colorectal cancer.

### SUMMARY OF THE INVENTION

Aspects of the present disclosure are directed to improving treatment of patients with colorectal cancer through the discovery that a colorectal cancer tissue sample can be distinguished between progressive colorectal cancer and colorectal cancer characterized by progression free survival using the analysis methods described herein. For purposes of the present disclosure “progressive colorectal cancer” is defined as is known in the art by an objective measurement that a colorectal cancer tumor has grown in size, as commonly measured radiologically or by objective evidence of tumor recurrence at the same primary site or spread of the tumor at a metastatic site. Progressing colorectal cancer may be characterized as an increase in the size of pre-existing tumors or the appearance of new tumors. See Eisenhauer E A, Therasse P, Bogaerts J, Schwartz L H, Sargent D, Ford R, Dancey J, Arbuck S, Gwyther S, Mooney M, Rubinstein L, Shankar L, Dodd L, Kaplan R, Lacombe D, Verweij J. New response evaluation criteria in solid tumours: revised RECIST guideline (version 1.1). Eur J Cancer. 2009; 45:228-47. According to one exemplary aspect, “progressive colorectal cancer” may be characterized by a 20% growth in the size of the tumor or objective evidence of tumor recurrence at the same primary site or spread of the tumor at a metastatic site. See for example Oxnard G, Morris J, Hodi F, et al. When Progressive Disease Does Not Mean Treatment Failure: Reconsidering the Criteria for Progression. Journal of the National Cancer Institute. 2012. 140 (26): 1534-1541. doi: 10.1093/jnci/djs353. “Progression” may be characterized by imaging techniques (plain radiograms, CT scans, MRI, PET scans, ultrasounds) or other aspects. “Progression” may also be characterized by biochemical aspects, such as an increase in a tumor marker. According to one aspect, radiological aspects of a colorectal tumor may be defined according to RECIST criteria, as is known in the art. Progression may also be characterized due to the appearance of a new lesion or to unequivocal progression in other lesions, such as an increase in size or the lesions spreading to nearby tissues.

For purposes of the present disclosure “nonprogressive colorectal cancer” or “stable colorectal cancer” or colorectal cancer characterized under a “progression free survival” condition is defined as is known in the art by a lack of tumor growth or the tumor being characterized as being stable or unchanging or not progressing or tumor growth which is below that characterized as “progressive.” According to one exemplary aspect, “progression-free survival” as is known in the art is the length of time during and after the treatment of a disease, such as cancer, that a patient lives with the disease, but it does not get worse.

Accordingly, methods are provided for distinguishing between progressive colorectal cancer and nonprogressive or stable colorectal cancer such as is characterized under progression free survival condition by (1) quantifying amounts of a marker or markers, one or more markers, two or more markers or a plurality of markers, such as pan-cytokeratin and E-cadherin, in tumor cells of a colorectal cancer tissue sample, (2) quantifying amounts of the marker or markers in nontumor cells of the colorectal cancer tissue sample, and (3) comparing the amounts of the marker or markers in tumor cells relative to the amounts of the marker or markers in nontumor cells, wherein a lower amount of a designated marker, such as E-cadherin, in the tumor cells indicates that the tumor cells are progressive colorectal cancer cells.

In another aspect, the ratio of the amounts of two exemplary markers, such as pan-cytokeratin and E-cadherin, in nontumor cells of a cancer tissue sample, such as a colorectal cancer tissue sample, is determined as a baseline or control value or a value against which another determined ratio of the amounts of the two exemplary markers in tumor cells of the cancer tissue sample, such as a colorectal cancer tissue sample, can be normalized. The ratios can be compared and how the ratio of the markers in the tumor cells differs from the ratio of the markers in the nontumor cells provides a basis for determining whether the tumor cells are progressive cancer tumor cells or are nonprogressive or stable cancer tumor cells. If the ratio of the markers in the tumor cells differs from the ratio of the markers in the nontumor cells, then the tumor cells are progressive cancer tumor cells. If the ratio of the markers in the tumor cells is substantially similar to the ratio of the markers in the nontumor cells, then the tumor cells are nonprogressive or stable cancer tumor cells. Nonprogressive cancer tumor cells are correlated with progression free survival of a patient and can be used to determine treatment for the patient. Likewise, a determination of progressive cancer tumor cells can be used to determine treatment for the patient. Based on the methods described herein of distinguishing a colorectal cancer tissue sample as being a progressive form of colorectal cancer or is characterized as nonprogressive or stable colorectal cancer such as is characterized under progression free survival conditions, the patient can be treated or have treatment adjusted based on either having a progressive form of colorectal cancer or a form of nonprogressive or stable colorectal cancer. The improved methods advantageously provide a patient with colorectal cancer with a treatment option or altering a treatment option if confirmed as having nonprogressive or stable colorectal cancer.

It is to be understood that the amounts of the two markers can be used to determine a ratio and it is not critical that any particular marker be the dividend or the divisor, so long as the two markers are consistently used as the dividend and divisor when determining the ratio of the markers in the tumor cells and the nontumor cells. For example, the methods described herein contemplate being able to differentiate between progressive tumor cells and nonprogressive tumor cells using a ratio of Marker1/Marker2 for the tumor cells and a ratio of Marker1/Marker2 for the nontumor cells and comparing to see how the ratios differ. The same differentiation between progressive tumor cells and nonprogressive tumor cells can be achieved using a ratio of Marker2/Marker1 for the tumor cells and a ratio of Marker2/Marker1 for the nontumor cells and comparing to see how the ratios differ.

The present disclosure provides methods of analyzing a colorectal cancer tissue sample using an immunofluorescence assay to characterize the amount of markers pan-cytokeratin and E-cadherin in tumor cells versus nontumor cells of a colorectal tissue sample. According to one aspect, the same tissue sample is stained using dyes, such as H&E and eosin, to identify tissue morphology including the tumor micro environment. The relationship between the markers pan-cytokeratin and E-cadherin and the tumor microenvironment versus the tumor margin or invasive margin may be determined and compared.

According to one aspect, markers capable of differentiating between progressive cancer and nonprogressive cancer can be determined using an immunofluorescence assay, a tissue staining assay and computer models generated by machine learning or artificial intelligence algorithms or programs. The methods can identify topics of tissue within a whole slide tissue sample including both tumor cells and nontumor cells, and amounts of markers correlating with progressive cancer or nonprogressive or stable cancer, such as that associated with progression free survival.

Accordingly, aspects of the present disclosure are directed to methods for identifying whether an individual confirmed or suspected of having colorectal cancer has a progressive form of colorectal cancer or is characterized as a cancer patient with nonprogressive or stable colorectal cancer. According to one aspect, colorectal cancer tissue from a patient is obtained and analyzed to determine whether the colorectal cancer is a progressive colorectal cancer or whether the colorectal cancer is nonprogressive or stable. Based on a determination of whether the colorectal cancer is progressive or nonprogressive or stable, the patient can receive treatment, reduce treatment, stop treatment or not begin treatment. For example, if the colorectal cancer is progressive, traditional treatment for colorectal cancer, such as surgery, chemotherapy, radiation therapy or immunotherapy may be applied. If the colorectal cancer is characterized as nonprogressive or stable, such as under a progression free survival condition, the patient may opt for reduced treatment from traditional treatment being applied or may opt for stopped treatment or no treatment, since odds of survival are increased for colorectal cancer characterized under a progression free survival condition versus a progressive form of colorectal cancer.

According to one aspect, colorectal cancer tissue from a patient is analyzed to determine whether the tumor marker pan-cytokeratin and the cohesion protein E-cadherin are present in cells in the tissue sample including tumor cells and nontumor cells. According to one aspect, the tissue sample is indicative of progressive cancer where there is a high probability that cells with a high level of pan-cytokeratin (pCK^{high}) and cells with a low level of E-cadherin (E-cadherin^{low}) are found in proximity to each other.

The identification of the combination of pan-cytokeratin and E-cadherin as markers differentiating between progressive colorectal cancer and nonprogressive or stable colorectal cancer, included combining histopathology using stained tissue, such as Hematoxylin and Eosin (H&E) stained tissue, with immunofluorescence images of a whole slide format of the same tissue and cells contacted with detectable antibodies for pan-cytokeratin and E-cadherin, and generating computational models based on immune filtration or tumor intrinsic features that are highly predictive of a cancer under a progression free survival condition. The methods of identifying markers as described herein are based on visualizing cells in a whole tissue sample to combine molecular data with tissue structure and generate computer modelling to determine whether colorectal cancer tissue is progressive or is nonprogressive or stable. The methods described herein are used to distinguish between whether a patient has progressive colorectal cancer or whether the patient has nonprogressive or stable colorectal cancer. Based on this information, if the patient is indicated to have progressive colorectal cancer, the patient may be treated for progressive colorectal cancer using methods known to those of skill in the art. Based on this information, if the patient is indicated to have nonprogressive or stable colorectal cancer, the treatment being received by the patient may be altered, reduced or eliminated or the patient may not be treated for the colorectal cancer.

According to methods described later herein, whole-slide immunofluorescence (IF) imaging for spatial biomarkers is followed by H&E staining and imaging of the same tissue/cells. Spatial biomarkers prognostic of tumor progression are identified. Such spatial biomarkers include pan-cytokeratin and E-cadherin. Spatial biomarkers prognostic of tumor progression are identified as indicating progressive colorectal cancer or nonprogressive or stable colorectal cancer. According to one aspect, H&E and IF same-section images are jointly analyzed to identify image features and presence of pan-cytokeratin and E-cadherin as a proxy for colorectal cancer progression. According to one aspect, anatomical annotation from H&E images (e.g., distinguishing normal tissue from a tumor) is obtained while also labeling the H&E images using visualization agent/identifiers for molecular markers. According to one aspect, many visualization agents may be used to generate high-plex data. Machine learning (ML) models generated from molecular analysis of IF images are combined with machine learning (ML) models of H&E images to aid in feature identification and interpretation, see e.g., Burlingame, E. A., Margolin, A. A., Gray, J. W. & Chang, Y. H. SHIFT: speedy histopathologicalto-immunofluorescent translation of whole slide images using conditional generative adversarial networks. Proc SPIE Int Soc Opt Eng 10581, 1058105 (2018); Prichard, J. W. et al. TissueCypher™: A systems biology approach to anatomic pathology. J Pathol Inform 6, 48 (2015). The combination of H&E images labeled with molecular markers and machine learning models identified pan-cytokeratin and E-cadherin as biomarkers that are highly predictive of a progression free survival (PFS) condition.

Further features and advantages of certain embodiments of the present invention will become more fully apparent in the following description of embodiments and drawings thereof, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1A-1E depict same-section immunofluorescence and H&E using the Orion™ Platform. FIG. 1A is a schematic of one-shot 16 to 20-channel multiplexed immunofluorescence imaging with the Orion™ method followed by Hematoxylin and Eosin (H&E) staining of the same section using an automated slide stainer and scanning of the H&E-stained slide in transillumination (brightfield) mode. This method of discriminating the emission spectra of fluorophores is repeated using seven excitation lasers spaced across the spectrum (see FIG. 7A). Using polychroic mirrors and tunable optical filters, emission spectra are extracted to discriminate up 20 channels including signal from fluorophore-labelled antibodies (15-20 in most experiments), the nuclear stain Hoechst 33342, and tissue intrinsic autofluorescence. FIG. 1B: Left panels: Orion multiplexed immunofluorescence image showing CD31, α-SMA, Hoechst (DNA), and signal from the tissue autofluorescence channel (AF) from a colorectal cancer FFPE specimen (C04); this highlights an artery outside of the tumor region with red blood cells in the vessel lumen and elastic fibers in the internal and external elastic lamina of the vessel wall, numerous smaller vessels (arterioles), and stromal collagen fibers (inset displays arterioles). Right panels: images of the H&E staining from the same tissue section (histologic landmarks are indicated). Scalebars 50 μm. FIG. 1C is an Orion multiplexed immunofluorescence image (showing CD45, pan-cytokeratin, CD31, and α-SMA) from a whole tissue FFPE section of a colorectal cancer (C04) and matched H&E from the same section. Holes in the images are regions of tissue (‘cores’) removed in the construction of TMAs. Scalebar 5 mm. FIG. 1D depicts zoom-in views of the regions indicated by arrowheads in FIG. 1C; marker combinations indicated. Scalebar 20 μm. FIG. 1E depicts intensities of fluorochromes (columns in heatmaps) in each Orion channel (rows in heatmaps) prior to (top) and after (bottom) spectral extraction. The extraction matrix was determined from control samples scanned using the same acquisition settings that were used for the full panel. The control samples included: unstained lung tissue (for the autofluorescence channel), tonsil tissue stained with Hoechst, and tonsil tissue stained in single-plex with ArgoFluor-conjugates used in the panel (for the biomarker channels). The values in each column were normalized to the maximum value in the column.

FIG. 2A-2E depict data qualifying a 16-plex single-shot Orion antibody panel. FIG. 2A depicts panels of images s from FFPE tonsil sections showing single-antibody immunohistochemistry (IHC) for pan-cytokeratin, Ki-67, CD8a, CD163, and the matching channels extracted from 16-plex Orion immunofluorescence (IF) images (H&E stain was performed on the same section as the Orion imaging). Scalebars 50 μm. FIG. 2B depicts Orion IF images and cyclic immunofluorescence (CyCIF) images from neighboring sections of an FFPE colorectal adenocarcinoma; Scalebars 50 μm. The CyCIF images were done with 2×2 binning while Orion images were obtained with no binning. FIG. 2C depicts plots of the fraction of cells positive for the indicated markers from whole slide Orion IF and CyCIF images acquired from neighboring sections from 16 FFPE colorectal cancer specimens. Pearson correlation coefficients are indicated. FIG. 2D depicts tdistributed stochastic neighbor embedding (t-SNE) plots of cells derived from CyCIF (left panels) and Orion IF images (right panels) of a FFPE colorectal cancer specimen (C01) with the fluorescence intensities of immune (CD45, pan-cytokeratin, CD8a, α-SMA) markers overlaid on the plots as heat maps. FIG. 2E depicts results of a same-slide Orion and CyCIF experiment. The tonsil samples were first processed with 16-panel Orion antibodies; PD-L1, CD4, CD8a, Ki-67, and α-SMA are shown. After imaging, fluorophores were inactivated by bleaching using the standard CyCIF protocol, then three-cycles of four-channel CyCIF staining and imaging were performed using the antibodies indicated.

FIG. 3A-3D depict data from experiments of combined H&E and Orion to identify cell/tissue types. FIG. 3A depicts representative images of Orion IF and same-section H&E from an area of normal colon (from colorectal cancer resection specimen C02). Scalebars 50 μm. FIG. 3B depicts cell types not specifically identified by markers in the Orion panel but readily recognized in H&E images including eosinophils, neutrophils, and cells undergoing mitoses (selected cells of each type denoted by arrowheads and dashed lines). Scalebars 10 μm. FIG. 3C depicts spatial maps of the positions of cells (˜15% of total cells) that were not detected by the Orion IF panel in a colorectal cancer specimen overlaid onto the corresponding H&E image (specimen: C01); red dots denote cells with identifiable nucleus but not subtyped using the antibody panel. FIG. 3D depicts in an Upper panel: Spatial map of nine tissue classes determined from the H&E image using a convolutional neural network (CNN) model for various cell types as indicated, see e.g., Kather, J. N. et al. Predicting survival from colorectal cancer histology slides using deep learning: A retrospective multicenter study. PLOS Medicine 16, e1002730 (2019). Lower panel: Percent of total of “unidentifiable” cells assigned to a specific tissue class by the CNN applied to the H&E image. FIG. 3E depicts example same-section Orion IF and H&E images from areas enriched for ‘non-detected’ cells; examples include areas predicted to be rich in stroma and smooth muscle; Scalebars 100 μm. FIG. 3F depicts Orion IF and H&E images from colorectal cancer resection specimen C26, showing an area of serrated adenoma with low pan-cytokeratin expression (markers as indicated). Whole slide image indicating the location of this region is shown in FIG. 9C. Scalebars 300 μm.

FIG. 4A-4G is directed to recapitulating and extending the Immunoscore tissue immune test using Orion images. FIG. 4A depicts a map of tumor center and invasive-margin compartments for specimen C04 overlaid on an H&E image with the density of CD3+ cells shown as a contour map (yellow) and the positions of CD8+ T cells as blue dots. The arrow indicates the zoom-in images shown below. Lower panel shows selected channels from a portion of the Orion image for C04 spanning the invasive boundary (denoted by green shading). FIG. 4B depicts a flow chart for the calculation of Image Feature Model 1 (IFM1) that recapitulates key features of the Immunoscore test. FIG. 4C depicts in a upper panel: Box-and-whisker plots for progression-free survival (PFS) for 40 CRC patients based on actual IFM1 scores (midline=median, box limits=Q1 (25th percentile)/Q3 (75th percentile), whiskers=1.5 inter-quartile range (IQR), dots=outliers (>1.5 IQR) or scores stratified into two classes as follows, low: score≤2, high: score=3 or 4 (pairwise two-tailed t-test p=0.002. Lower panel: Kaplan Meier plots computed using IFM1 binary classes (HR, hazards ratio; 95% confidence interval; logrank p-value). FIG. 4D depicts a flow chart for calculation of additional models that use the underlying logic of Immunoscore but considering 13 markers. The image processing steps are the same as in FIG. 4A. The rank positions of IFM1 and IFM2 are shown relative to all other 14,950 combinations of parameters that were considered. FIG. 4E depicts (Left) Box-and-whisker plots for PFS for 40 CRC patients based on IFM2 scores. (Right) Kaplan Meier plots computed using IFM2 binary classes stratified into two classes as follows, low: score≤2, high: score=3 or 4 (HR, hazards ratio; 95% confidence interval; logrank p-value). FIG. 4F depicts plots of leave-one-out cross-validation of ranks from IFM1 and IFM2 (unadjusted p=3.35×10-14 and adjusted using the Benjamini-Hochberg Procedure; p=5.0×10-9) and bootstrapping of hazard ratios from IFM1 and IFM2 (unadjusted p=1.94×10-25 and adjusted p=2.91×10-20). Detailed analysis was described in the methods section and pairwise two-tailed t-test were used unless otherwise mentioned. FIG. 4G depicts representative Orion IF images of cases with high IFM2 (score=4 in specimen C34) and low IFM2 (score=0 in specimen C09). IF images show DNA, pan-cytokeratin, α-SMA, CD45, and PDL1; Scalebars 100 μm.

FIG. 5A-5E are directed to a bottom-up development of a tumor-intrinsic image feature model. FIG. 5A depicts positions in specimen C39 of three selected topics identified using Latent Dirichlet Allocation (LDA). Topic locations are overlaid on an H&E image; Scalebar 5 mm. FIG. 5B depicts Left: Markers making up selected LDA topics as shown with size of the text proportional to the frequency of the marker but with colored text scaled by 50% for clarity; Radar plot indicating the fraction of cells positive for each marker in Topic 7, 8, and 11 (data for all others topics show in FIG. 11A-11B). FIG. 5C depicts immunofluorescence images showing expression of pan-cytokeratin, α-SMA, CD45, and CD20 for the indicated LDA topics. The position of each image frame is denoted by the yellow boxes in FIG. 5A. Scalebars 100 μm. FIG. 5D depicts Pearson correlation plots of progression-free survival (PFS) and Fraction of Topic 7, 8 and 11 in 40 CRC patients. Topic 11 corresponded to TLS, whose presence is known to correlate with good outcome, see e.g., Schumacher, T. N. & Thommen, D. S. Tertiary lymphoid structures in cancer. Science 375, eabf9419 (2022). FIG. 5E depicts fraction of Topics 7, 8, and 11 in colorectal cancer specimens C1-C40. FIG. 5F depicts box-and-whisker plots showing fractions of Topic 7, 8, and 11 positive cells for indicated markers; midline=median, box limits=Q1 (25th percentile)/Q3 (75th percentile), whiskers=1.5 inter-quartile range (IQR), dots=outliers (>1.5 IQR)). Pairwise t-test p values indicated.

FIG. 6A-6E are directed to data supporting LDA Topic 7 corresponding to aggressive tumor regions and is correlated with poor outcomes. FIG. 6A depicts Kaplan Meier plots of PFS for 40 CRC patients based on the fraction of Topic 7 present in the tumor domain and stratified as follows: high class: over 11% of topic 7 cells over all cells; and low class: under 11% of topic 7 cells over all cells (HR, hazards ratio; 95% confidence interval; logrank p-value). FIG. 6B depicts representative images of Topic 7 extracted from all specimens using a convolutional neural network (GoogLeNet) trained on LDA data. FIG. 6C depicts a spatial map of LDA Topic 7 and H&E image from colorectal cancer sample C02. FIG. 6D depicts a plot of fraction of Topic 7 (IFM3) versus IFM1 score for 40 CRC patients. FIG. 6E depicts Kaplan Meier plots of PFS for 40 CRC patients stratified using IFM4 which was binarized as follows: class 1: IFM1 high and Topic 7 (IFM3) low group; class 2: all other patients—i.e., either low IFM1 and/or high Topic 7 (IFM3) (HR, hazards ratio; 95% confidence interval; logrank p-value).

FIG. 7A-7E is directed to features of the fluorophores, signal extraction, antibodies, and instrumentation used in the Orion™ method. FIG. 7A is directed to emission spectra of the ArgoFluor dyes used in the experiments herein with overlaid filter profiles. Each row shows fluorophores excited using the same laser (denoted by the colored vertical line). From left to right within each laser row: 405 laser (Hoechst 33342); 445 laser (Autofluorescence); 470 laser (ArgoFluor 515, ArgoFluor 555L); 520 laser (ArgoFluor 535, ArgoFluor 550); 555 laser (ArgoFluor 572, ArgoFluor 584, ArgoFluor 602, ArgoFluor 624, ArgoFluor 660L); 640 laser (ArgoFluor 662, ArgoFluor 686, ArgoFluor 706, ArgoFluor 730); 730 laser (ArgoFluor 760, ArgoFluor 795, ArgoFluor 845, ArgoFluor 875). For the 405-laser data collection, tonsil tissue stained with Hoechst 33342 was used as the sample. For the 445-laser data collection, unstained lung tissue was used. Single color Ig-capture beads generated by incubation with antibodies conjugated to the indicated ArgoFluor dye were used as the sample for all other collections. For each sample, data was collected into multiple Orion channels spanning a wide range of wavelengths (in 2 nm center wavelength increments). FIG. 7B depicts channel images of FFPE tonsil section stained, imaged, and processed with Orion platform showing distinct spatial patterns with minimal channel crosstalk. FIG. 7C depicts stability of fluorophore and of epitope recognition in solution and in tissues for ArgoFluor 572 conjugated anti-CD4 antibody. Quantitative stability metrics were generated from three different assays to compare reagents stored at an accelerated aging condition (+21.6° C.) to reagents stored at the recommended condition (−20° C.) based on the Arrhenius equation (storage for 3.5 months at the accelerated aging condition is equivalent to 5 years at the recommended storage condition). Fluorochrome stability: The intensity of Ig-capture beads incubated with (signal) or without (background) antibody was measured from images scanned with the Orion system. The histogram overlay shows the intensity distribution for unlabeled beads (orange) and for beads incubated with antibody stored for 3.5 months at −20° C. (red) or +21.6° C. (blue). The mean fluorescence intensity (MFI) was obtained from these distributions, as well as the MFI signal-to-background (S:B) ratios. The dot plots show accelerated-to-real time CD4 MFI ratios (left plot) and S:B ratios (right plot) across 7 computed time points. Antibody binding stability: Human peripheral blood mononuclear cells (PBMC) were stained with accelerated-aged (blue) or real-time-aged (red) ArgoFluor 572 conjugated anti-CD4 antibody and analyzed using flow cytometry (3.5-month real-time/5-year accelerated time point shown in histogram. The MFI was obtained for the positive (signal) and negative (background) populations, allowing derivation of S:B ratios. The dot plots show accelerated-to-real time CD4 MFI ratios (left) and S:B ratios (right) across 7 time points. For tissue-based antibody stability testing, images of serial sections from FFPE tonsil stained with real-time aged (top) z and accelerated-aged (bottom) antibodies were obtained using the Orion system. Single cell segmentation and intensity measurements were obtained with QuPath software, and a Gaussian mixture model threshold was applied to determine positive cells (signal) from negative cells (background) to determine S:B for both conditions. These methods demonstrate equivalent performance for both storage conditions in the three assays (S:B ratio of 0.93 for the 3.5-month real-time/5-year accelerated time point).

FIG. 7D depicts a schematic of the Orion optical system. The Orion imaging system has fluorescence and brightfield imaging modes. Fluorescence imaging: The Orion system is a class 1 LASER product which uses 7-color LASER illumination (one at a time) to illuminate a sample on a microscope slide. The illumination beam emanates from the source in a fiber optic cable, then shaped with beam conditioning optics, and redirected via a beam splitter and path folding mirrors through an objective lens which focuses it onto the sample. Excitation light passing through the sample is stopped by a beam block preventing damage to the transmitted light source. Laser-excited fluorophores in the sample emit light that is collected by the objective lens. This light is redirected via the beam splitter and path folding mirrors through a tube lens for focusing, fixed and rotatable compensation elements for optical corrections, and a tunable emission filter prior to collection by a sCMOS camera. Brightfield imaging: The Orion system utilizes LED transillumination of the sample on the microscope slide. Chromogenic stains in the sample absorb a portion of the light, and the remainder is collected by the objective lens. The light follows the same path as the fluorescence emission described above, with the exception that a window is used instead of an emission filter. FIG. 7E depicts data validating minimal channel crosstalk in 18-plex tonsil image after spectral extraction. Pearson's correlation coefficients between all channel pairs were calculated using the paired pixel intensities. Square boxes with colored borders denote excitation lasers. High correlation coefficients were only found in channel pairs that contains target markers that are in close proximity.

FIG. 8A-8C depicts data Qualifying 16-plex single-shot Orion antibody panel relative to immunohistochemistry and Cyclic Immunofluorescence (CyCIF). FIG. 8A depicts panels of images from FFPE tonsil sections showing single-antibody immunohistochemistry (IHC) for the indicated markers and matching channels extracted from the 16-plex Orion immunofluorescence (IF) images (H&E stain was performed on the same section as the Orion imaging). Scalebars 50 μm. FIG. 8B depicts plots of the fraction of positive for the indicated markers (CD45, CD68, CD20, CD4) from whole slide Orion IF and CyCIF images acquired from neighboring sections from 16 FFPE colorectal cancer specimens. Pearson correlation coefficients are indicated. FIG. 8C depicts t-distributed stochastic neighbor embedding (t-SNE) plots of cells from Orion IF image (specimen: C01). Log transformed marker intensities (CD31, CD20, E-cadherin, Ki-67, pan-cytokeratin, α-SMA) were used to color the dots in each panel.

FIG. 9A-9C are directed to Orion imaging of a different disease histologies and CyCIF following Orion imaging. FIG. 9A depicts 16-plex (18 channel) Orion image from a tissue microarray (TMA) containing normal and diseased human tissues including inflammatory and neoplastic diseases (Examples highlighted are lung squamous cell carcinoma, prostate adenocarcinoma, ovarian cancer, and breast; DNA, pan-cytokeratin, KI-67, α-SMA, CD45 and CD31 are displayed. scalebars 2 mm and 400 μm, as indicated. FIG. 9B depicts Left panel: Orion image of normal colon showing E-cadherin, CD11b, CD45, CD163, Ki-67, and DNA (Sytox) signal. Right panel: same area of normal colon following inactivation of Orion fluorophores (see Methods). FIG. 9C depicts an Orion IF image from colorectal cancer resection specimen C26, showing an area of serrated adenoma with low pan-cytokeratin expression (markers as indicated). Higher magnification inset as indicated by the box is shown in FIG. 3F. Scalebar 3 mm.

FIG. 10A-10D are directed to assessment of individual markers to Image Feature Models of patient prognosis derived from Orion immunofluorescence images. FIG. 10A Upper: Ranking of 1/hazard ratio (HR) for each Image Feature Model (IFM1 to IFM14,950) calculated by determining the frequency of cells positive for one or more of 13 markers in Orion IF images lying within (tumor center: CT) or outside of a region 100 μm from the tumor invasive margin (IM) model (n=40 patients). Ranking position of IFM1 is indicated. IFM2 showed an HR=0.0785 (95% CI: 0.0358-0.172, p=1.91×10-06). Lower: Full heat map showing the selected markers at the tumor or margin in each combination. A total 14,950 combinations were generated as the set of 4 out of 26 parameters (13 markers in 2 regions). FIG. 10B depicts enrichment plots showing enrichment scores (ES) for positive cells denoted by the indicated markers (and their location in the tumor or at the tumor margin) based on the 16-plex Orion images, indicating whether the marker/location feature is enriched in the image feature models linked to the best hazard ratios. The green lines represent the running ES for a given marker/location as the analysis proceeds down the ranked list. The value at the peak is the final ES. FIG. 10C depicts a regression line scatter plot showing fraction of positive cells for indicated markers from the Orion 16-plex images vs. progression-free survival (PFS, days) for 40 patients with colorectal cancer. Each dot represents measurements from a single patient. R2 for each plot is displayed. FIG. 10D depicts representative Orion IF images of cases with high IFM2 (IS=4 in specimen C34) and low IFM2 (IS=0 in specimen C09). IF images show DNA, pan-cytokeratin, α-SMA, CD45, and PD-L1; Scalebars 200 and 300 μm as indicated. Higher magnification regions of interest are shown in FIG. 4G.

FIG. 11A-11B is directed to identifying cellular neighborhoods in colorectal cancer resections. FIG. 11A is directed to Latent Dirichlet Allocation (LDA) probabilistic modeling used to analyze Orion immunofluorescence data from 40 colorectal cancer specimens to reduce cell populations into neighborhoods (“topics”) defined by patterns of single-cell marker expression. The analysis identified 12 topics that recurred across the dataset. Within each box is the LDA plot for the indicated topic (top) and a regression line scatter plot indicating the fraction of each tumor composed of the indicated LDA topic and the relationship to progression-free survival (PFS, days). Each dot represents measurements from a single patient. R2 for each plot is displayed. FIG. 11B depicts a bar plot depicting the proportional distribution of the LDA Topics in the 40 colorectal cancer specimens.

FIG. 12A-12B is directed to evaluation of the performance of a Convolution Neural Network used to identify cellular neighborhood Topic 7 from H&E images of colorectal cancer. FIG. 12A depicts a confusion matrix table showing performance of GoogLeNet convolutional neural network (CNN) trained using H&E data from Latent Dirichlet Allocation (LDA) Topic 7 and its performance in identifying Topic 7 cells from H&E data. Topic 0 contains the rest of the topics (3, 5, 6, 9, 10, 11, 12). Target class (ground truth) was assigned from LDA analysis of Orion images and Output class (predicted) was assigned by the GoogLeNet CNN. FIG. 12B is a gallery of representative H&E images of true positives for topic 8; Scalebars 50 μm.

FIG. 13 depicts additional biomarkers of Topic 7 from CyCIF images. The sample C06 was selected based on the high fraction of Topic 7 cells from the Orion data. The CyCIF images obtained from the same specimen, but a different section are displayed.

FIG. 14 depicts the extended data in table 2. The results of the Orion™ Antibody and ArgoFluor™ Panel for Colorectal Cancer Analysis in Example II are summarized.

FIG. 15 depicts the extended data in table 3. The Colorectal Cancer Patient Characteristics in Example XVI are summarized.

FIG. 16 depicts the extended data in table 4. The CyCIF antibodies used for imaging sections 45-47 in Example XVI are summarized.

### DETAILED DESCRIPTION

According to one aspect, the disclosure provides methods for multimodal tissue imaging that combines high-plex, subcellular resolution immunofluorescence (IF) with conventional H&E imaging of the same cells. The methods may utilize many targeting antibodies in a high-plex method. According to one aspect, the method includes immunofluorescence data acquisition from a colorectal tissue sample, such as a whole slide colorectal tissue sample, while preserving the sample for high-quality same-section H&E imaging. According to one aspect, multimodal tissue imaging methods described herein leverage the use of extensive historical knowledge about tissue microanatomy based on histopathological analysis of H&E images in the interpretation of molecular data derived from molecular imaging. According to one aspect, H&E images are potentially more reliable than molecular images for the identification of some types of tumor cells. Conversely, many immune cell types cannot be reliably differentiated using H&E images, and their presence can also be difficult to discern when cells are crowded. Accordingly, the use of IF lineage markers provide critical information in these cases. According to one aspect, the complementary strengths of H&E and IF imaging can be exploited by ML/AI algorithms that are increasingly used to process tissue images in clinical and research settings, see e.g., Granter, S. R., Beck, A. H. & Papke, D. J. AlphaGo, Deep Learning, and the Future of the Human Microscopist. Arch. Pathol. Lab. Med. 141, 619-621 (2017). The present disclosure provides the use of automated image processing on H&E and molecular data to identify image features prognostic of tumor progression, see e.g., Savadjiev, P. et al. Image-based biomarkers for solid tumor quantification. Eur Radiol 29, 5431-5440 (2019), and to identify pan-cytokeratin and E-cadherin as prognostic markers for colorectal cancer.

According to the present disclosure, H&E images are used to classify cell types and states that are not readily identifiable in multiplexed data. According to the present disclosure, H&E and autofluorescence imaging are used to characterize acellular structures that organize tissues at mesoscales (e.g., the elastic lamina of the vessel wall). Conversely, by overlaying molecular data on H&E images, the methods described herein discriminate cell types that have similar morphologies but different functions. According to the present disclosure, molecular data obtained by immunofluorescence is used to label cell types in H&E images and machine learning models are used to analyze such data to indicate whether a particular colorectal cancer is progressive or is nonprogressive or stable.

For example, as described herein, Immunoscore is a pathology driven (top-down) clinical test that uses H&E and IHC data on the distribution of specific immune cell types at the tumor margin to predict outcome for patients with colorectal cancer. Immunoscore was applied to a cohort of 40 colorectal cancer individuals using automated scripts and additional immune markers to determine Hazard Ratios computed from progression free survival PFS data, see e.g., Bruni, D., Angell, H. K. & Galon, J. The immune contexture and Immunoscore in cancer prognosis and therapeutic efficacy. Nat Rev Cancer 20, 662-680 (2020). In a distinct but complementary bottom-up approach, a spatially sensitive statistical model (LDA) of IF data was used to identify cell neighborhoods significantly associated with colorectal cancer progression, and to identify pan-cytokeratin and E-cadherin, two epithelial cell markers, as markers in a tumor intrinsic model indicating progressive colorectal cancer versus nonprogressive or stable colorectal cancer. According to one aspect, the present disclosure combines the tumor-intrinsic model and the immune markers model in Immunoscore to improved the hazard ratio relative to either model used alone.

Particular methods involved in the identification of markers as described herein involve the following. Specimens (either formaldehyde fixed paraffin embedded—FFPE) or frozen section (“optimal cutting temperature—“OCT”) are sectioned 5-20 μm thick and mounted on slides. Slides are then subjected to a sodium hypochlorite bleaching step and bright white light to reduce endogenous fluorescence (autofluorescence) from specimens. Other methods of photobleaching known to those of skill in the art are contemplated. Slides are then incubated with a mixture of antibodies, each of which detects a specific antigen. According to one aspect, primary antibodies are directly conjugated to chemical fluorophores. In other aspects, the primary antibodies are detected using fluorophore-conjugated secondary antibodies.

Whole slide imaging (WSI) is performed with acquisition channel parameters (time, excitation laser, emission center wavelength (CWL), and exposure times) such that each antibody channel can be separated from the other channels and resolution is sufficient to discriminate single cells. A dye or antibody is included to label nuclei. Endogenous fluorescence is also recorded (in some cases this is done prior to the initial bleaching step). In some embodiments, spectral unmixing methods are used to separate fluorescence channels (e.g. using the Orion method). In other cases, conventional filter-based channel separation is used. In some embodiments, WSI involves scanning in the nuclear channel at low resolution to identify tissue boundaries, followed by surface mapping to identify the precise location of the tissue in x,y,z directions. In other embodiments, literally the entire area of the slide is collected. Regardless, data collection involves serially collecting fields of view (defined by the optics of the instrument and the camera dimension) and the fields of view are then stitched together and registered to create an image mosaic that covers the entirety of the tissue specimen. This ranges from 10² to 10⁶ cells.

In some embodiments, Whole tissue images are acquired at 20× following the surface map within the specified tissue boundaries by collecting all channels for a single field of view (FOV) before proceeding to the next partially overlapping FOV. Raw image files are processed to correct for system aberrations, and signals from individual targets are then isolated using the Spectral Matrix obtained with control samples, followed by stitching of FOVs to generate a continuous open microscopy environment (OME) pyramid TIFF image.

After imaging is completed, slides are de-coverslipped by immersion in 1×PBS at 37° C. until the coverslips fall away from the slide. Slides are then stained with colorimetric stains; this is performed either manually or using an auto-stainer. Hematoxylin and Eosin is the most common stain, but the present disclosure contemplates other stains/dyes known in the art.

Slides are scanned in brightfield mode, using the same scan area used for IF image acquisition. H&E images are registered to the IF images. In some embodiments, this is performed using ASHLAR and PALOM v2022.3 (world wide website github.com/labsyspharm/palom) software.

Stitching, channel registration, illumination and geometric distortion correction is performed followed by segmentation and single-cell data analysis to computed intensity of each channel on a per-cell basis as well as morphological features. In some embodiments, this is performed using MCMICRO modules with cell masks.

H&E images are then analyzed either manually by a person, to identify critical tissue features (in colorectal cancer this includes normal mucosa, invasive tumor, tumor margin, and other relevant histology features) and/or using an ML/AI algorithm that identifies and segments the normal and tumor tissue compartments. Single cell values for tumor makers (e.g. pan-cytokeratin pCK) are determined and then gated using a Gaussian Mixture Model (GMM) to identify high and low cells; assignment is confirmed by inspection of exemplary data. After gating, pCK staining is used to generate a tumor mask via a K-Nearest Neighbor (KNN) model (kernel size=25 cells). This yields the positions of spatially contiguous tumor regions. Data from analysis of H&E images can also be used at this point.

Image feature models (IFMs) are then computed using these data in different combinations. The models are scored against a measure of disease outcome such as tumor free progression (PFS). Outcome can be obtained in the absence or presence of drug therapy (with surgery setting time zero). Statistically significant associations between an image feature model (IFM) and outcome are determined using methods that account for the possibility of multi-hypothesis tests. Significant associations, as measured using Hazard Ratio in the case of PFS, represent potentially useful prognostic and predictive biomarkers.

The present disclosure contemplates computing an image feature model class I (IFM 1). IFM I is intended the capture the degree of tumor invasion/exclusion by immune cells using marker proteins previously validated by the Immunoscore assay as well as additional marker proteins reflective of current understanding of tumor biology. These marker proteins generally correspond to proteins expressed on specific immune lineages (e.g. CD4 or CD8) or to proteins reflective of the activation state of these cells (e.g. PD1, PDL1).

The tumor invasive margins (IMs) are derived from tumor masks by expanding in either direction from edge of the mask (the point of stroma-tumor contact). This defines the tumor region (TR) or the tumor boundary (TB). Regions defined by a distance of 100 μm from the boundary are used as an exemplary distance, but the distance is not critical.

The levels of the immune markers are also determined in single cells and GMM is used to binarize the data. Markers aim to compute the specific types of cell present and include (but are not limited to) CD3 for all T cells, CD8 for conventional T cells, CD4 for helper cells etc. These values are recorded for marker1, marker2 etc.

The marker1⁺ and marker2⁺ cell positive fraction is then computed for different regions of the tumor as the number of positive cells divided by the total of all successfully segmented cells of all types. In some embodiments, the median values for all samples are used to define a subscore as follows: specimens in which the ratio marker1⁺ cells to cells in the given region is below the median are scored as 0. Those above the median value are scored as 1. The process is repeated for marker2⁺ cells. In other embodiments, the threshold value for a 1 or 0 assignment is pre-defined. The final IFM I value is calculated as the sum of all subscores for marker1 and marker2 positive cells in the TR and TB regions. For two markers (the simplest effective instantiation) IFM I score therefore ranges from 0 (marker1⁺ and marker2⁺ are below median in both regions) to 4 (marker1+ and marker2⁺ are above median in both regions).

The present disclosure contemplates computing IFMs in combinatorial form (yielding IFM2). The collections of IFM class I are a more sophisticated measure of tumor invasion by immune cells that uses a wider range of marker proteins. As described herein, the markers are CD3, CD4, CD8, CD20, CD45, CD45RO, CD68, CD163, FOXP3, PD1, PD-L1, CD31, alpha-SMA, pan-cytokeratin (pCK). The levels of all measured markers (13 immune markers and 1 tumor marker) were determined, GMM was used to binarize the data and positivity for each marker was determined as for IFM1 at both the tumor region TR and the tumor boundary TB. Sets of four parameters were randomly selected and IFMs scored from 0-4 for each specimen—these were then ranked by hazard ratio. The top IFM with respect to predicting hazard ratio is then selected. The p value for the test is determined with and without correction for multi-hypothesis testing.

The top ranked IFM in this procedure generates a large number of possible marker combinations, of which a subset (526 in the present disclosure) outperform IFM1 with respect to Hazard Ratio (˜0.15). These are potentially all useful biomarkers combinations and the final value is selected based on the signal to noise ratio of individual measurements that make up the combination. This is hypothesized to result in the most robust prognostic combination biomarker.

With IFM I combinations, it was observed that immune cells in both the TR and TB regions contributed to the final score (1035 & 1069 counts, respectively). With respect to combinations biomarkers as a whole, CD3, CD8 and SMA are the highest contributors from tumor region TR, as CD45, PD-L1 and CD163 are the highest contributors from tumor boundary TB. This is interpreted as measuring the density of T conventional cells inside the tumor region and properties of T cells and macrophages in the region surrounding the tumor.

A test cohort with known clinical outcome (e.g. PFS) is used to select specific embodiments of IFM2 and this is then confirmed on a second independent validation cohort.

The present disclosure contemplates computing IFM Class II (e.g. IFM3). IFMs in the second class aim to capture information about tumor cells themselves as well as their immediate local environments. This contrasts with the Class I IFMs (above) that focus on immune cells. IFM II models are identified by a machine learning approach as being significantly associated with disease progression. They comprise a set of the ratios of multiple marker proteins scored on a continuous scale. The highest performing IFM (IFM3) is characterized by a high probability that pCK^{high} and E-cadherin^{low} cells will be found in proximity to each other. In one embodiment, the non-uniform ratio of pCK^{high} and E-cadherin^{low} is determined and defined with respect to normal tissue, which can often be found adjacent to the tumor itself or can be measured in a parallel normal control. In IFM3 high cells, both low cadherin levels relative to pCK levels in single cells and the proximity of cells with very different ratios of these proteins were observed. In normal tissue, this ratio is quite stable. IFM3 identified tumor cells that, upon examination by a pathologist, had a very distinctive “discohesive” phenotype in which tumor cells have lost their normal contacts and arrangements with respect to each other. Importantly, this can be rationalized as a property of cells with low cadherin levels making the machine-learned IFM3 model “interpretable.” Interpretability/explainability is increasingly thought to be a key feature of any medical use of machine learning AI. Of note, it is likely that tumor markers other than pCK and cohesion proteins other than E-cadherin can be used in the same manner to create other similar biomarkers.

Potential predictive combinatorial biomarkers in the IFM II set were derived from a machine learning approach (Latent Dirichlet Allocation; spatial LDA) in which single-cell data are partitioned into grids (200 μm×200 μm in the current study) and then analyzed for the spatial distribution of individual biomarker proteins (CD3, CD4, CD8. CD20 CD45, CD45RO, CD68, CD163, FOXP3, PD1, PD-L1, CD31, alpha-SMA, pCK, E-cadherin, and Ki67) to recover the recurrent arrangements that are most predictive of tumor progression. This approach identifies both immune and tumor features while tumor features are most likely to be complementary to Class I IFMs.

Contiguous domains of cells (within 200×200 μm²) in which high probability of CK⁺ and E-cadherin⁺ cells are presented while the ratio of CK⁺/E-cadherin⁺ levels>1 are then identified. The number of these cells in these domains relative to the total number of cells in the specimen is then determined. This is repeated for all specimens in the cohort.

In some embodiments, the median value of the relevant LDA topic is determined for all specimens in the cohort and those above the median value are IFM3-high; those below are IFM3-low. IFM3-low tumors were observed to have an HR ratio of 0.26 relative to IFM3 high tumors. The H&E images of regions high in Topic 7 are examined by human experts to identify the associated morphology and provide an explanatory element. For Topic 7 this is observed to be a discohesive tumor morphology.

The present disclosure contemplates computing IFM Class III. IFM III was intended as a composite score that included both tumor-intrinsic and immune features of tumors. IFM Class I and IFM Class II were uncorrelated, and thus, a composite score is developed compared to either score alone.

IFM 4 is computed from IFM1 and IFM3 using a simple classification scheme. Class 1: IFM3 low and IFM1>1; Class 2: all other tumors.

The present disclosure is directed to a method including the steps of determining presence of pan-cytokeratin and E-cadherin in tumor cells and nontumor cells of a colorectal tissue sample obtained from a human patient by (i) contacting pan-cytokeratin with an antibody to pan-cytokeratin and detecting binding between pan-cytokeratin and the antibody to pan-cytokeratin and (ii) contacting E-cadherin with an antibody to E-cadherin and detecting binding between E-cadherin and the antibody to E-cadherin, determining levels of pan-cytokeratin and E-cadherin in the nontumor cells of the colorectal tissue sample and determining a ratio of pan-cytokeratin and E-cadherin in the nontumor cells of the colorectal tissue sample to establish a nontumor cell control baseline ratio, determining levels of pan-cytokeratin and E-cadherin in the tumor cells of the colorectal tissue sample and determining a ratio of pan-cytokeratin and E-cadherin in the tumor cells of the colorectal tissue sample to establish a tumor cell ratio, and comparing the tumor cell ratio to the nontumor cell control baseline ratio.

According to one aspect, a tumor cell ratio that is greater than the nontumor cell control baseline ratio indicates that the tumor cells are progressive colorectal cancer tumor cells.

According to one aspect, a tumor cell ratio that is substantially similar to the nontumor cell control baseline ratio indicates that the tumor cells are nonprogressive or stable colorectal cancer tumor cells. “Substantially similar” as used herein is understood as is known in the art to mean that the tumor cell ratio not statistically different from the nontumor cell ratio by a standard test at a pre-defined level of confidence. The standard test is a Students t-test with p=0.05. With this p value, there is a 5% chance of an erroneous conclusion. According to an exemplary aspect, a tumor cell ratio that is not statistically different from the nontumor cell ratio using a Students t-test with p=0.05 indicates that the tumor cells are nonprogressive or stable colorectal cancer tumor cells.

According to one aspect, the colorectal tissue sample is a whole slide colorectal tissue sample.

According to one aspect, the colorectal tissue sample is stained to identify tissue morphology after being contacted with the antibodies.

According to one aspect, a tumor cell ratio that is greater than the nontumor cell control baseline ratio indicates that the tumor cells are progressive colorectal cancer tumor cells, and the patient is treated for colorectal cancer with surgery.

According to one aspect, a tumor cell ratio that is greater than the nontumor cell control baseline ratio indicates that the tumor cells are progressive colorectal cancer tumor cells, and the patient is treated for colorectal cancer with chemotherapy.

According to one aspect, a tumor cell ratio that is greater than the nontumor cell control baseline ratio indicates that the tumor cells are progressive colorectal cancer tumor cells, and the patient is treated for colorectal cancer with radiation therapy.

According to one aspect, a tumor cell ratio that is greater than the nontumor cell control baseline ratio indicates that the tumor cells are progressive colorectal cancer tumor cells, and the patient is treated for colorectal cancer with immunotherapy.

According to one aspect, a tumor cell ratio that is greater than the nontumor cell control baseline ratio indicates that the tumor cells are progressive colorectal cancer tumor cells, and the patient is treated for colorectal cancer with adjuvant therapy.

According to one aspect, a tumor cell ratio that is greater than the nontumor cell control baseline ratio indicates that the tumor cells are progressive colorectal cancer tumor cells, and the patient is treated for colorectal cancer with neoadjuvant therapy.

According to one aspect, a low value for E-cadherin in the tumor cells compared to the nontumor cells indicates that the tumor cells are progressive colorectal cancer tumor cells.

According to one aspect, a ratio of pan-cytokeratin to E-cadherin in tumor cells is determined normalized to a ratio of pan-cytokeratin to E-cadherin in nontumor cells, and the ratio of pan-cytokeratin to E-cadherin in tumor cells is greater than 1.

According to one aspect, a ratio of E-cadherin to pan-cytokeratin in tumor cells is determined normalized to a ratio of E-cadherin to pan-cytokeratin in nontumor cells, and the ratio of E-cadherin to pan-cytokeratin in tumor cells is less than 1.

The present disclosure provides a method of distinguishing progressive cancer cells from nonprogressive cancer cells in a colorectal tissue sample from a human patient including the steps of determining presence of pan-cytokeratin and E-cadherin in tumor cells and nontumor cells of the colorectal tissue sample by (i) contacting pan-cytokeratin with an antibody to pan-cytokeratin and detecting binding between pan-cytokeratin and the antibody to pan-cytokeratin and (ii) contacting E-cadherin with an antibody to E-cadherin and detecting binding between E-cadherin and the antibody to E-cadherin, determining levels of pan-cytokeratin and E-cadherin in the nontumor cells of the colorectal tissue sample and determining a ratio of pan-cytokeratin and E-cadherin in the nontumor cells of the colorectal tissue sample to establish a nontumor cell control baseline ratio, determining levels of pan-cytokeratin and E-cadherin in the tumor cells of the colorectal tissue sample and determining a ratio of pan-cytokeratin and E-cadherin in the tumor cells of the colorectal tissue sample to establish a tumor cell ratio, and comparing the tumor cell ratio to the nontumor cell control baseline ratio, wherein a tumor cell ratio that is greater than the nontumor cell control baseline ratio indicates that the tumor cells are progressive colorectal cancer tumor cells, and wherein a tumor cell ratio that is substantially similar to the nontumor cell control baseline ratio indicates that the tumor cells are nonprogressive colorectal cancer tumor cells.

According to one aspect, the method further includes the step of treating the patient for colorectal cancer with one or more of surgery, chemotherapy, radiation therapy, immunotherapy, adjuvant therapy or neoadjuvant therapy.

The present disclosure provides a method of detecting a pattern of biomarkers within a colorectal tissue sample of a human, wherein the colorectal tissue sample comprises tumor cells and nontumor cells, wherein the biomarkers comprise pan-cytokeratin and E-cadherin, including the steps of contacting the colorectal tissue sample with an antibody that identifies presence in the colorectal tissue sample of pan-cytokeratin, contacting the colorectal tissue sample with an antibody that identifies presence in the colorectal tissue sample of E-cadherin, acquiring pattern detection of the antibody that identifies presence in the colorectal tissue sample of pan-cytokeratin, and acquiring pattern detection of the antibody that identifies presence in the colorectal tissue sample of E-cadherin.

The present disclosure provides a method of assaying for pan-cytokeratin and E-cadherin within a colorectal tissue sample of a human, wherein the colorectal tissue sample comprises tumor cells and nontumor cells, including the steps of contacting the colorectal tissue sample with a first antibody that identifies presence in the colorectal tissue sample of pan-cytokeratin, contacting the colorectal tissue sample with a second antibody that identifies presence in the colorectal tissue sample of E-cadherin, determining amount of pan-cytokeratin relative to amount of E-cadherin within the tumor cells of the colorectal tissue sample, and determining amount of pan-cytokeratin relative to amount of E-cadherin within the nontumor cells of the colorectal tissue sample. According to one aspect, the method further includes the step of comparing (i) the amount of pan-cytokeratin relative to the amount of E-cadherin within the tumor cells of the colorectal tissue sample to (ii) the amount of pan-cytokeratin relative to the amount of E-cadherin within the nontumor cells of the colorectal tissue sample. According to one aspect, the colorectal tissue sample is a whole slide colorectal tissue sample. According to one aspect, the colorectal tissue sample is stained to identify tissue morphology after being contacted with the antibodies.

The present disclosure provides a method of treating a human for progressive colorectal cancer including the steps of assaying for a ratio of pan-cytokeratin to E-cadherin within tumor cells of a colorectal tissue sample, assaying for a ratio of pan-cytokeratin to E-cadherin within nontumor cells of the colorectal tissue sample, and treating the human for progressive colorectal cancer when the ratio of pan-cytokeratin to E-cadherin within tumor cells of the colorectal tissue sample is higher than the ratio of pan-cytokeratin to E-cadherin within nontumor cells of the colorectal tissue sample.

The present disclosure provides a method of treating a human for progressive colorectal cancer including the steps of assaying for a ratio of pan-cytokeratin and E-cadherin within tumor cells of a colorectal tissue sample, assaying for a ratio of pan-cytokeratin and E-cadherin within nontumor cells of the colorectal tissue sample, and treating the human for progressive colorectal cancer when the ratio of pan-cytokeratin and E-cadherin within tumor cells of the colorectal tissue sample is different than the ratio of pan-cytokeratin and E-cadherin within nontumor cells of the colorectal tissue sample.

The present disclosure provides a combination of a labeled antibody to pan-cytokeratin and a labeled antibody to E-cadherin for the staining of a colorectal tissue sample.

The present disclosure provides a colorectal tissue sample from a human suspected of having colorectal cancer that is stained with a labeled antibody to pan-cytokeratin and a labeled antibody to E-cadherin.

The present disclosure provides a kit for use in an immunofluorescence assay including at least a first labeled antibody to pan-cytokeratin, a second labeled antibody to E-cadherin, and optional instructions for contacting the first labeled antibody to pan-cytokeratin and the second labeled antibody to E-cadherin to a tissue sample. According to one aspect, the first labeled antibody to pan-cytokeratin is in a suitable liquid medium, and the second labeled antibody to E-cadherin is in a suitable liquid medium. According to one aspect, the first labeled antibody is in a first vessel and the second labeled antibody is in a second vessel. According to one aspect, the first labeled antibody and the second labeled antibody are combined in a vessel.

The following examples illustrate exemplary aspects of the present disclosure without, however, limiting the scope thereof.

### EXAMPLE I

### Tissue Preparation

Blocks of FFPE tonsil (AMSBIO, cat #6022CS) and lung adenocarcinoma (AMSBIO, cat #28004) and colorectal adenocarcinoma from the BWH Pathology Department archives were cut at 5 μm thickness using a rotary microtome and the sections were mounted onto Superfrost™ Plus microscope glass slides (Thermo Fisher, Catalog No. 12-550-15). The slides were dried at 37° C. overnight and baked at 59° C. for one hour. Slides were stored at 4° C. until use.

### EXAMPLE II

### Fluorophores for Orion™ Imaging

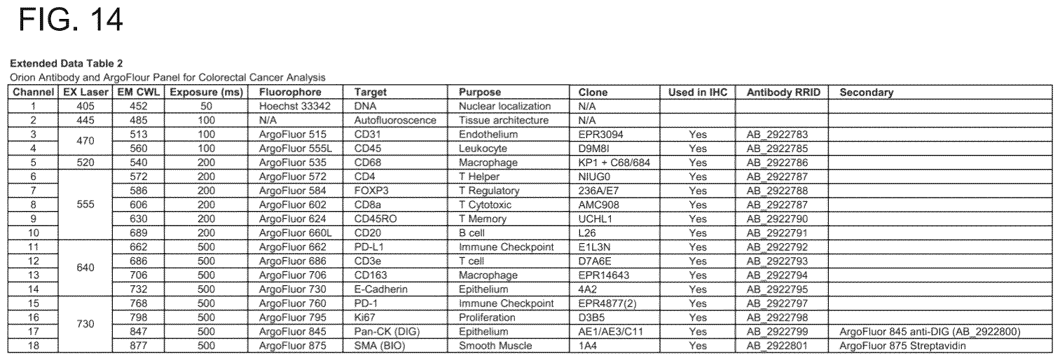

The Orion™ instrument is designed to work with an optimized set of fluorophores from RareCyte, branded as ArgoFluor™ dyes whose emission peaks cover the spectrum from green to far-red (Extended Data Table 2; FIG. 14). Although the instrument can also be used with other commercially available dyes, the ArgoFluor™ dyes have been strategically chosen based on a combination of properties that include resistance to photobleaching, narrow excitation and emission spectra, and high quantum efficiency. See e.g., Schürch, C. M. et al. Coordinated Cellular Neighborhoods Orchestrate Antitumoral Immunity at the Colorectal Cancer Invasive Front. Cell 182, 1341-1359.e19 (2020). ArgoFluor™ dyes are optimized.

### EXAMPLE III

### Immunofluorescence Antibodies

Antibodies were obtained in carrier-free PBS and conjugated directly to either biotin for a-SMA, digoxygenin for pan-cytokeratin or to ArgoFluor™ dyes (RareCyte, Inc.) using amine conjugation chemistry. After determining labeling efficiency using absorbance spectroscopy, the conjugated antibodies were diluted in PBS-Antibody Stabilizer (CANDOR Bioscience GmbH, Catalog No. 130050) to a concentration of 200 μg/mL. Antibodies used in immunofluorescence studies are listed in the Extended Data Table 2, FIG. 14.

### EXAMPLE IV

### Immunofluorescence Staining

Slides were de-paraffinized and subjected to antigen retrieval for 5 minutes at 95° C. followed by 5 minutes at 107° C., using pH8.5 EZ-AR 2 Elegance buffer (BioGenex, Catalog No. HK547-XAK). To reduce tissue autofluorescence, slides were placed in a transparent reservoir containing 4.5% H2O2 and 24 mM NaOH in PBS and illuminated with white light for 60 minutes followed by 365 nm light for 30 minutes at room temperature as previously described, see e.g., Lin, J. R. et al. Highly multiplexed immunofluorescence imaging of human tissues and tumors using t-CyCIF and conventional optical microscopes. Elife 7, e31657 (2018). Slides were rinsed with surfactant wash buffer (0.025% Triton X-100 in PBS), placed in a humidified stain tray, and incubated in Image-iT™ FX Signal Enhancer (Thermo Fisher, Catalog No. 136933) for 15 minutes at room temperature. After rinsing with surfactant wash buffer, the slides were placed in a humidity tray and stained with the panel of fluor and hapten-labeled primary antibodies in PBS-Antibody Stabilizer (CANDOR Bioscience GmbH, Catalog No. 130 050) containing 5% mouse serum and 5% rabbit serum for 2 hours at room temperature. Slides were then rinsed again with surfactant wash buffer and placed in a humidified stain tray and Incubated with Hoechst 33342 (Thermo Fisher Catalog no. H3570), ArgoFluor™ 845 mouse-anti-DIG, and ArgoFluor™ 875-conjugated streptavidin in PBS-Antibody Stabilizer containing 10% goat serum for 30 minutes at room temperature. The slides were then rinsed a final time with surfactant wash buffer and PBS, coverslipped with ArgoFluor™ Mounting Media (RareCyte, Inc.) and dried overnight.

### EXAMPLE V

### ArgoFluor™-Antibody Conjugate Stability Testing

Antibody accelerated-aging studies were performed to determine ArgoFluor™-antibody conjugation stability. Reagent stability was measured using the ratio of quantitative metrics obtained with the accelerated condition (21.6° C.) to those obtained with the storage condition (−20° C.). Tissue validation (Orion IF): Single-cell mean fluorescence intensity (MFI) data obtained by imaging FFPE tonsil stained with the ArgoFluor™ conjugate was gated using a Gaussian mixture model to obtain the percent of positive cells and S:B values (S and B refer to the MFI of cells with values above (S, Signal) and below (B, Background) the gated threshold). Fluor stability (Orion IF): Single bead MFI data was obtained by imaging Ig-capture beads incubated with(S) or without (B) the ArgoFluor™ conjugate. Binding stability (Flow Cytometry): Intensity data from peripheral blood mononuclear cells (PBMC) stained with the ArgoFluor conjugated antibody was manually gated to obtain % Positive and S:B values (S and B refer to the MFI of cells with values above(S) and below (B) the gated threshold).

### EXAMPLE VI

### The Orion Method and Instrumentation

The Orion instrument was designed with the following performance goals: (1) whole-slide imaging; (2) rapid single-pass data collection; (3) sub-cellular imaging resolution; (4) sufficient immunoprofiling depth; (5) bright-field imaging; (6) optical and mechanical stability for accurate image tile stitching; and (7) compatibility with established image data standards and formats. ArgoFluor™-conjugated antibodies along with Hoechst dye and tissue autofluorescence were excited by seven laser lines, ranging from 405 to 730 nm (Extended Data Table 2, FIG. 14). To separate the overlapping emission spectra, images were captured through a set of nine bandpass filters, which can achieve a tunable narrow band detection window (10-15 nm) throughout the spectrum from 425 nm to 894 nm. For a specific sample, the detection bands were chosen to optimize color separation, implemented with RareCyte Inc.'s Artemis™ software. Tuning of these filters is based on the well-known fact that the spectrum of a thin-film interference filter shifts toward shorter wavelengths when the angle of incidence shifts away from 0 degrees (orthogonal to the filter surface). The filters were motorized such that any narrow band of 10-15 nm can be achieved across the entire fluorescence spectrum. Narrow bandpass emission channels improve specificity; the resulting lower signal is overcome by using high power excitation lasers, which yield power at the sample plane ranging from 270 to 600 mW, more than 10 times greater than a typical fluorescence microscope.

### EXAMPLE VII

One-Shot Antibody IF Imaging with the Orion Instrument

Whole slides were scanned using the Orion instrument using acquisition settings optimized for the specific antibody panels. Briefly, acquisition channel parameters were defined for each biomarker plus an additional channel dedicated to tissue autofluorescence, and included excitation laser, emission center wavelength (CWL), and exposure times. The nuclear channel was scanned at low resolution to identify tissue boundaries, followed by surface mapping at 20× to find the tissue in the z-axis. Whole tissue was acquired at 20× following the surface map within the specified tissue boundaries by collecting all channels for a single field of view (FOV) before proceeding to the next partially overlapping FOV. Raw image files were processed to correct for system aberrations, then signal from individual targets were isolated to separate channels using the Spectral Matrix obtained with control samples, followed by stitching of FOVs to generate a continuous open microscopy environment (OME) pyramid TIFF image.

### EXAMPLE VIII

### Same Section H&E Staining and Imaging

After Orion imaging was complete, slides were de-coverslipped by immersion in 1×PBS at 37° C. until the coverslips fell away from the slide. Slides were rinsed in distilled water for 2 minutes, then stained by a routine regressive H&E protocol using Harris Hematoxylin (Leica, Catalog No. 3801575) and alcoholic eosin Y (Epredia, Catalog No. 71211). Coverslipping was performed with toluene-based mounting media (Thermo Scientific, Catalog No. 4112). After drying for 24 hours, slides were scanned on an Orion system in brightfield mode, using the same scan area used for IF image acquisition. H&E images were also acquired using an Aperio GT450 microscope (Leica Biosystems), and the H&E images were registered to the IF images using ASHLAR41 and PALOM software (world wide website github.com/Yu-AnChen/palom).

### EXAMPLE IX

### Pathology Annotation of H&E Images Performed After Orion Immunofluorescence Imaging

H&E images were annotated by a board-certified anatomic pathologist (SC and SS). The histologic features of each tissue section were defined and labeled in OMERO Path Viewer software on whole slide images according to morphologic criteria, see e.g., Digestive System Tumours: WHO Classification of Tumours. (World Health Organization, 2019), including normal mucosa, hyperplastic mucosa, adenomatous mucosa (tubular or serrated), invasive adenocarcinoma (tumor), lymphovascular invasion (LVI), peri-neural invasion (PNI), secondary lymphoid structures/Peyer's patches (SLS), tertiary lymphoid structures (TLS), lymphoid aggregates (without identifiable germinal center formation), lymph nodes. Tertiary lymphoid structures were morphologically defined by the presence of a lymphoid aggregate with germinal center formation and an anatomic distribution and appearance inconsistent with a secondary lymphoid structure (Peyer's patch or lymph node).

### EXAMPLE X

### Immunohistochemistry

FFPE sections were de-paraffinized, dehydrated, and endogenous peroxidase activity was blocked. Antigen retrieval was performed for 20 minutes at 100° C., at pH9, using BOND Epitope Retrieval Solution 2 (Leica Biosystems). Detection was achieved using a Bond Polymer Refine Detection® DAB chromogen kit and counterstained with hematoxylin. Slides were scanned using a RareCyte CyteFinder instrument. Primary antibodies used in immunohistochemistry are listed in Extended Data Table 2, FIG. 14.

### EXAMPLE XI

### Orion Image Processing Data Quantification

Image stitching and segmentation. Image data processing was performed using MCMICRO modules, see e.g., Schapiro, D. et al. MCMICRO: a scalable, modular image-processing pipeline for multiplexed tissue imaging. Nat Methods 19, 311-315 (2022). Briefly, stitched, registered, illumination and geometric distortion corrected images were generated by the Orion platform. Single-cell segmentation was performed with UNMICST2 and cell masks were generated by 5-pixel dilation of the nucleus masks. Mean intensity of each channel and morphological features were quantified for each cell masks. Image and data analysis was performed using customized scripts in Python, ImageJ and MATLAB. All code is available on GitHub (world wide website github.com/labsyspharm/orion-crc).

### EXAMPLE XII

### Analysis of Channel Crosstalk

Single-plex tonsil images. Tonsil FFPE sections stained with single antibody-ArgoFluor underwent standard acquisition and extraction process using the Orion instrument. The pixel intensities of all 18 channels from 17 samples were used to quantify bleed through of a given antibody-ArgoFluor complex to the other channels before and after spectral extraction. 18-plex tonsil image. Pearson's correlation coefficients between all channel pairs were computed using pixel intensities in the 18-plex tonsil image before and after spectral extraction.

### EXAMPLE XIII

### Computational Analysis of Orion Images and Derivation of Image Feature Models (IFM)

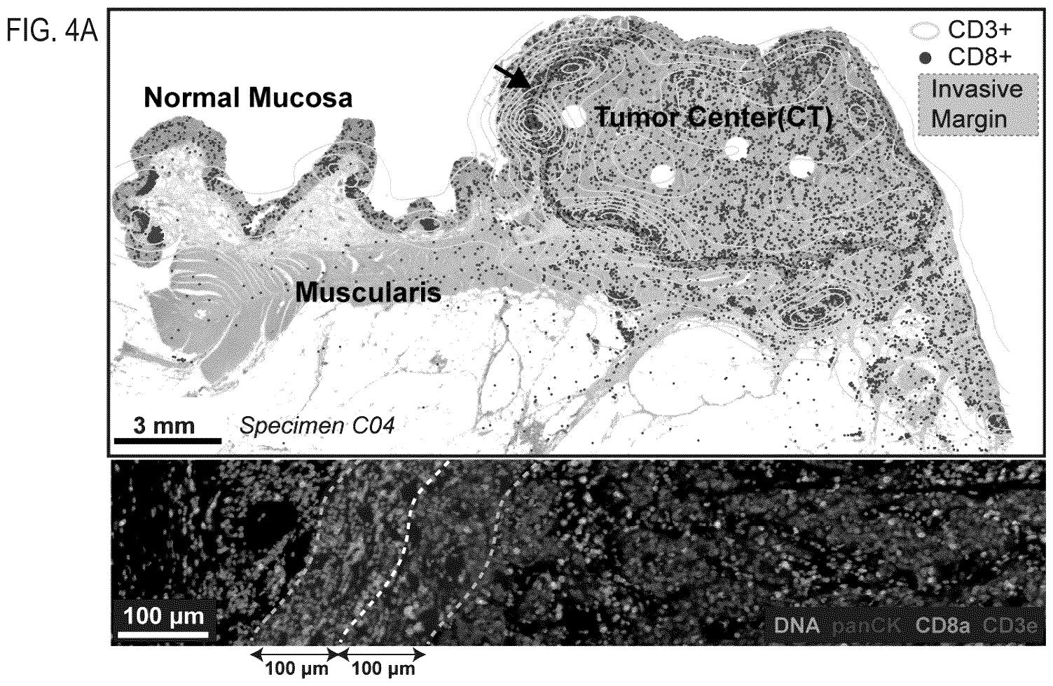

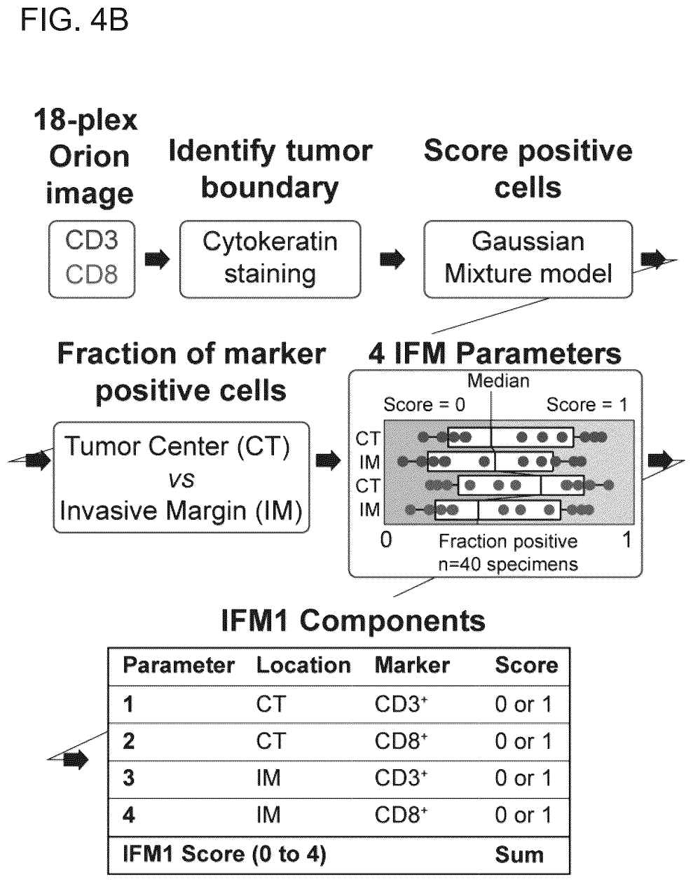

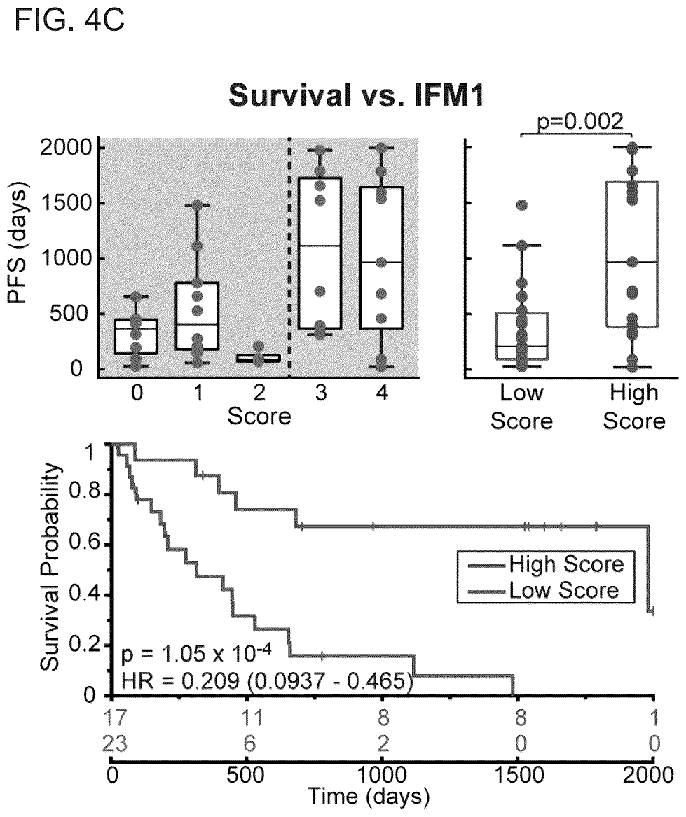

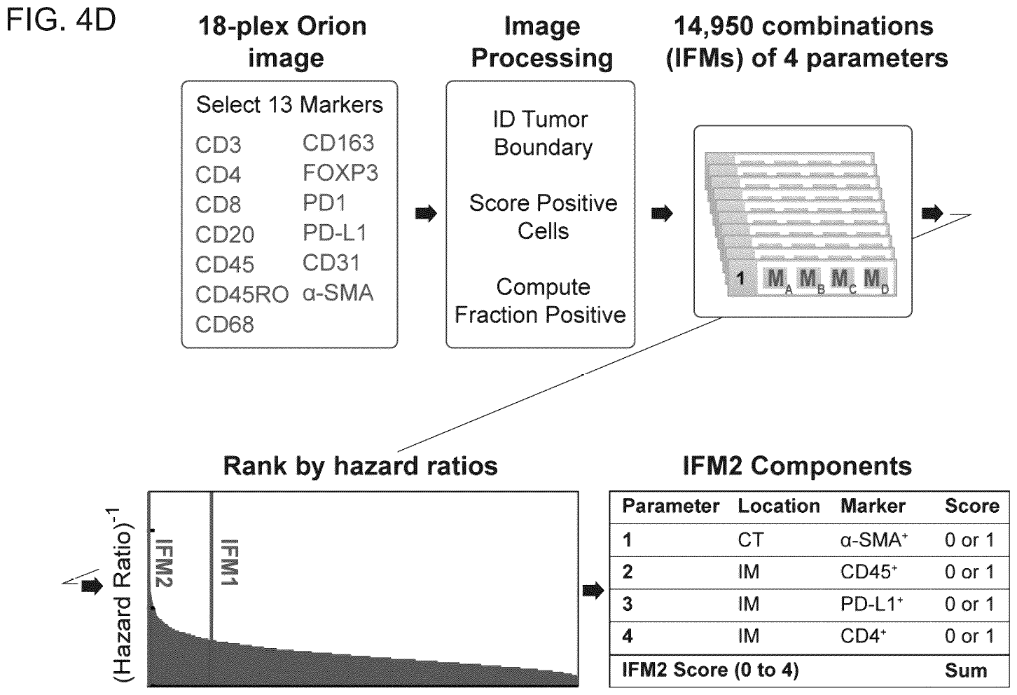

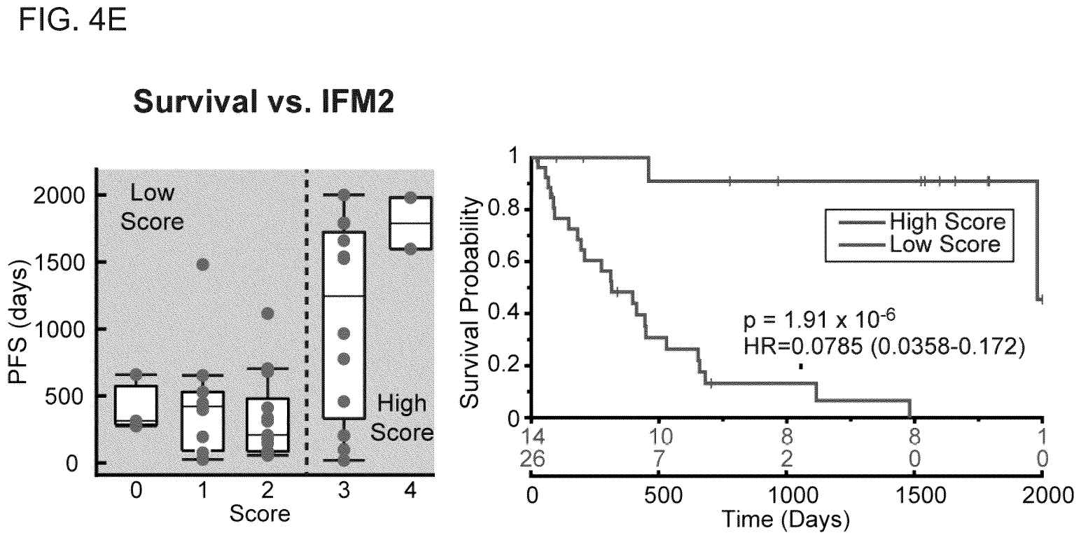

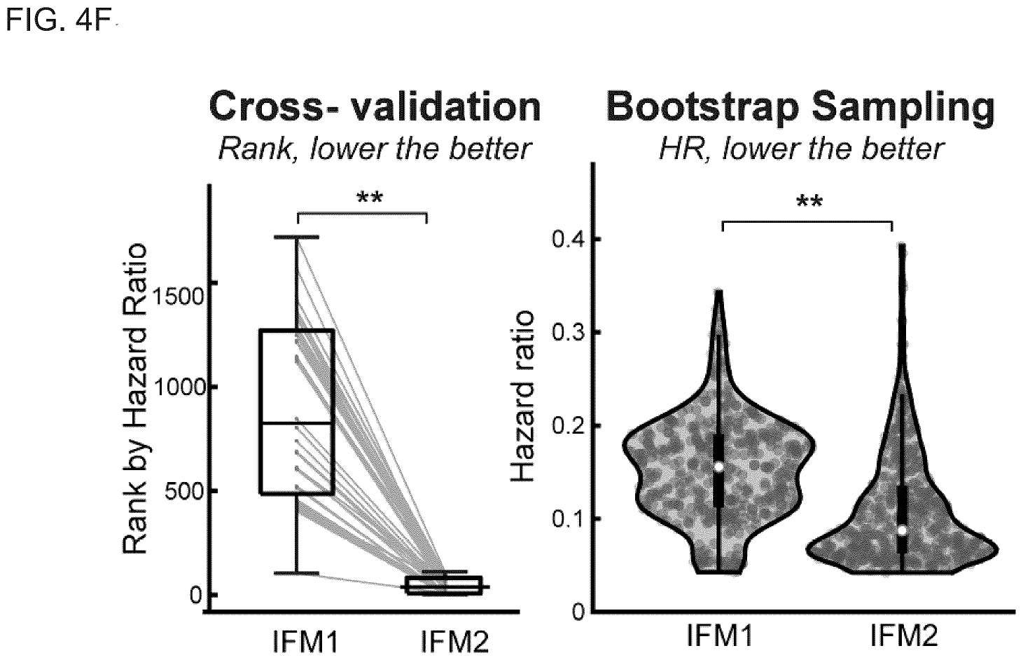

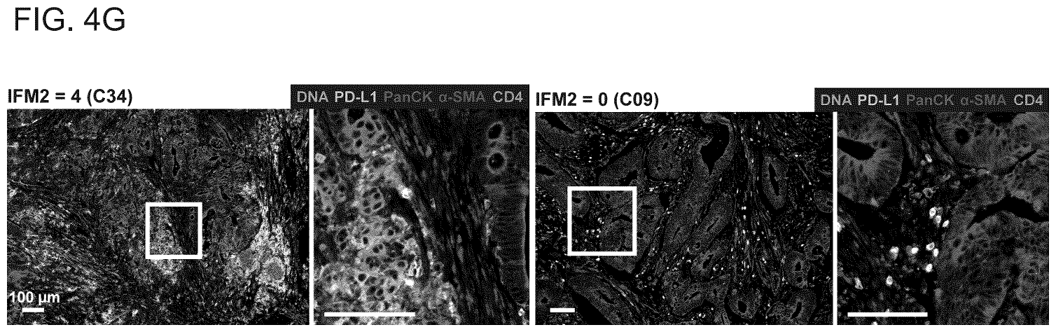

IFM computation from Orion data. IFM1 was designed to replicate the logic of the Immunoscore method and was calculated in a semi-automated manner using Orion data. In brief, quantitative data of tumor and immune markers (pan-cytokeratin, CD3e, and CD8a) were gated for high and low cells using a Gaussian Mixture Model (GMM) and confirmed by inspection. After gating, the pan-cytokeratin+ cells were then used to generate tumor masks using a K-Nearest Neighbor (KNN) model (kernel size=25 cells). The tumor margins were derived from tumor masks by expanding 100 microns in either direction from the point of stroma-tumor contact. The CD3+ and CD8+ fraction, defined as marker positive cells divided by the total of all successfully segmented cells of all types in either the tumor center (TC) or invasive margin (IM). Tumor and margins were enumerated independently in each sample. The median values of all samples were used as a cutoff to defined a subscore as follows: below the median scored as 0 and above the median scored as 1. The final IFM1 value was calculated by adding together the subscores for CD3 and CD8 positive cells in the TC and IM regions (see FIG. 4B for a flow diagram). The IFM1 score therefore ranged from 0 (CD3+ and CD8+ low in both regions) to 4 (CD3+ and CD8+ high in both regions). Similar logic was used to generated other combinations of IFMs. 13 selected immune markers (CD3, CD8, CD45, CD45RO, CD68, CD163, CD4, CD20, α-SMA, FOXP3, PD-1, PD-L1) were gated as described above, and 26 parameters (each marker in the tumor or tumor/stromal interface regions) were generated. The complete combination of 4 out of 26 parameters was tested against PFS days for Hazard Ratio (HR). IFM2 was the 3rd best IFM among those combinations, excluding the 1st and 2nd best combinations which had some of the same markers as IFM1 (i.e., CD3 and CD8); the difference in performance between the top performing models was insignificant.

Leave-one-out (LOO) test and bootstrapping analysis for IFM2. In the LOO test, the ranks of IFM1 and IFM2 were recalculated with the 40 set of samples (n=39); each set left out one sample from the original cohort. The collections of ranks from IFM1 and IFM2 were then tested with pairwise t-test. For bootstrapping, the 500 set of randomly selected samples were used to recalculate the hazard ratios of IFM1 and IFM2 as described above. The collections of hazard ratios from IFM1 and IFM2 were then tested with the pairwise t-test. To adjust for multiple hypotheses, the Benjamini-Hochberg Procedure was used with FDR=0.1

Latent Dirichlet Allocation for IFM3 and IFM4. Latent Dirichlet Allocation (LDA) was used to compute spatial neighborhoods as described, see e.g., Lin, J. R. et al. Multiplexed 3D atlas of state transitions and immune interactions in colorectal cancer. bioRxiv 2021.03.31.437984 (2021) doi: 10.1101/2021.03.31.437984. First, each sample was divided into “grids” of 200 microns², and marker frequency was calculated in each grid. The summarized probabilities of all samples were then used to generate the LDA model with 12 topics using collapsed Gibbs sampling in MATLAB. The optimal topic number was determined via varying numbers (between 8 to 16) of topics and evaluating the goodness-of-fit by calculating the perplexity of a held-out set. After fitting a global LDA model, the individual samples were then applied with the same models to assign topics at the single-cell level.

### EXAMPLE XIV

### Convolutional Neural Network to Identify IFM3 in H&E Images

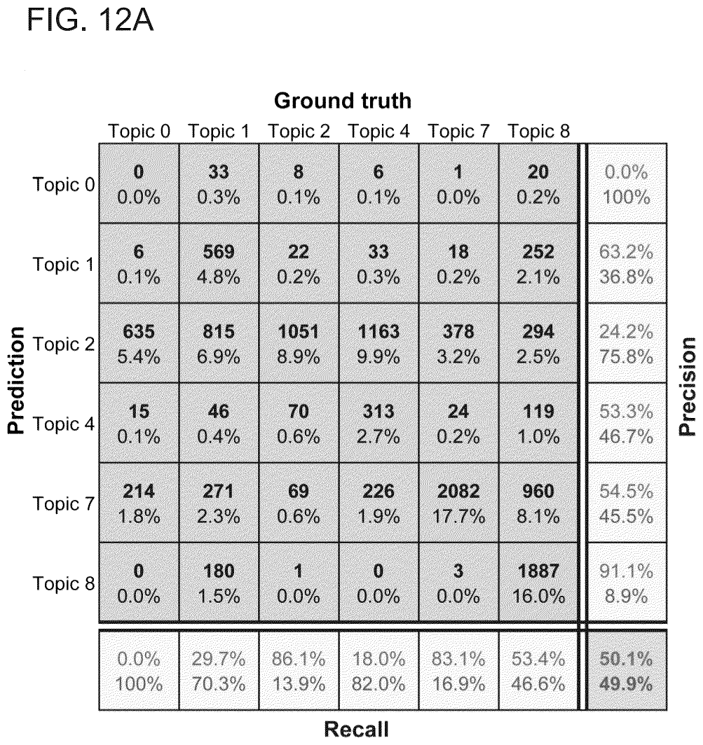

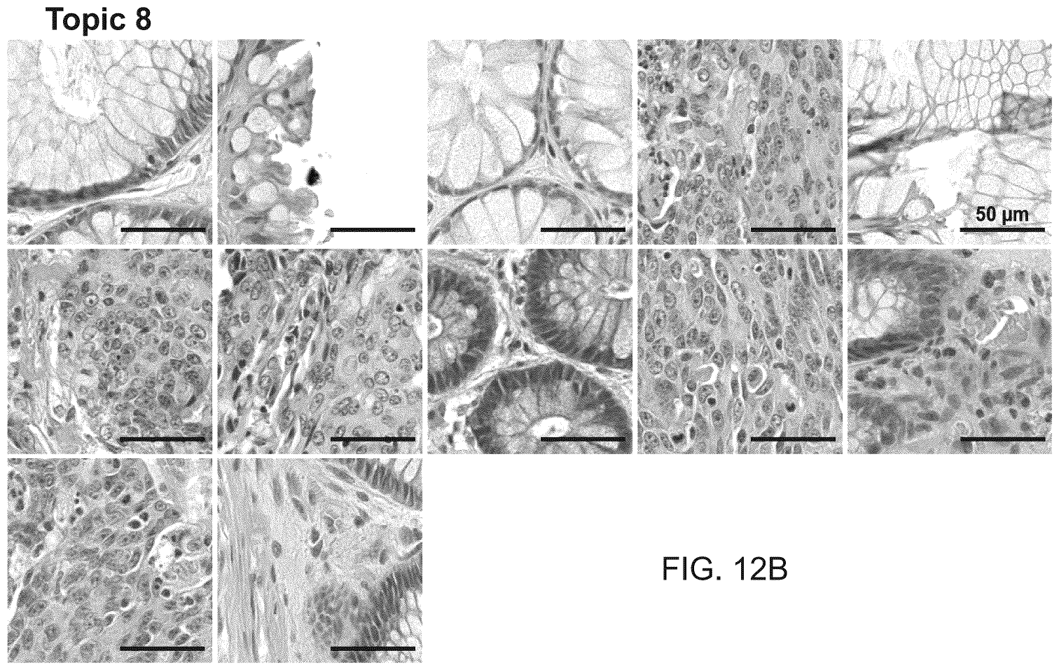

The transfer learning of a GoogLeNet model was done as follows. First, the patch images of 224×224 pixels² were generated from post-Orion H&E images, and the LDA topics were assigned to each patch using Orion data. To exclude low confidence training data, only patches with more than 20 cells and the percentage of the dominant topic over 60% were used. The selected patches were than separated into training, validation, and test sets as the ratio 0.6:0.2:0.2. The training was done with MATLAB (version 2019b) and the results are shown in FIG. 12A. Scripts and training data are available at world wide website github.com/labsyspharm/orion-crc.

A publicly available DenseNet161 model (https://doi.org/10.1101/2021.12.23.474029) trained with the 100K CRC H&E dataset (https://doi.org/10.5281/zenodo.1214456) was used to classify the post-Orion H&E image patches (112 μm²) for all the colorectal cancer samples. WSI patch prediction was performed with TIAToolbox v1.1.0 (https://doi.org/10.1101/2021.12.23.474029) on a Windows PC with Nvidia Geforce GTX 1080 graphics card and using batch size=32. Model performance was reported as F1=0.992. As described in the training dataset, there are 9 output classes: adipose (ADI), background (BACK), debris (DEB), lymphocytes (LYM), mucus (MUC), smooth muscle (MUS), normal colon mucosa (NORM), cancer-associated stroma (STR), colorectal adenocarcinoma epithelium (TUM). Scripts for reproducing the inference results can be found at world wide website github.com/labsyspharm/orion-crc.

### EXAMPLE XV

### Outcome Analysis

Outcome analysis was performed using Kaplan-Meyer estimation and log-rank test utilizing the MatSurv function in MATLAB, see e.g., Creed, J. H., Gerke, T. A. & Berglund, A. E. MatSurv: Survival analysis and visualization in MATLAB. Journal of Open Source Software 5, 1830 (2020). Cutoffs for IFM1, IFM2, and IFM3 were selected at the median value of the entire cohort, and cutoff for IFM4 were selected based on IFM1 & IFM3 as described. Hazard ratios and confidence intervals were calculated with the log-rank approach: HR=(Oa/Ea)/(Ob/Eb), where Oa & Ob are the observed events in each group and Ea & Eb are the number of expected events.

### EXAMPLE XVI

### Platforms for Immunofluorescence in a Immunohistochemistry Assay

The present disclosure provides immunohistochemistry methods including immunostaining. According to one aspect, pan-cytokeratin and E-cadherin antigens are identified in cells of a tissue section by exploiting the principle of antibodies binding specifically to pan-cytokeratin and E-cadherin antigens in biological tissues. The antibodies can be labeled directly or indirectly as is known in the art.

According to certain aspects, the present disclosure provides materials, instruments and methods to carry out immunofluorescence methods of a colorectal tissue sample using detectable agents, such as detectable antibodies. Immunofluorescence materials, instruments and methods are known. One exemplary imaging assay includes the 5 to 6-plex imaging of tissue sections using a Perkin Elmer Vectra Polaris™ (now Akoya Phenolmager HT™) combined in some cases with H&E imaging of adjacent sections, see e.g., O'Meara, T. A. et al. Abstract P1-04-05: Multiplexed immunofluorescence staining of intra-tumoral immune cell populations and associations with immunohistochemical, clinical, and pathologic variables in breast cancer. Cancer Research 82, P1-04-05 (2022); Berry, S. et al. Analysis of multispectral imaging with the AstroPath platform informs efficacy of PD-1 blockade. Science 372, eaba2609 (2021). One exemplary imaging assay includes the ORION method and a commercial-grade instrument that implements it, to established IHC and cyclic data acquisition by CyCIF, see e.g., Lee, S. et al. Novel charged sodium and calcium channel inhibitor active against neurogenic inflammation. Elife 8, e48118 (2019).

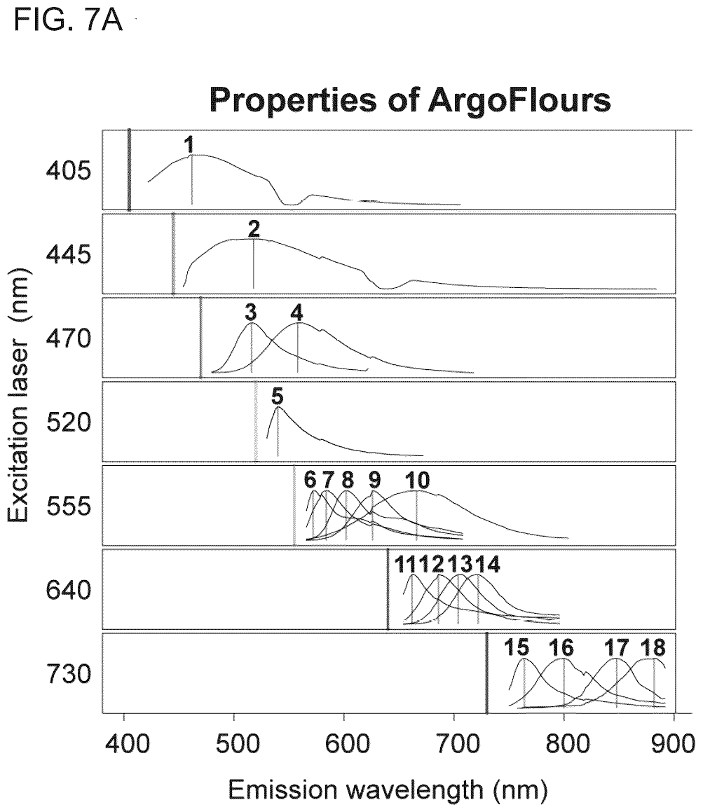

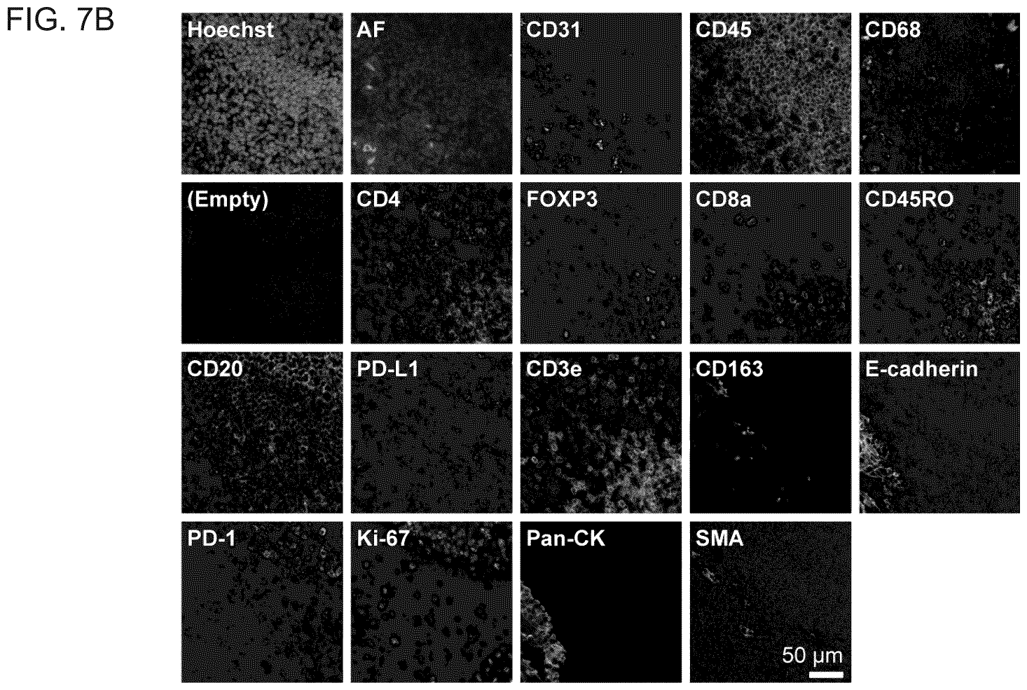

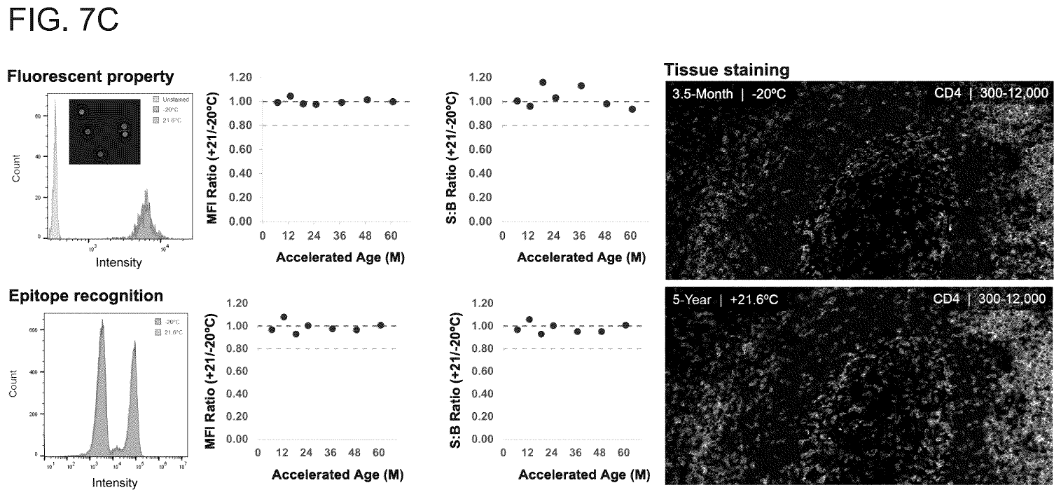

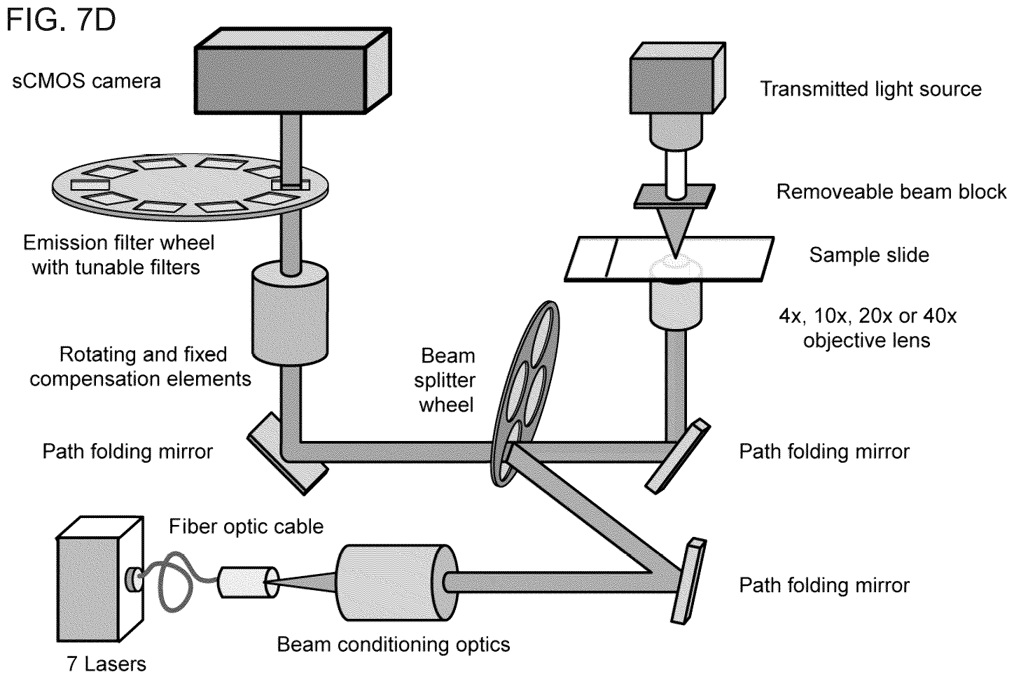

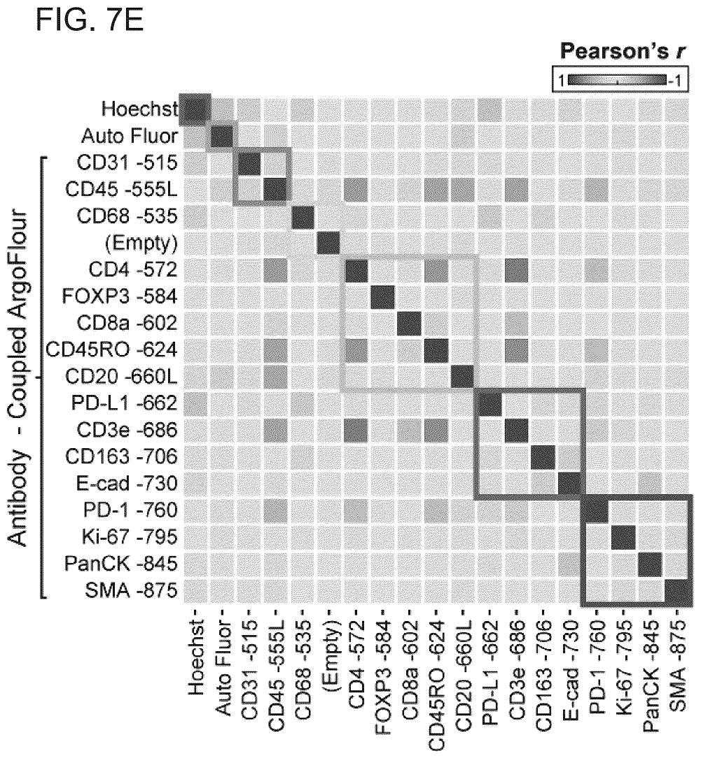

According to one aspect, dyes useful in the present disclosure for IF methods, such as ArgoFluor™ dyes, are compatible with spectral extraction by discrete sampling based on the following properties: (i) emission in the 500-875 nm range; (ii) high quantum-efficiency; (iii) good photostability; and (iv) compatibility with each other in high-plex panels (FIG. 7A, Extended Data Table 2, FIG. 14). ArgoFluor dyes were covalently coupled to commercial antibodies directed against lineage markers of immune (e.g., CD4, CD8, CD68), epithelial (cytokeratin, E-cadherin), and endothelial (CD31) cells as well as immune checkpoint regulators (PD-1, PD-L1), and cell state markers (Ki-67), to generate panels suitable for studying the microenvironment and architecture of epithelial tumors and adjacent normal tissue (FIG. 7B). An accelerated aging test demonstrated excellent reagent stability, estimated to be >5 yr at −20° C. storage (FIG. 7C).

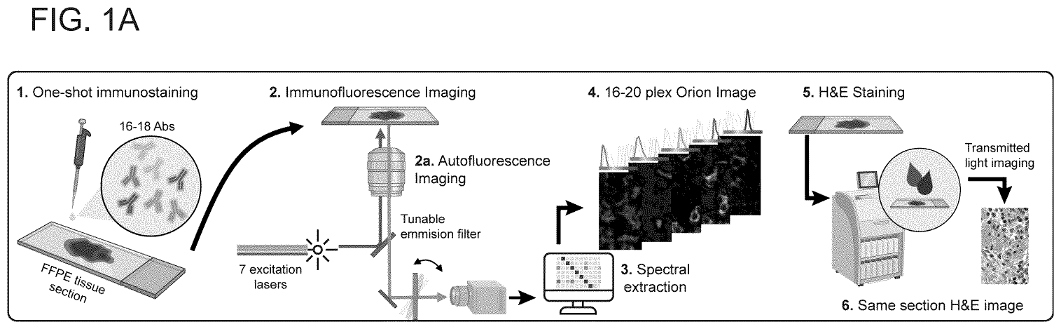

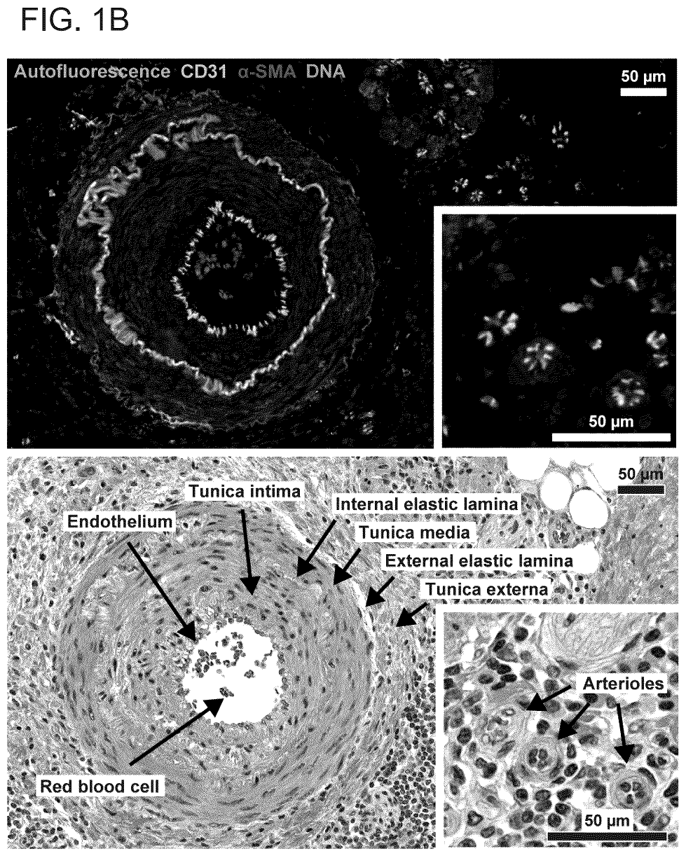

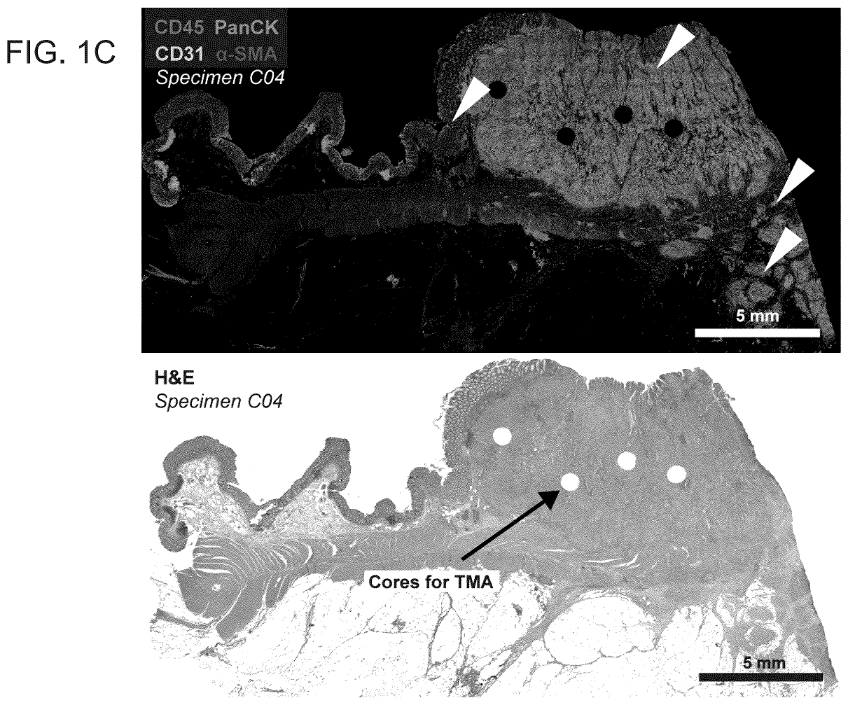

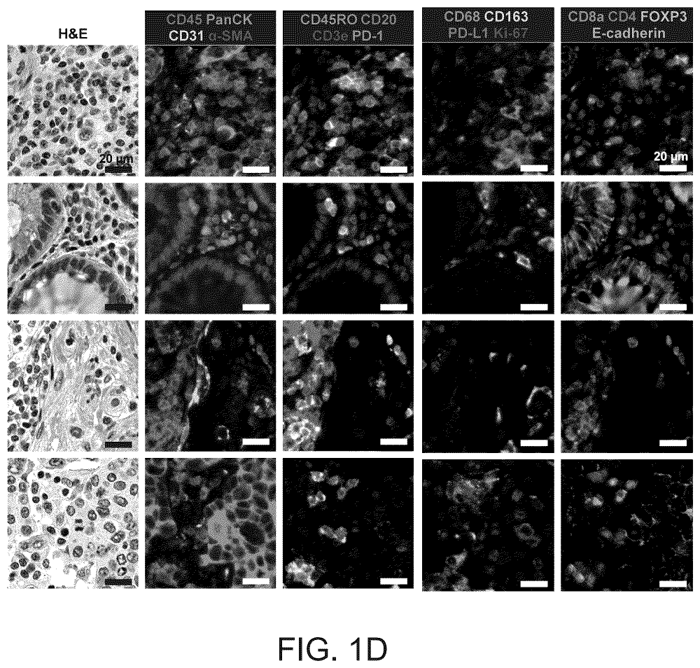

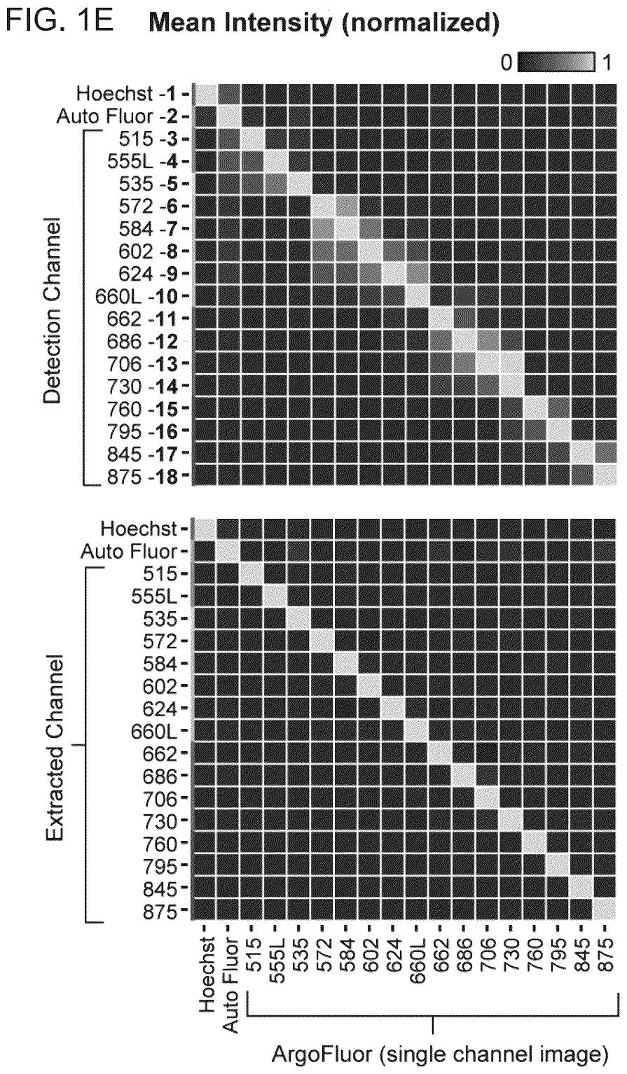

In order to identify pan-cytokeratin and E-cadherin as markers of progression free survival of colorectal cancer, many markers were studied along with pan-cytokeratin and E-cadherin using data generated by the ORION system. A commercial-grade Orion instrument was developed. The instrument utilizes seven lasers (FIG. 1A and FIG. 7D) to illuminate the sample and collect the emitted light with 4× to 40× objective lenses (0.2 NA to 0.95 NA). The system employs multiple tunable optical filters, see e.g., Favreau, P. et al. Thin-film tunable filters for hyperspectral fluorescence microscopy. J Biomed Opt 19, 011017 (2014), that use a non-orthogonal angle of incidence on thin-film interference filters to shift the emission bandpass, see e.g., Anderson, N., Beeson, R. & Erdogan, T. Angle-Tuned Thin-Film Interference Filters for Spectral Imaging. Optics and Photonics News 13, 1-2 (2011). These filters have 90-95% transmission efficiency and enable collection of 10-15 nm bandpass channels with 1 nm center wavelength (CWL) tuning resolution over a wide range of wavelengths (425 to 895 nm). Narrow bandpass emission channels improve specificity and the consequent reduction in signal strength is overcome by using excitation lasers that are ˜10 times brighter than conventional LED illuminators and a sensitive scientific CMOS detector. Raw image files are processed computationally to correct for system aberrations such as geometric distortions and camera non-linearity, see e.g., Zeng, Z. et al. Computational methods in super-resolution microscopy. Frontiers Inf Technol Electronic Eng 18, 1222-1235 (2017), followed by spectral extraction to remove crosstalk, thereby isolating individual biomarker signals to one per imaging channel. The features of single-cells and regions of tissue are then computed using MCMICRO software, see e.g., Schapiro, D. et al. MCMICRO: a scalable, modular image-processing pipeline for multiplexed tissue imaging. Nat Methods 19, 311-315 (2022). The Orion instrument has an integrated brightfield mode, but the H&E images used in the study were also acquired using an Aperio GT450 microscope (Leica Biosystems), which is a gold standard for diagnostic applications, see e.g., Babawale, M. et al. Verification and Validation of Digital Pathology (Whole Slide Imaging) for Primary Histopathological Diagnosis: All Wales Experience. J Pathol Inform 12, 4 (2021) (FIG. 1A).

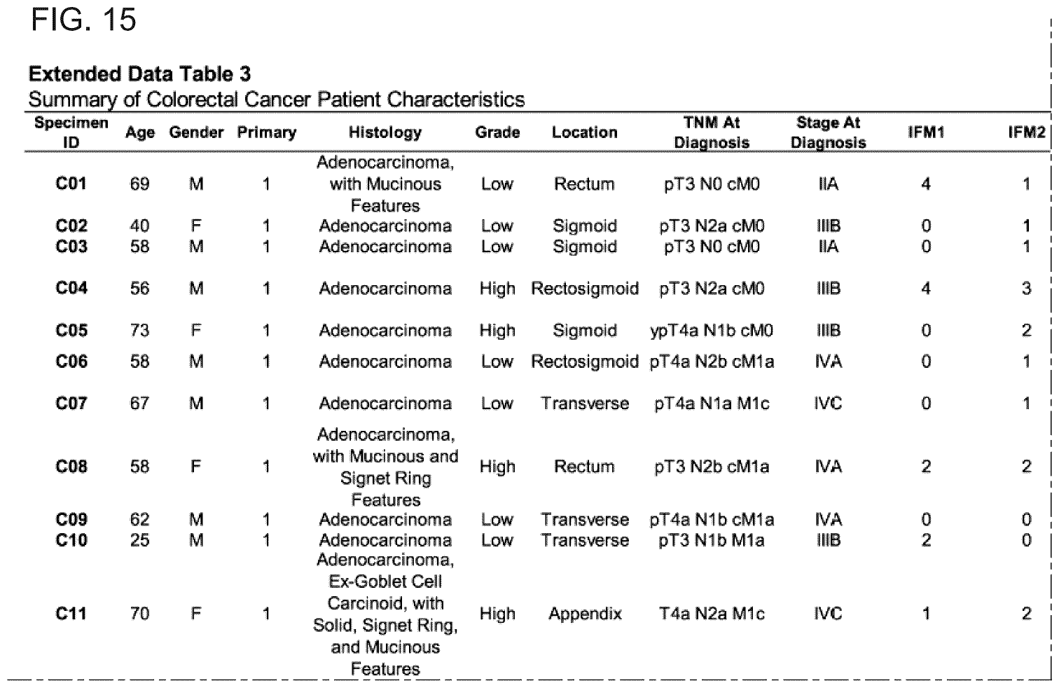

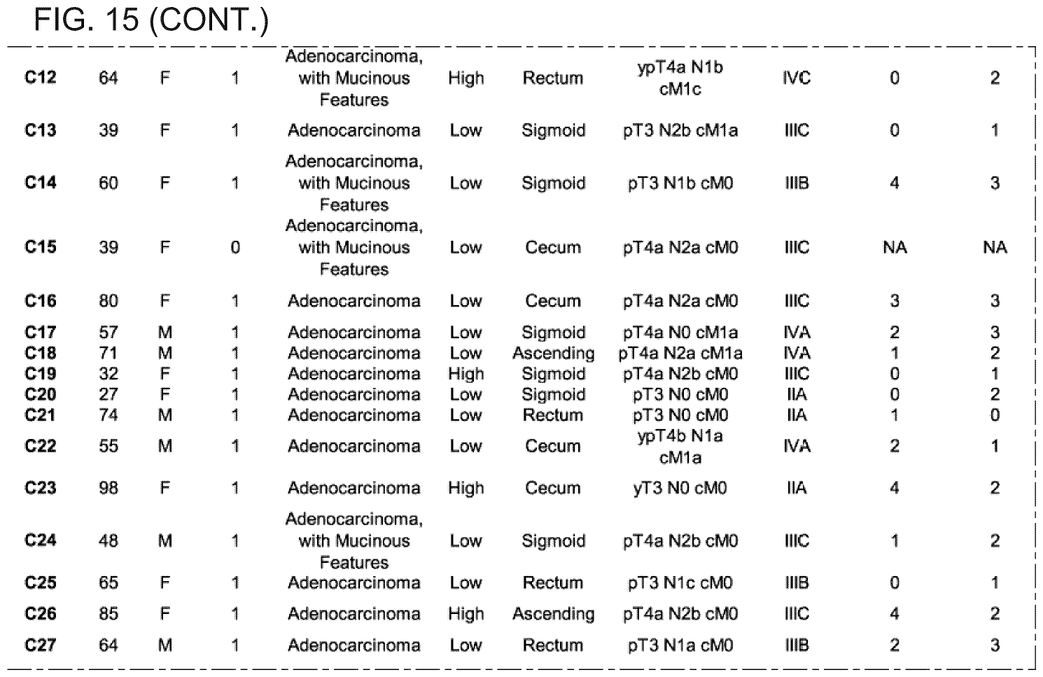

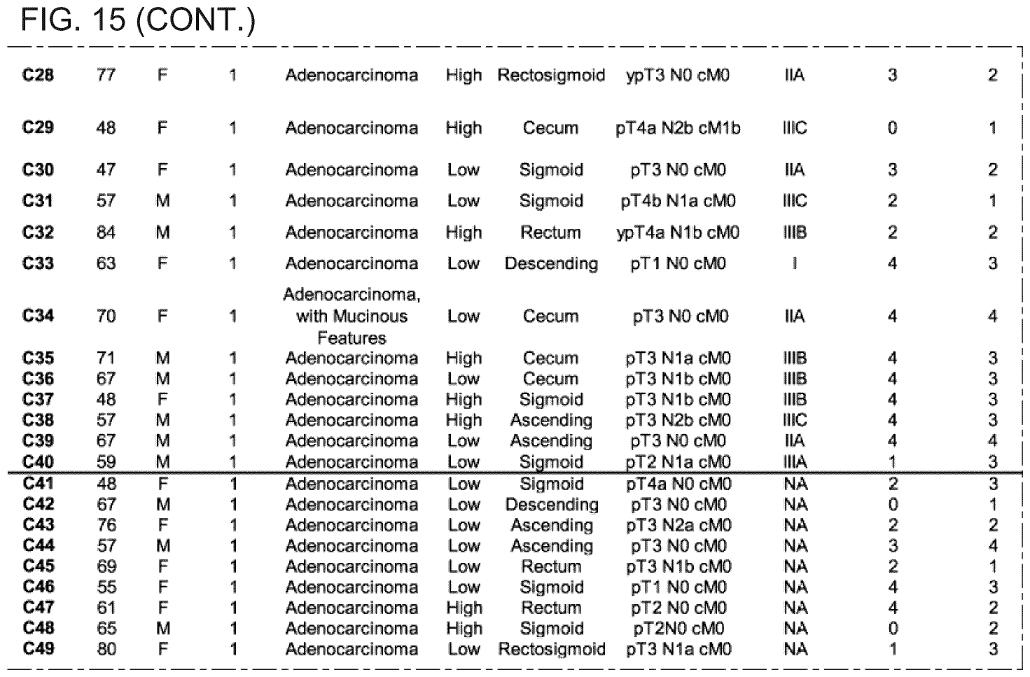

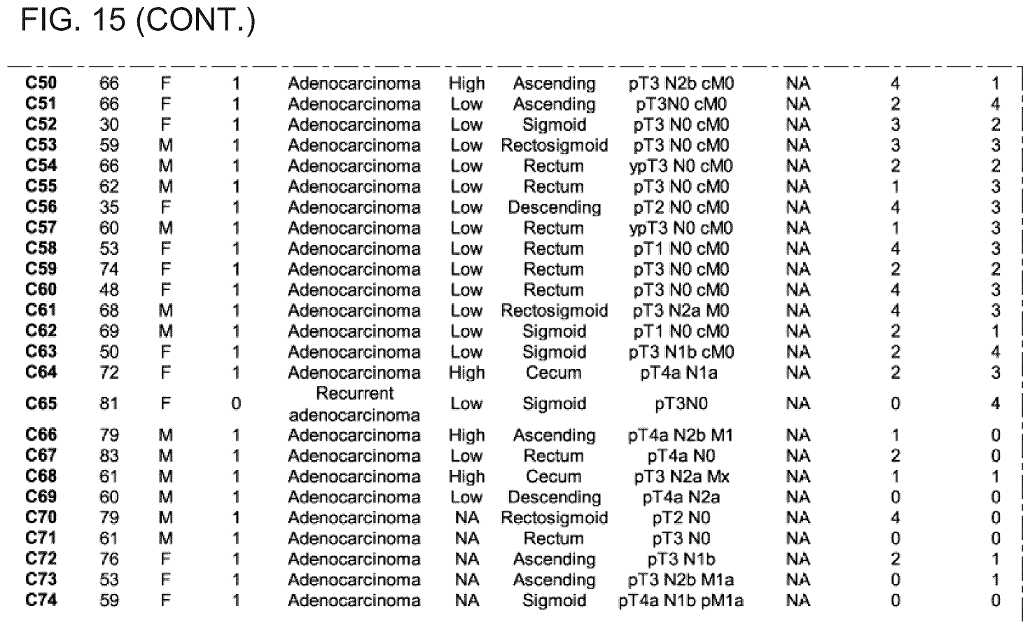

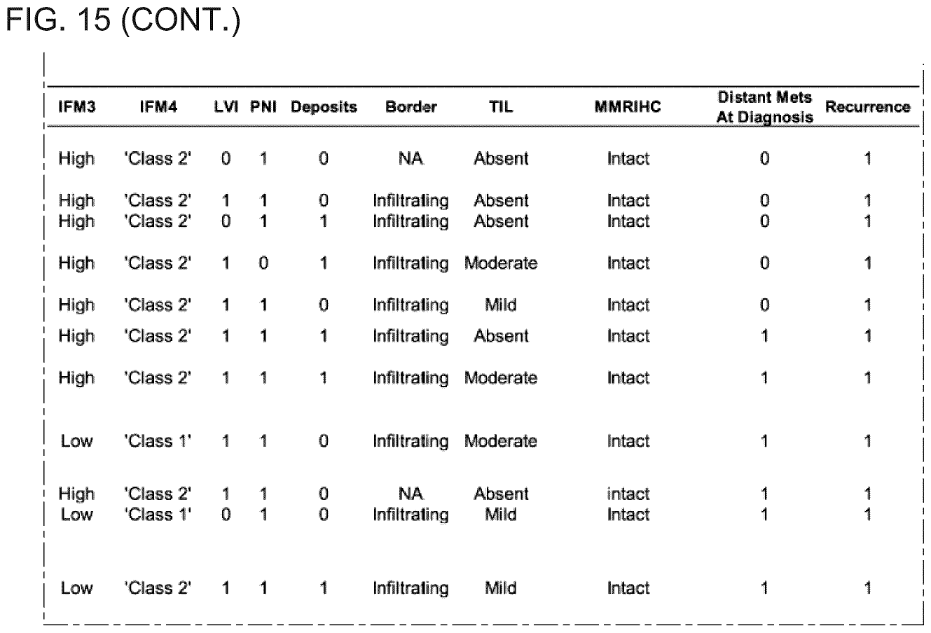

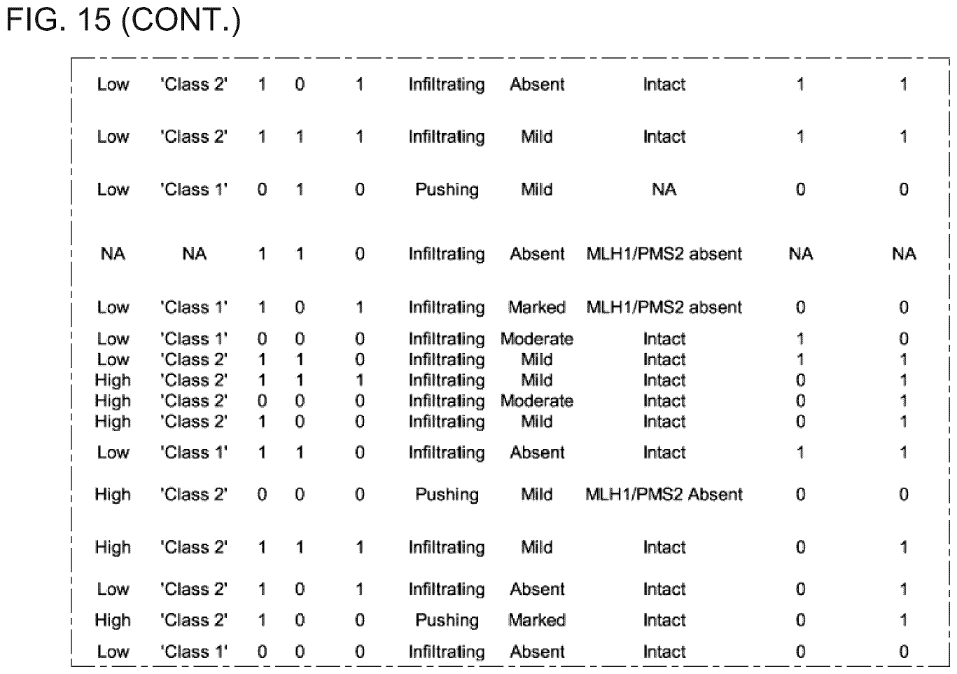

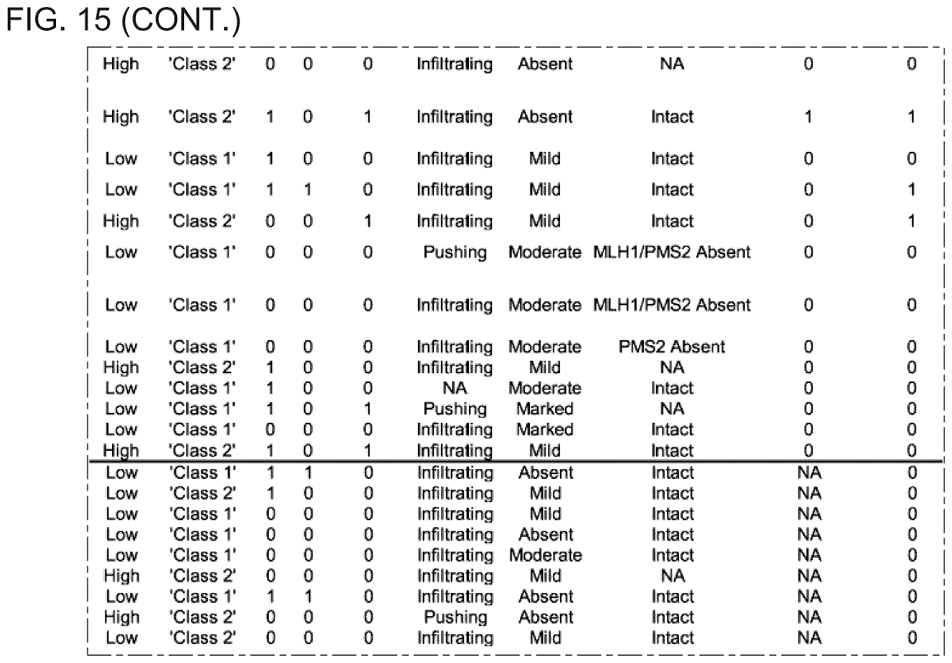

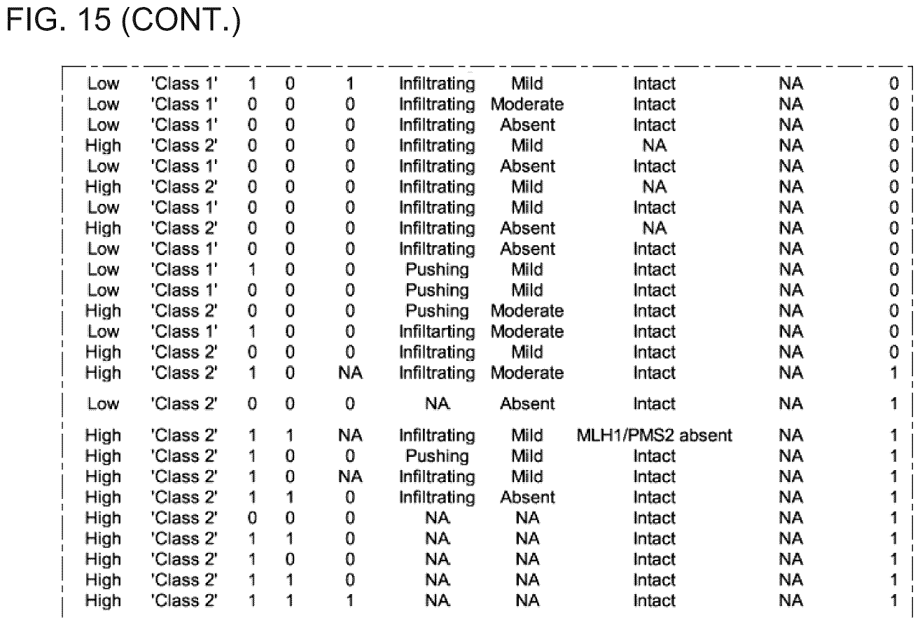

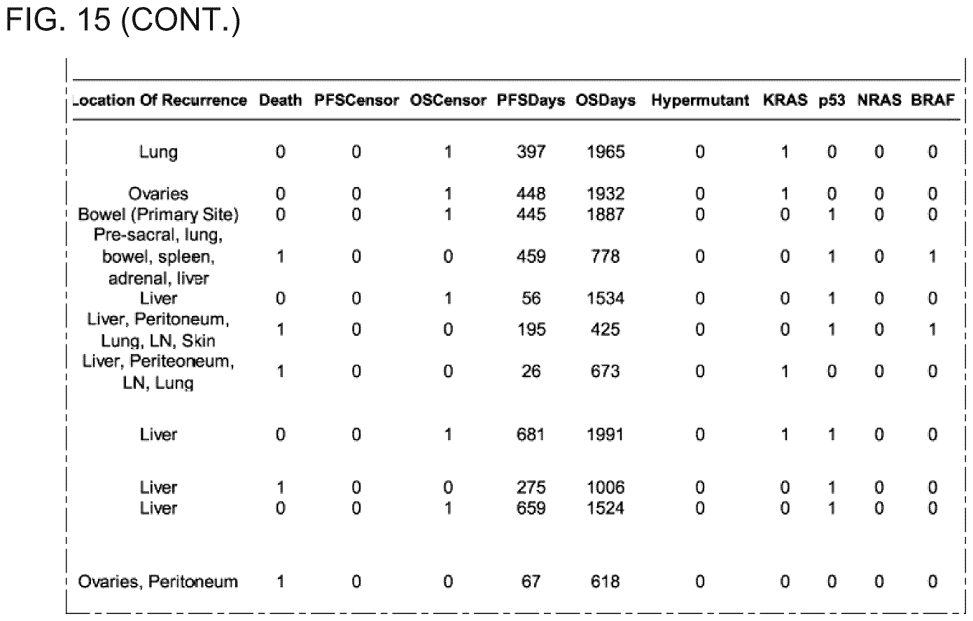

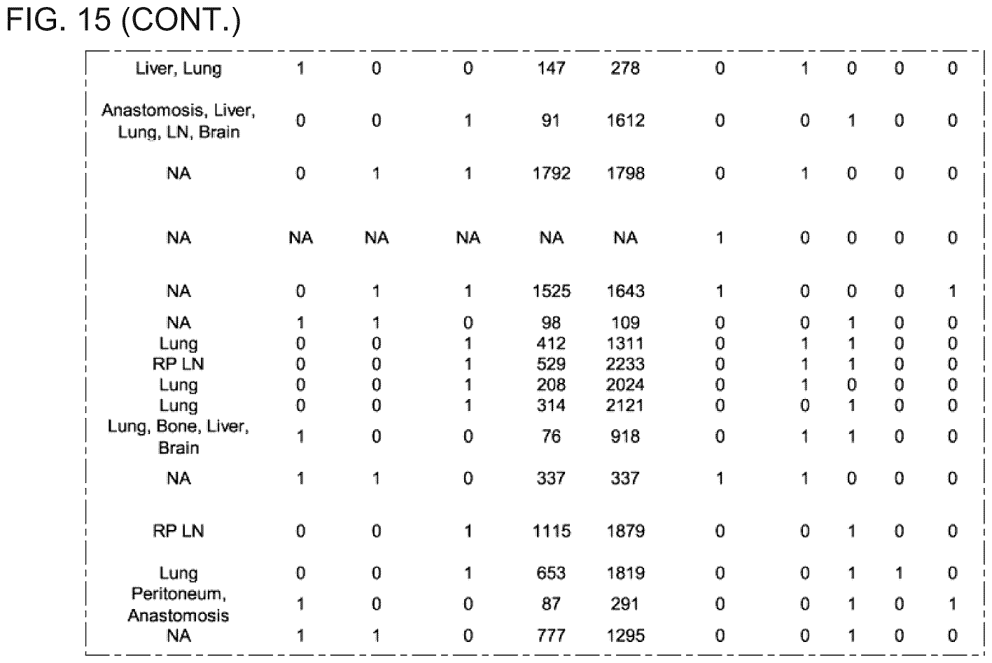

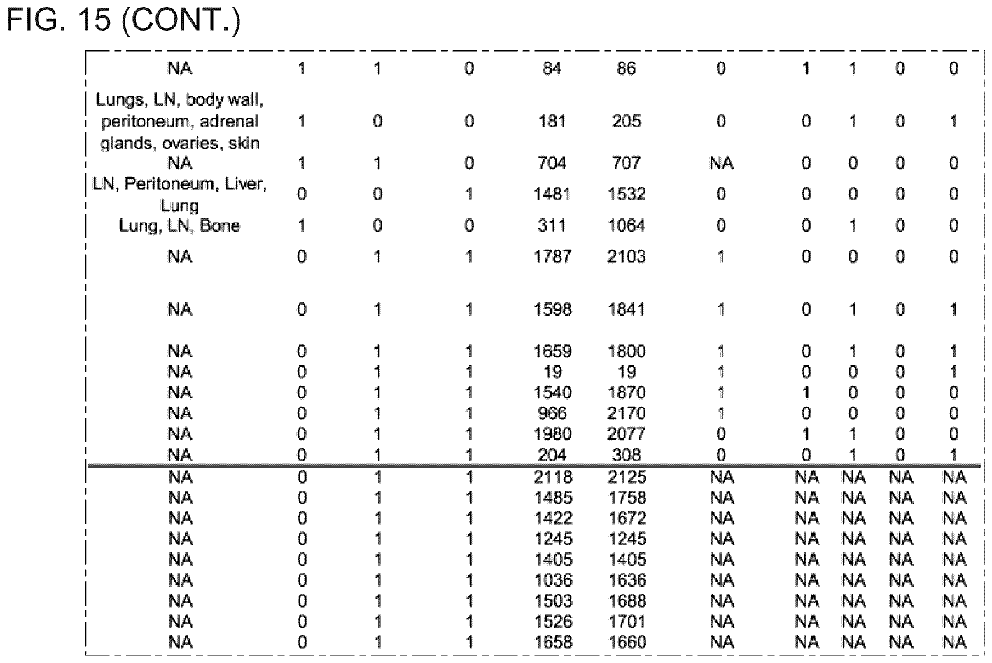

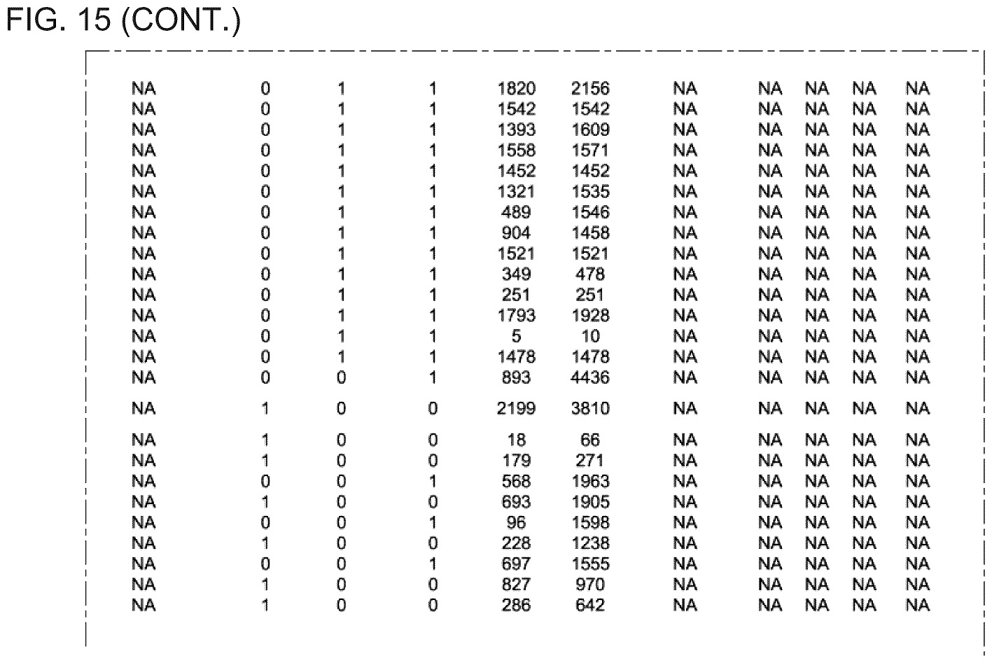

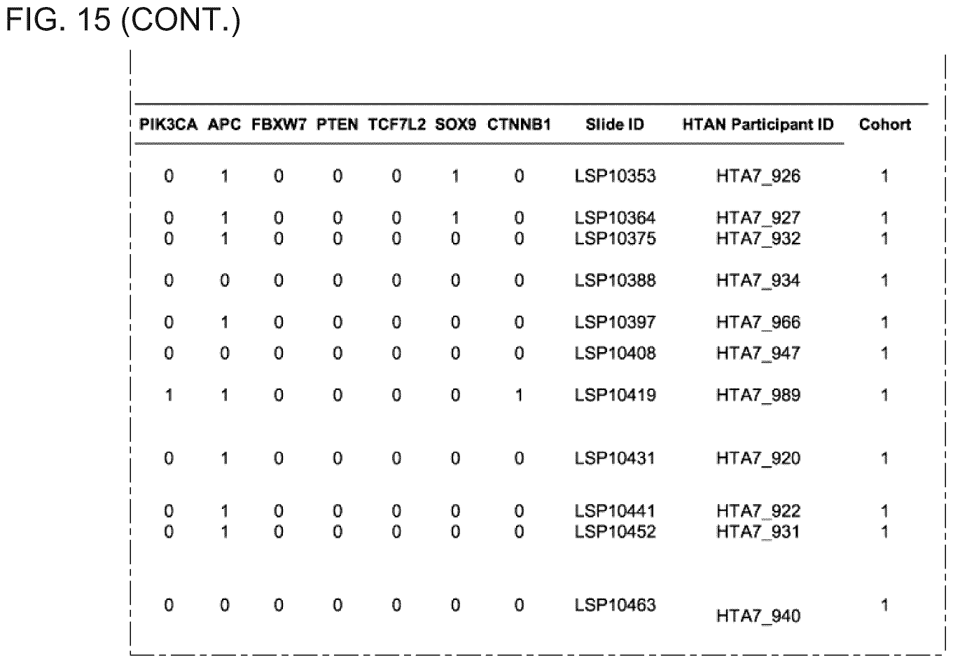

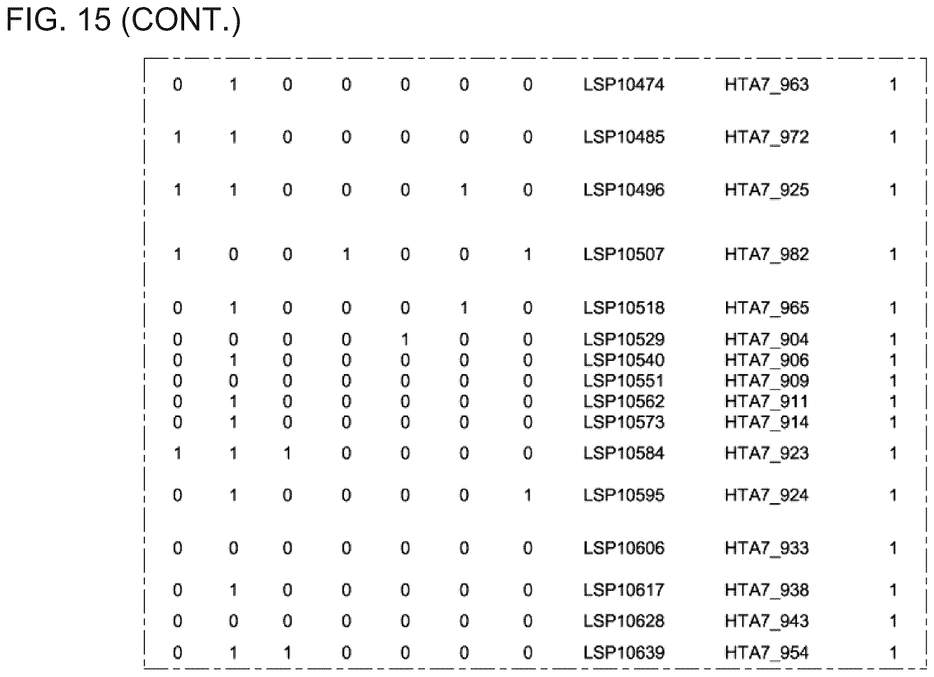

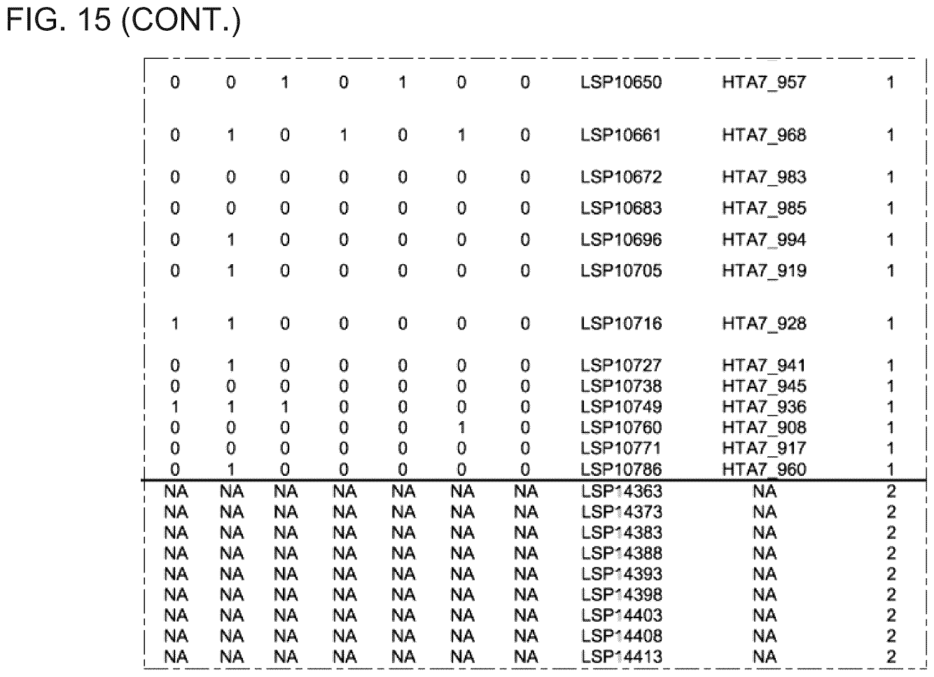

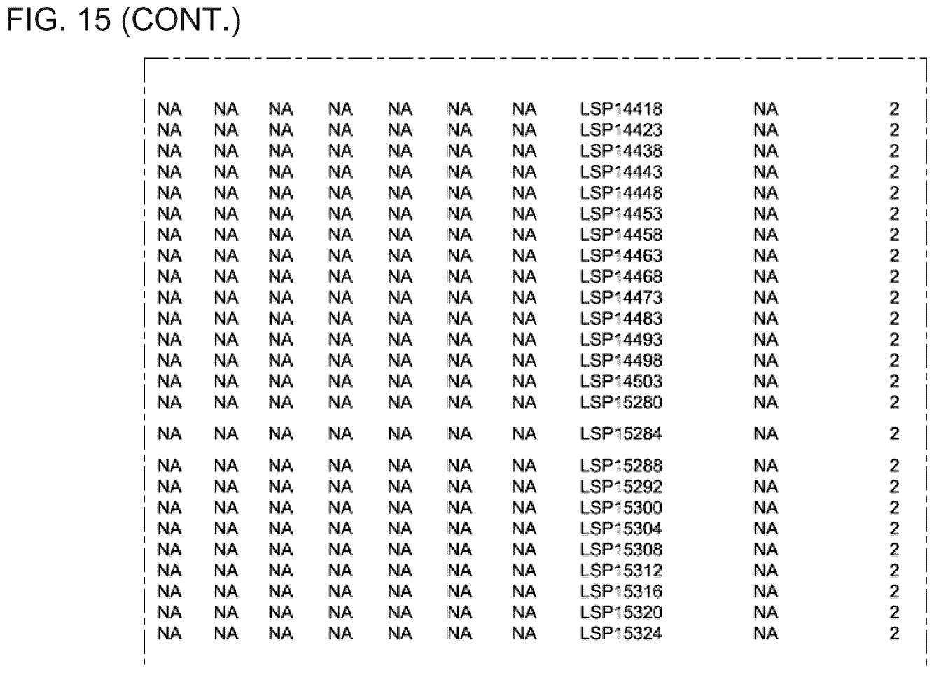

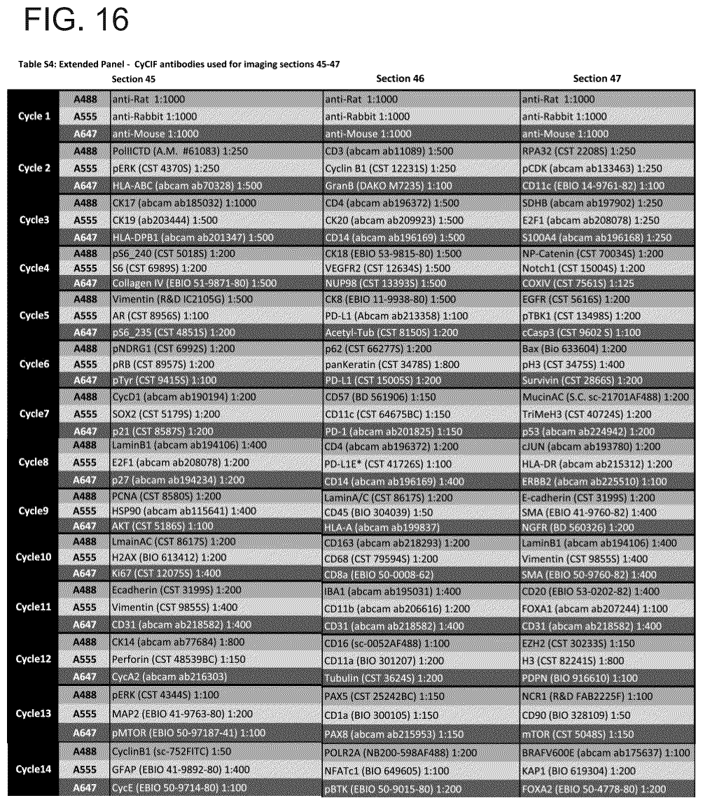

Colorectal cancer tissue was tested along with other tissue types as a comparison of results to confirm utility. Images from three sets of FFPE specimens were collected (i) human tonsils, a standard tissue for antibody qualification, (ii) 40 stage I-IV colorectal cancer (CRC) resections from the archives of the Brigham and Women's Hospital Pathology Department (key features of this cohort are described in Extended Data Table 3, FIG. 15), and (iii) specimens of multiple tumor types available on TMA (Extended Data Table 4, FIG. 16). The panel on tonsil was optimized and applied to the colorectal cancer cohort and to other tissue types represented on the TMA. A dedicated autofluorescence channel (445 nm excitation/485 nm emission, CWL) was included that provided valuable information on tissue morphology and components of connective tissue structures and blood vessels (FIG. 1B), see e.g., STEINER, K. Fluorescence Microscopy of Normal and Pathologic Keratin. Archives of Dermatology 82, 352-361 (1960). This channel was also used to extract autofluorescence from the IF channels and improve biomarker signal to noise ratio (SNR). The images obtained represent 18-plex imaging (16 antibody channels, autofluorescence and nuclear stain) plus H&E. Inspection of extracted images revealed error-free whole-slide imaging of 1,000 or more adjacent tiles (FIG. 1C), see e.g., Muhlich, J., Chen, Y. A., Russell, D. & Sorger, P. K. Stitching and registering highly multiplexed whole slide images of tissues and tumors using ASHLAR software. (2021) doi: 10.1101/2021.04.20.440625, as well as bright infocus staining of cellular and cellular substructures within each tile (FIG. 1D). To quantify the effectiveness of spectral extraction, serial sections of human tonsil each stained with an individual antibody conjugated to a different ArgoFluor fluorophore were imaged and then data in all channels was recorded. Prior to extraction, spectral cross talk between adjacent channels averaged ˜35% and this was reduced 35-fold to <1% following spectral extraction (FIG. 1E; crosstalk among all channel pairs was reduced to <0.5%). When a tissue section was subjected to one-shot 16-plex antibody labeling, “cross-talk” was observed only for antibodies that stain targets co-localized on the same types of cells (e.g., co-staining of T-cell membranes by antibodies against CD3e and CD4 resulted in a high degree of pixel intensity correlation across these two channels; FIG. 7E).

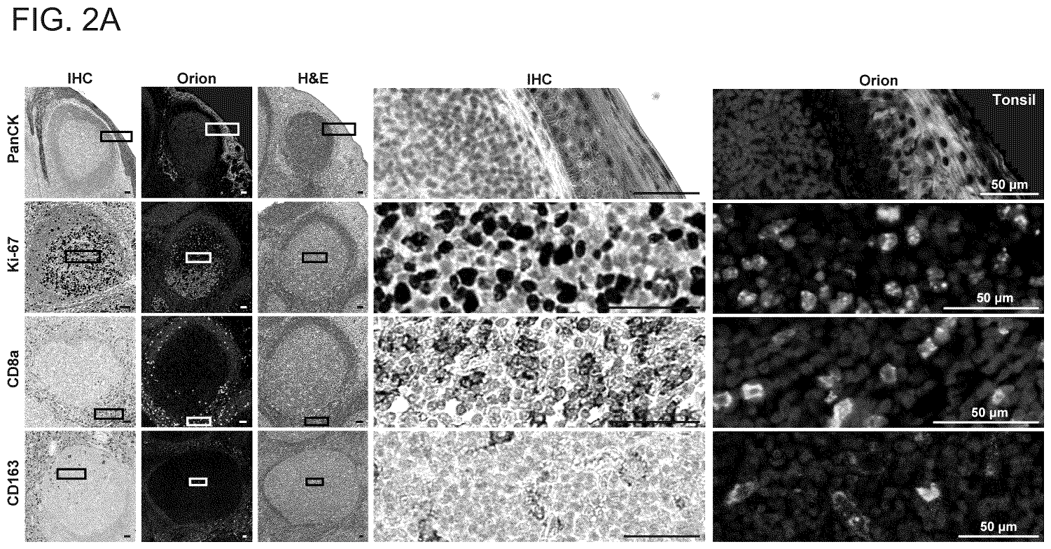

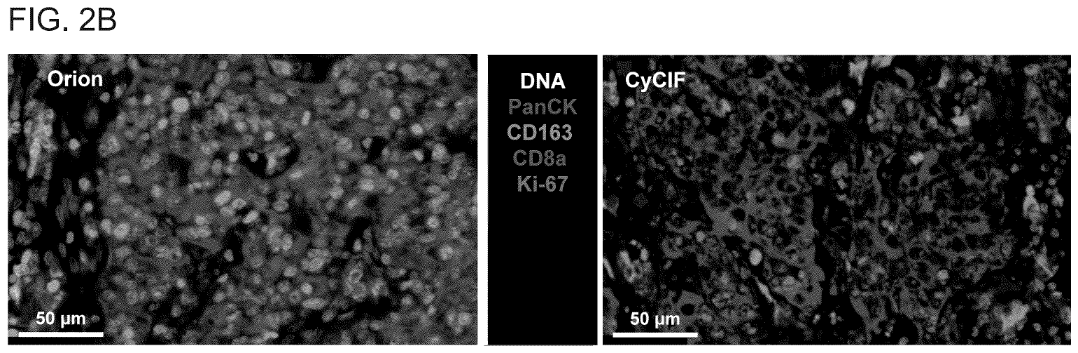

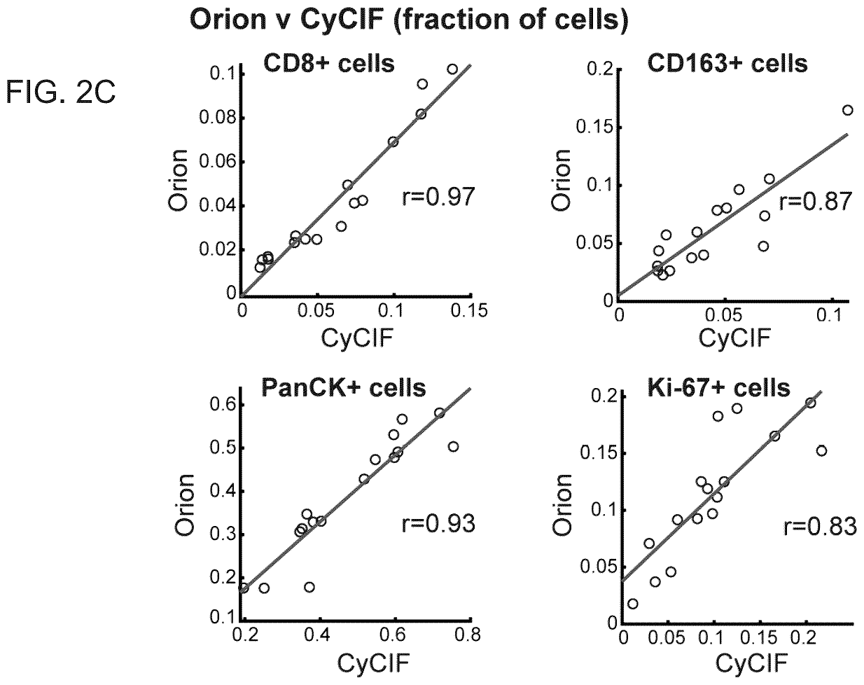

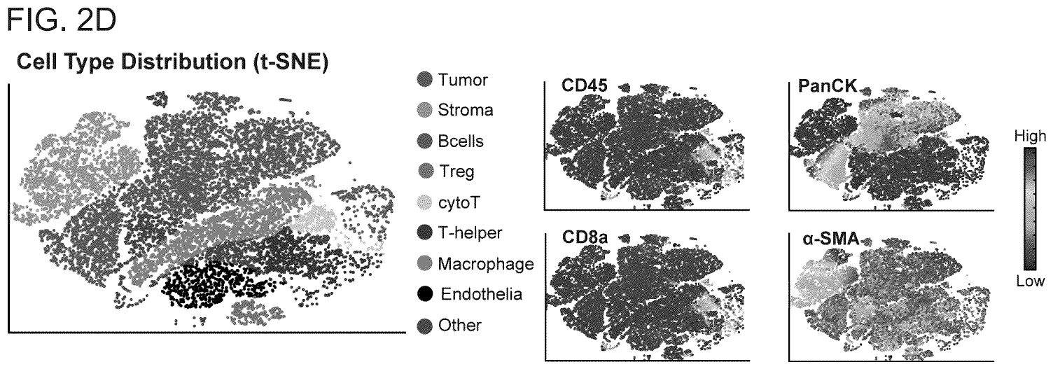

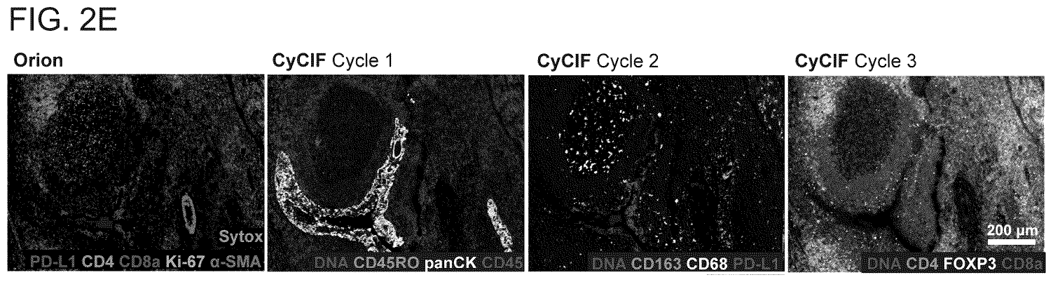

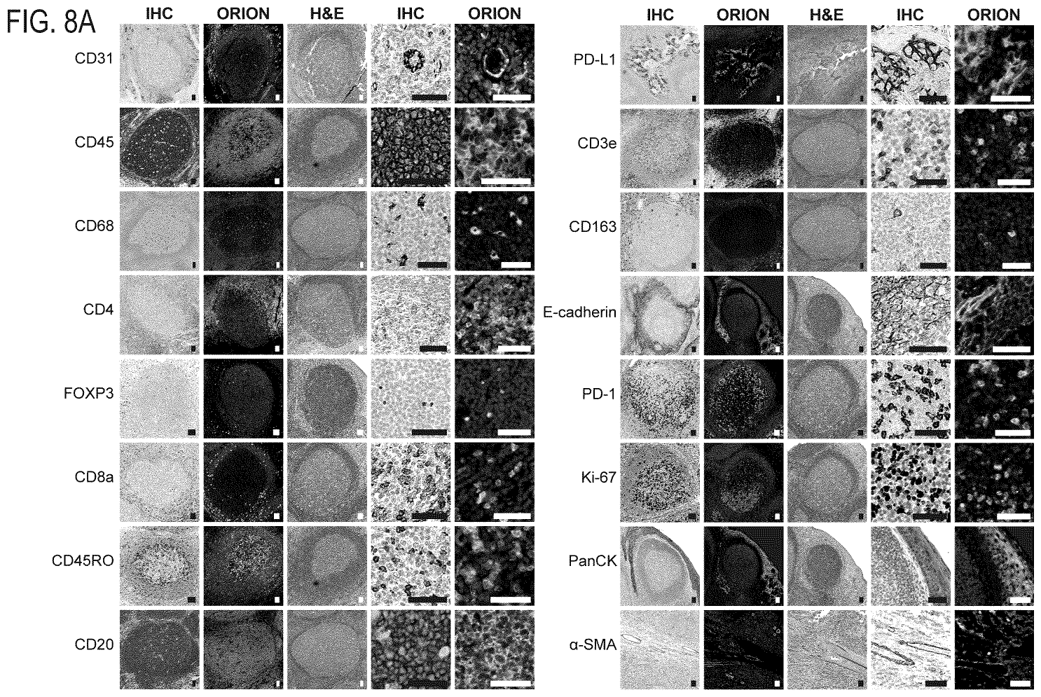

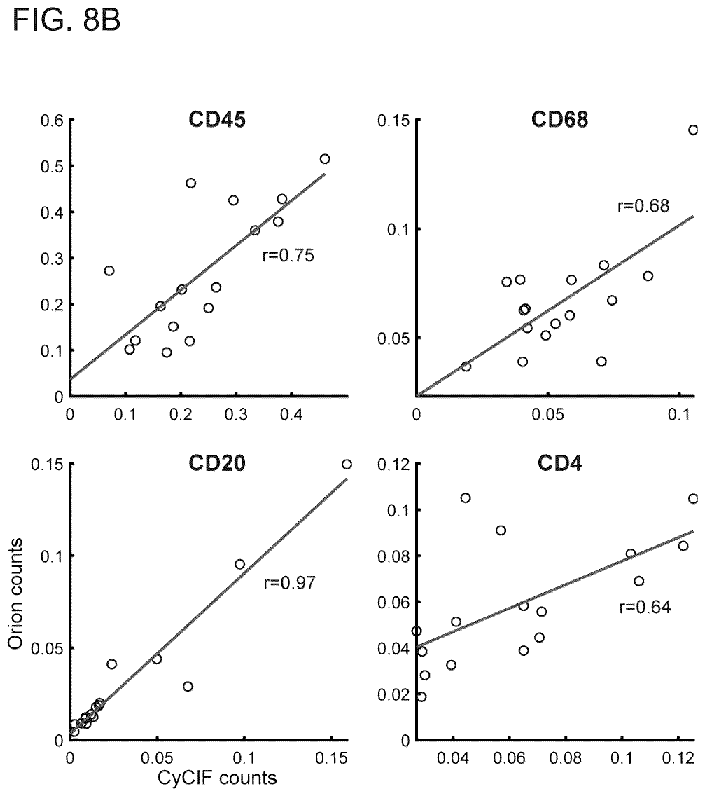

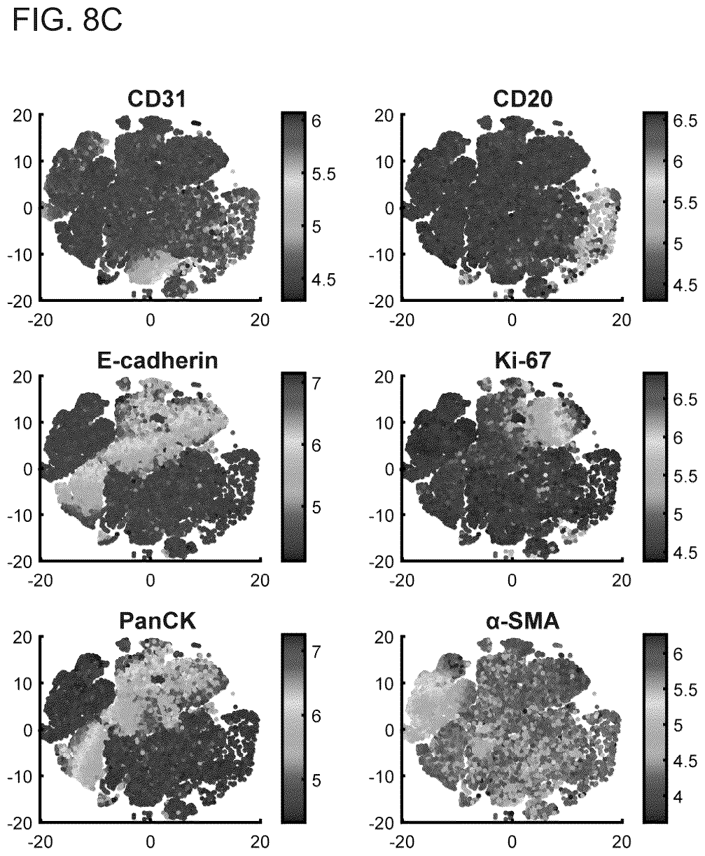

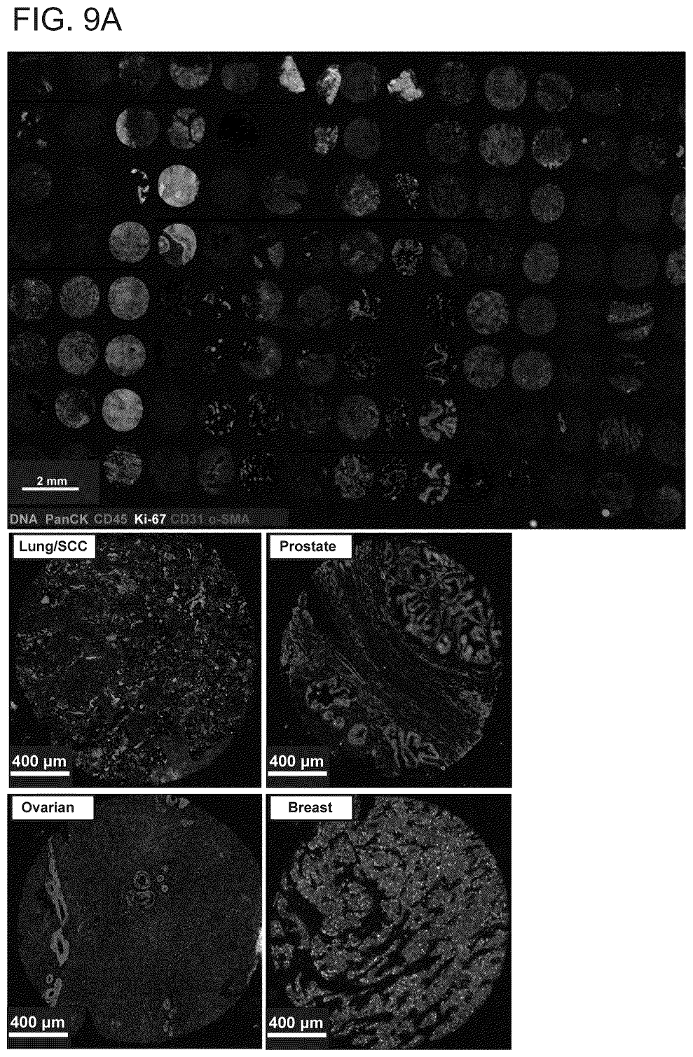

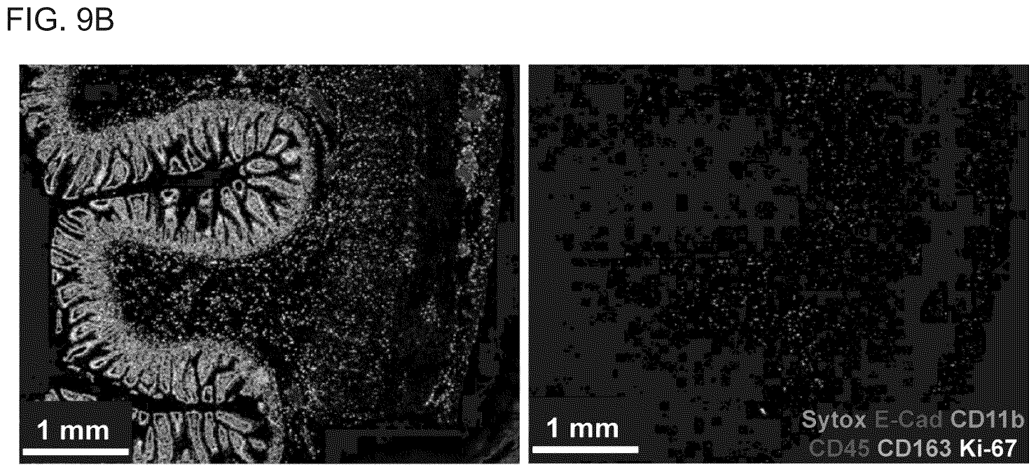

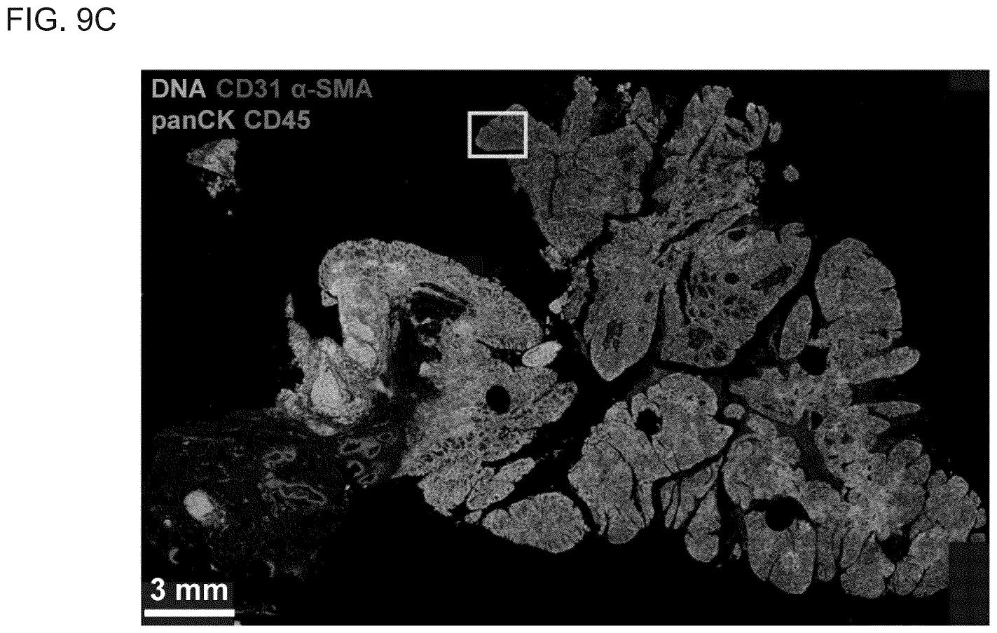

The staining patterns obtained by ArgoFluor-antibody conjugates were similar to those obtained by conventional IHC performed on the same specimen using the same antibody clones, see e.g., Du, Z. et al. Qualifying antibodies for image-based immune profiling and multiplexed tissue imaging. Nat Protoc 14, 2900-2930 (2019), for details of approach; FIG. 2A and FIG. 9A-9C). In addition, when adjacent tissue sections from colorectal cancer patients were imaged using the ORION method and apparatus and the well-established method of cyclic immunofluorescence (CyCIF), see e.g., Lin, J. R. et al. Highly multiplexed immunofluorescence imaging of human tissues and tumors using t-CyCIF and conventional optical microscopes. Elife 7, e31657 (2018), images looked similar and the fractions of cells scoring positive for identical markers were highly correlated (FIG. 2B, 2C and FIG. 8B shows four examples with p=0.8 to 0.9). Furthermore, projection of the high dimensional Orion data using t-SNE successfully resolved multiple immune and tumor cell types (FIG. 2D and FIG. 8C). Accordingly, the ORION method, IHC and CyCIF are all useful immunofluorescence methods for identifying pan-cytokeratin and E-cadherin as markers for colorectal cancer in tissue samples and cells.

According to one aspect, immunofluorescence methods can be combined to increase the number of fluorescent antibodies for a desired application. For example, CyCIF is performed after Orion imaging (but prior to H&E staining). The standard CyCIF signal reduction (“bleaching”) procedure, see e.g., Lin, J. R. et al. Highly multiplexed immunofluorescence imaging of human tissues and tumors using t-CyCIF and conventional optical microscopes. Elife 7, e31657 (2018), reduced ArgoFluor intensity by over 95% on average (FIG. 9B), enabling the collection of multiple rounds of multiplexed imaging data after the original Orion imaging round (FIG. 2E), if desired.

### EXAMPLE XVII

### Integrated Analysis of IF and H&E Images

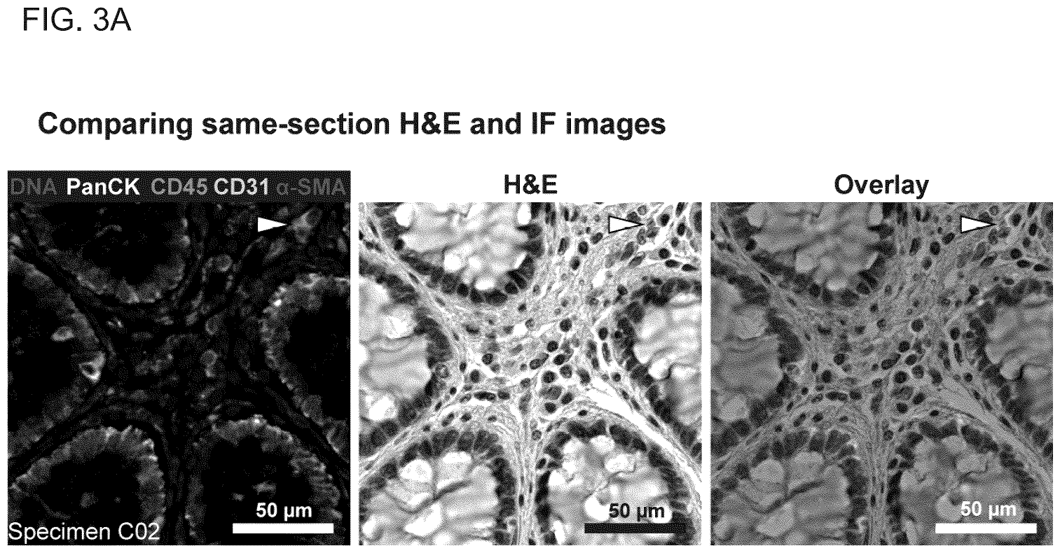

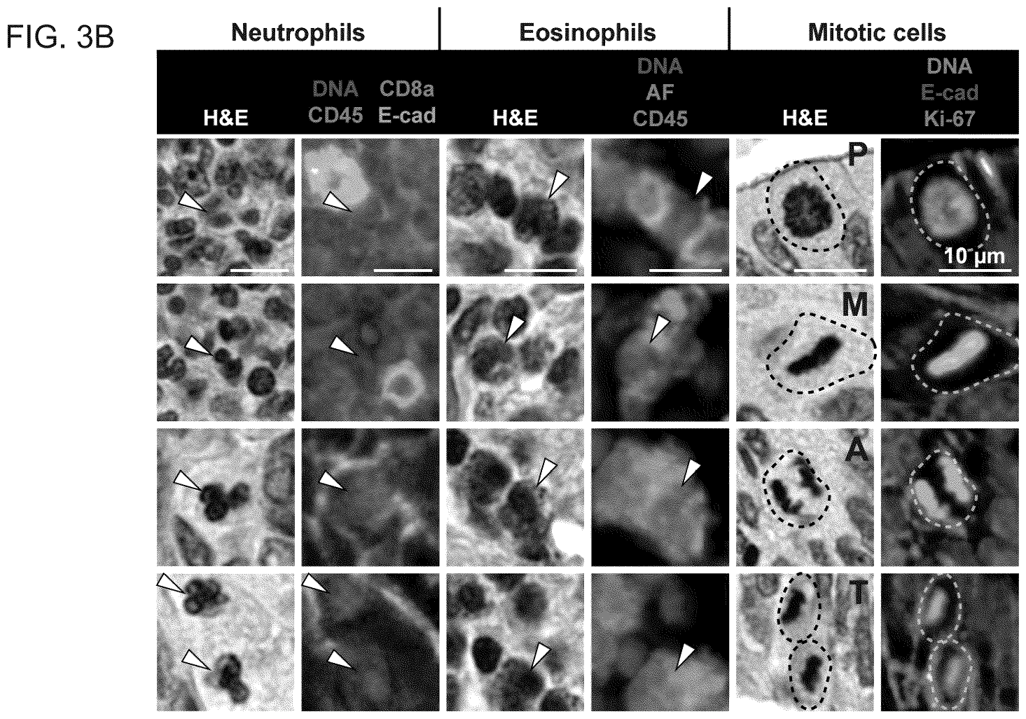

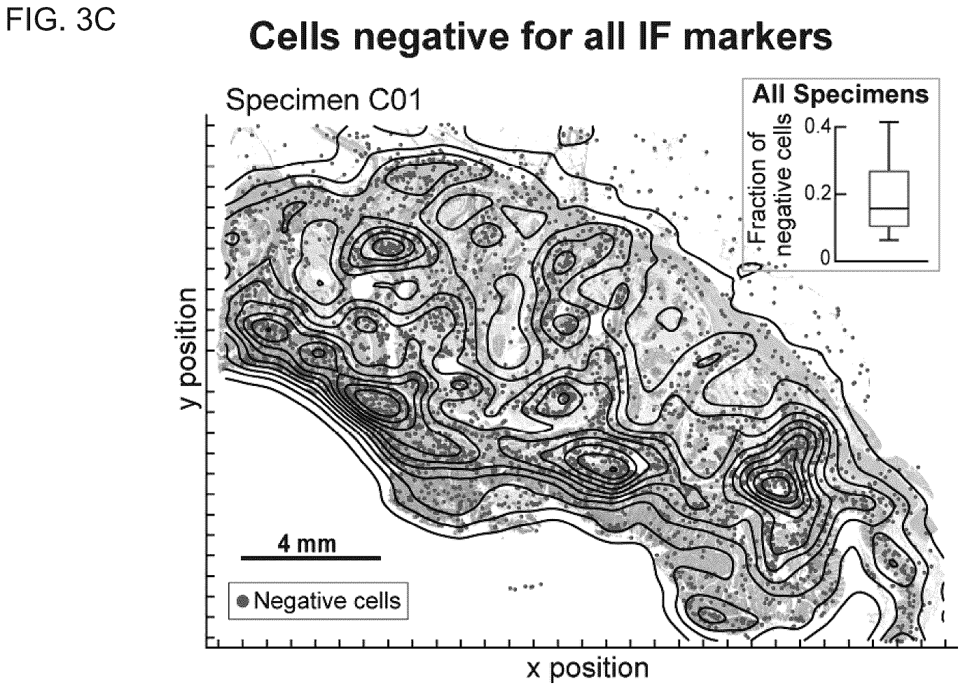

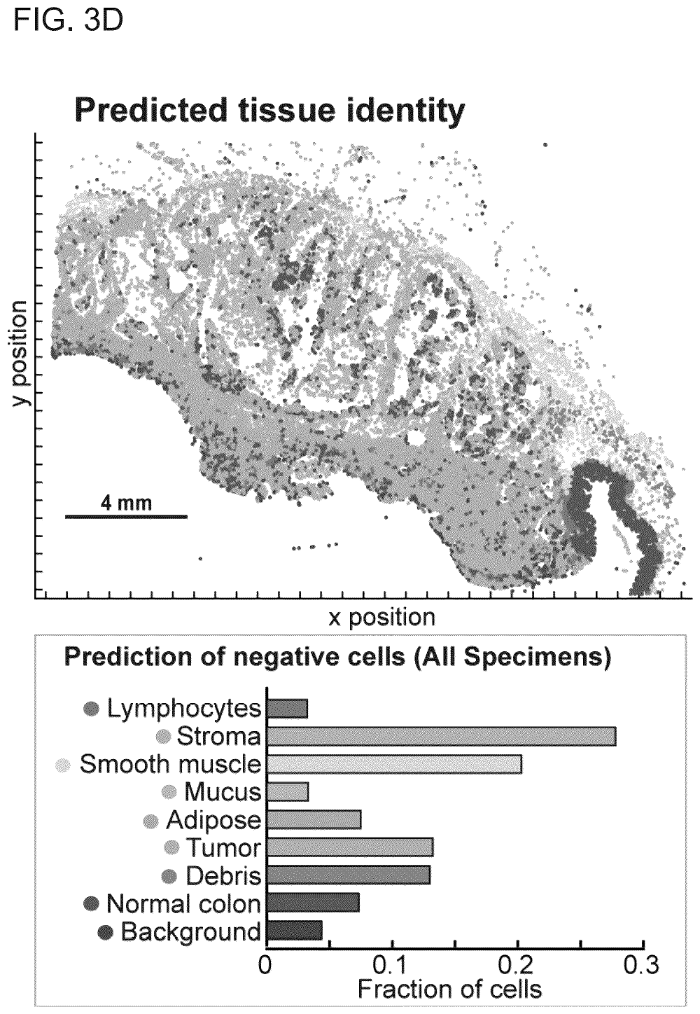

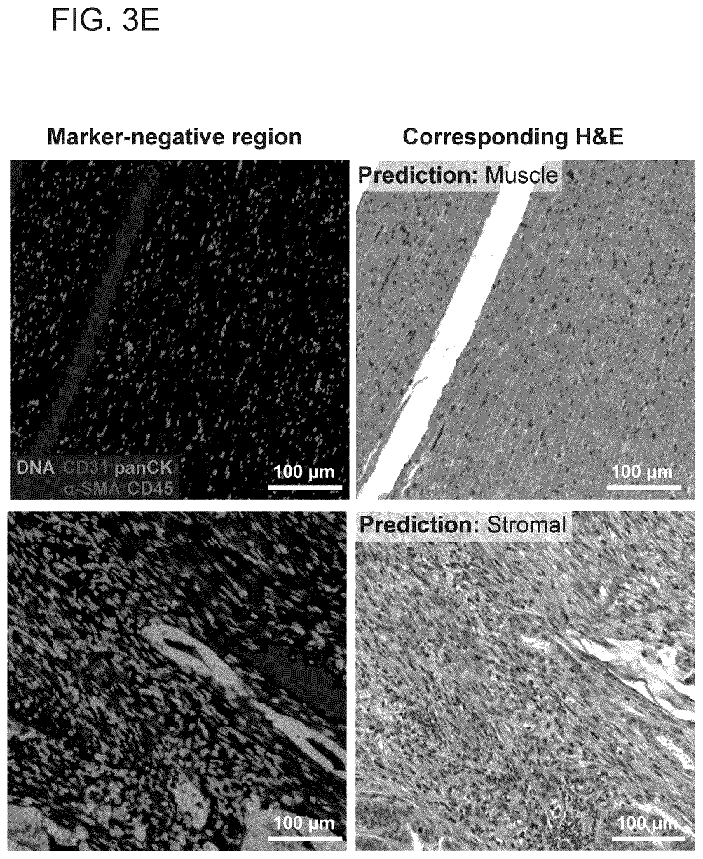

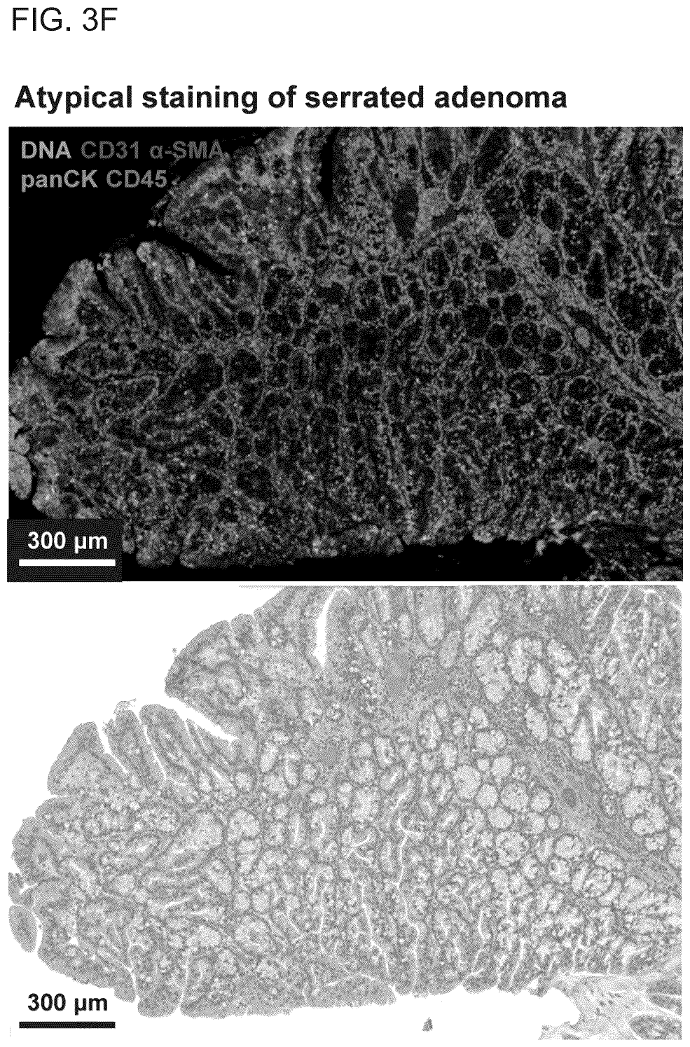

The Orion method enables same-cell H&E and IF comparison (FIG. 3A), as opposed to existing methods that require use of adjacent tissue sections. Molecular labels obtained from IF enabled more complete enumeration of lymphocytes than inspection of H&E images by trained pathologists alone; for example, cells in CD4, CD8 T cell and B cell lineages are similar by H&E but clearly distinguishable by IF (arrows in FIG. 3A). Conversely, some cells and cell states were more readily defined in H&E images based on morphologic features than by immunofluorescence markers; this included eosinophils, neutrophils that could not be subtyped in IF images but whose morphology is highly characteristic in H&E data, as well as the prophase, metaphase, anaphase and telophase stages of mitosis (arrows and dashed lines in FIG. 3B). To quantify the amount of complementary information in H&E and IF images, the number of cells (as identified by nuclear segmentation) in the 40-specimen colorectal cancer dataset that could not be assigned a clear identity using IF images were computed and found that the number of cells varied from 6.5 to 42% of total nuclei (median=16%) (FIG. 3C). A similar fraction of “unidentifiable” cells even with 40-60 plex CyCIF imaging, see e.g., Lin, J. R. et al. Multiplexed 3D atlas of state transitions and immune interactions in colorectal cancer. bioRxiv 2021.03.31.437984 (2021) doi: 10.1101/2021.03.31.437984, was observed. It was surmised that these cells are either negative for all antibody markers included in the panel or have morphologies that are difficult to segment, see e.g., Yapp, C. et al. UnMICST: Deep learning with real augmentation for robust segmentation of highly multiplexed images of human tissues. 2021.04.02.438285 Preprint at https://doi.org/10.1101/2021.04.02.438285 (2022). Therefore, a previously published ML model trained on H&E data, see e.g., Kather, J. N. et al. Predicting survival from colorectal cancer histology slides using deep learning: A retrospective multicenter study. PLOS Medicine 16, e1002730 (2019), was used to identify those cells missing labels in Orion IF images and found that >50% were predicted to be smooth muscle, stromal fibroblasts, or adipocytes (FIG. 3D). These predictions were confirmed by visual inspection of the H&E images by pathologists (FIG. 3E).

Specimens (e.g., from patient 26, FIG. 3F and FIG. 9C) were identified in which a region of epithelium was weakly stained by pan-cytokeratin, E-cadherin, and immune markers making the cells difficult to identify by IF. Inspection of H&E images showed that these cells corresponded to a serrated adenoma whereas nearby invasive low-grade adenocarcinoma cells stained strongly for pan-cytokeratin and E-cadherin. Differential staining of cytokeratin isoforms in serrated adenoma and adenocarcinoma has been described previously, see e.g., Tatsumi, N. et al. Expression of Cytokeratins 7 and 20 in Serrated Adenoma and Related Diseases. Dig Dis Sci 50, 1741-1746 (2005), and in specimen C26 and likely reflects clonal heterogeneity. According to the present disclosure, the availability of H&E and IF images of the same set of cells substantially increases the fraction of cell types and states that can be identified as compared to either type of data alone. This is particularly true of cells for which specific molecular markers do not exist (e.g., stromal fibroblasts) or are lost due to tumor sub-clonality, see e.g., Lin, J. R. et al. Multiplexed 3D atlas of state transitions and immune interactions in colorectal cancer. bioRxiv 2021.03.31.437984 (2021) doi: 10.1101/2021.03.31.437984, as well as cells that are highly elongated or have multiple nuclei and are thus difficult to segment.

### EXAMPLE XVIII

### Identifying Tumor Features Predictive of Disease Progression

The classification of cancers for diagnostic purposes using American Joint Committee on Cancer (AJCC/UICC-TNM classification) criteria is based primarily on tumor-intrinsic characteristics (tumor, lymph node, and metastases, the TNM staging system), see e.g., Amin, M. B. et al. The Eighth Edition AJCC Cancer Staging Manual: Continuing to build a bridge from a population-based to a more ‘personalized’ approach to cancer staging. CA Cancer J Clin 67, 93-99 (2017). However, the extent and type of immune infiltration also plays a major role in therapeutic response and survival, see e.g., Paijens, S. T., Vledder, A., de Bruyn, M. & Nijman, H. W. Tumor-infiltrating lymphocytes in the immunotherapy era. Cell Mol Immunol 18, 842-859 (2021). In colorectal cancer (CRC) this has given rise to a clinical test, the Immunoscore®, see e.g., Galon, J. et al. Towards the introduction of the ‘Immunoscore’ in the classification of malignant tumours. The Journal of Pathology 232, 199-209 (2014), that is predictive of disease progression in multicenter cohort studies (as measured by progression-free survival, PFS, or overall survival, OS) and of time to recurrence in stage III cancers in a Phase 3 clinical trial, see e.g., Pagès, F., Taieb, J., Laurent-Puig, P. & Galon, J. The consensus Immunoscore in phase 3 clinical trials; potential impact on patient management decisions. Oncoimmunology 9, 1812221. The Immunoscore uses IHC to evaluate the number of CD3 and CD8-positive T cells at the tumor center (CT) and the invasive margin (IM; for Immunoscore this is defined as a region encompassing 360 μm on either side of the invasive boundary). See e.g., Angell, H. K., Bruni, D., Barrett, J. C., Herbst, R. & Galon, J. The Immunoscore: Colon Cancer and Beyond. Clin Cancer Res 26, 332-339 (2020); Galon, J. et al. Type, Density, and Location of Immune Cells Within Human Colorectal Tumors Predict Clinical Outcome. Science 313, 1960-1964 (2006). In the present disclosure, the invasive margin was taken to be ±100 μm from the boundary. The hazard ratio (HR; the difference in the rate of progression) between patients with tumors containing few immune cells in both the CT and the IM (Immunoscore=0) and patients with tumors containing many cells in both compartments (Immunoscore=4) has been reported to be 0.20, (95% CI 0.10-0.38; p<10-4) in a Cox regression model, with increasing score correlating with longer survival, see e.g., Pagès, F. et al. International validation of the consensus Immunoscore for the classification of colon cancer: a prognostic and accuracy study. The Lancet 391, 2128-2139 (2018). This is a clinically significant difference that can be used to inform key treatment decision: for example, whether or not to deliver chemotherapy following surgery (i.e., adjuvant therapy), see e.g., Argilés, G. et al. Localised colon cancer: ESMO Clinical Practice Guidelines for diagnosis, treatment and follow-up†. Annals of Oncology 31, 1291-1305 (2020).

Using data generated by the ORION method described herein, an automated method was developed to recapitulate key aspects of the Immunoscore using PFS as measure of survival and generating Image Feature Models. First, the tumor-stromal interface was detected and masks were generated that matched the criteria for CT and IM (+100 μm around the tumor boundary; FIG. 4A). CD3 and CD8 positivity in single cells was determined by Gaussian Mixture Modeling, see e.g., Pan, K., Kokaram, A., Hillebrand, J. & Ramaswami, M. Gaussian mixtures for intensity modeling of spots in microscopy. in 121-124 (2010). doi: 10.1109/ISBI.2010.5490398, with the median positive fraction for each marker (CD3 or CD8) in each region (CT or IM) across all 40 colorectal cancer cases used as the cutoff for assigning a subscore of 0 or 1. The sum of the four subscores was used as the final score for Image Feature Model 1 (IFM1; FIG. 4B). The scoring method was intentionally simplified to avoid a need for tuning of adjustable parameters but nonetheless yielded a hazard ratio HR=0.209 (95% CI 0.094-0.465; p=10-4) (FIG. 4C), similar to Immunoscore itself.

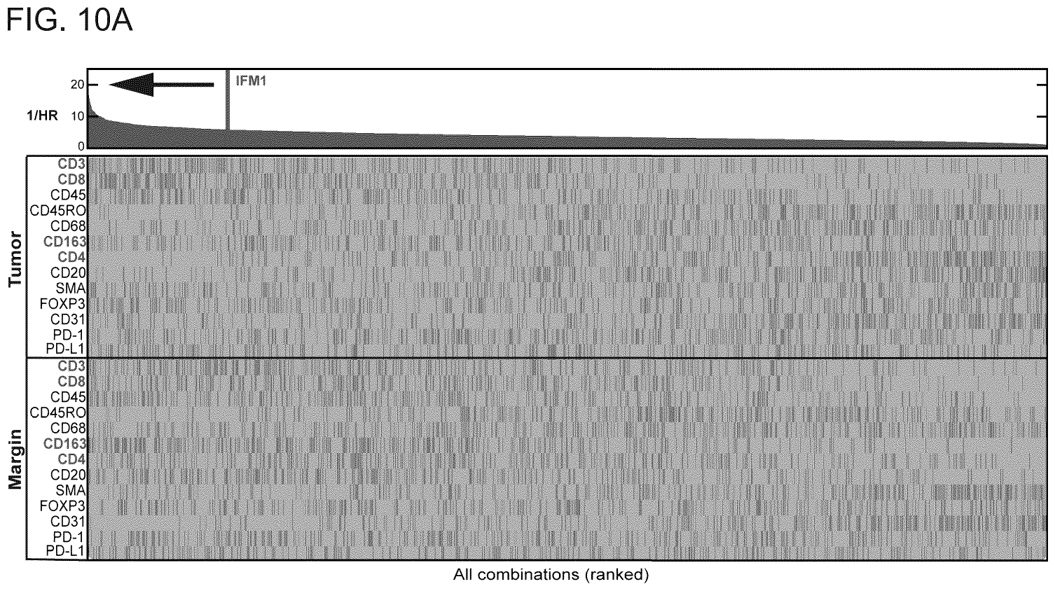

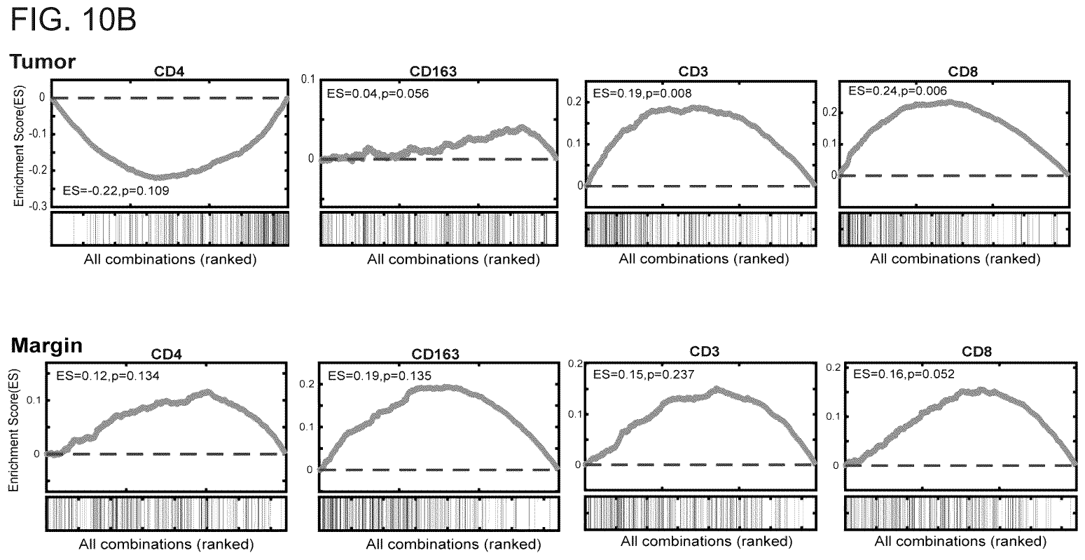

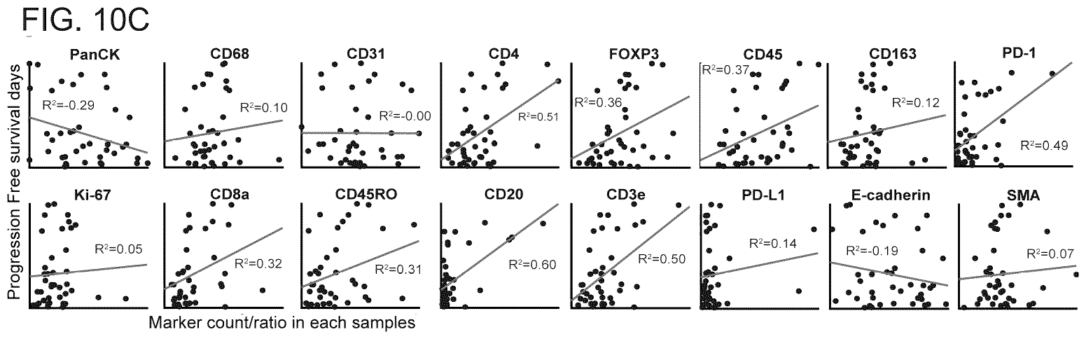

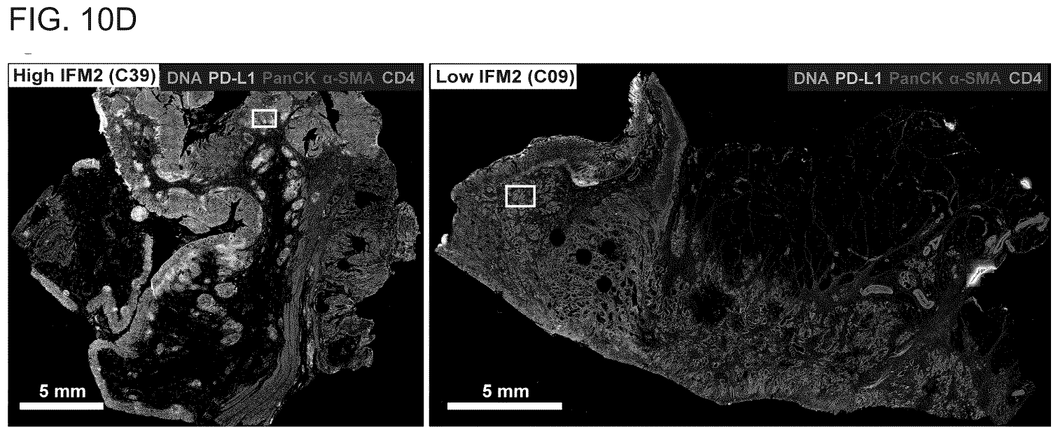

Next, a total of 13 immune focused markers were used to generate ˜15,000 marker combinations (Image Feature Models, IFMs), each composed of four markers within the tumor center (CT) and invasive margin (IM) domains (FIG. 4D). Scores for each colorectal cancer case were binarized into high and low scores based on median intensities. When hazard rations were calculated, nearly 2,500 Image Feature Models exceeded IFM1 in performance (FIGS. 10A, 10B, and 10C). The optimal model (IFM2) exhibited a hazard ratio=0.0785 (95% CI: 0.036-0.172, p=2×10-06) (FIGS. 4D and 4E) and comprised the fractions of α-SMA+ cells in the tumor center CT, and CD45+, PD-L1+ and CD4+ cells in the invasive margin IM. Leave-one-out resampling showed that IFM2 was significantly better than IFM1 and demonstrated stable ranking with respect to hazard ratio (p=3.4×10-14; adjusted p value based on the Benjamini-Hochberg Procedure padj=5.01×10-9). 500-fold bootstrapping also confirmed a distribution of hazard ratios for IFM2 that was significantly lower than for IFM1 (FIG. 4F).

Histologic review of H&E images showed that IFM2-high tumors that exhibited slow progression (e.g., patient C34) had extensive lymphohistiocytic chronic inflammation including large lymphoid aggregates and tertiary lymphoid structures (TLS) at the tumor invasive margin (so-called “Crohn's-like lymphoid reaction”), see e.g., Graham, D. M. & Appelman, H. D. Crohn's-like lymphoid reaction and colorectal carcinoma: a potential histologic prognosticator. Mod Pathol 3, 332-335 (1990), whereas IFM2-low tumors had relatively few lymphoid aggregates and no tertiary lymphoid structures TLS (e.g., patient C09) (FIG. 4G and FIG. 10D). IFM2-low tumors were also more invasive than IFM2-high tumors but scoring was independent of histologic subtypes (e.g., conventional vs. mucinous morphology) and did not correlate with histologic grade (low vs. high grade carcinoma). Thus, IFM2 is likely to capture hyperactivity of the immune microenvironment around the invasive tumor margin and potential inactivation of tumor-associated fibroblasts. The data obtained from the ORION method can be used to automate previously described image-based biomarkers based on singlechannel IHC and identify new marker combinations that significantly outperform them.

### EXAMPLE XIX

### Identifying New Progression Markers

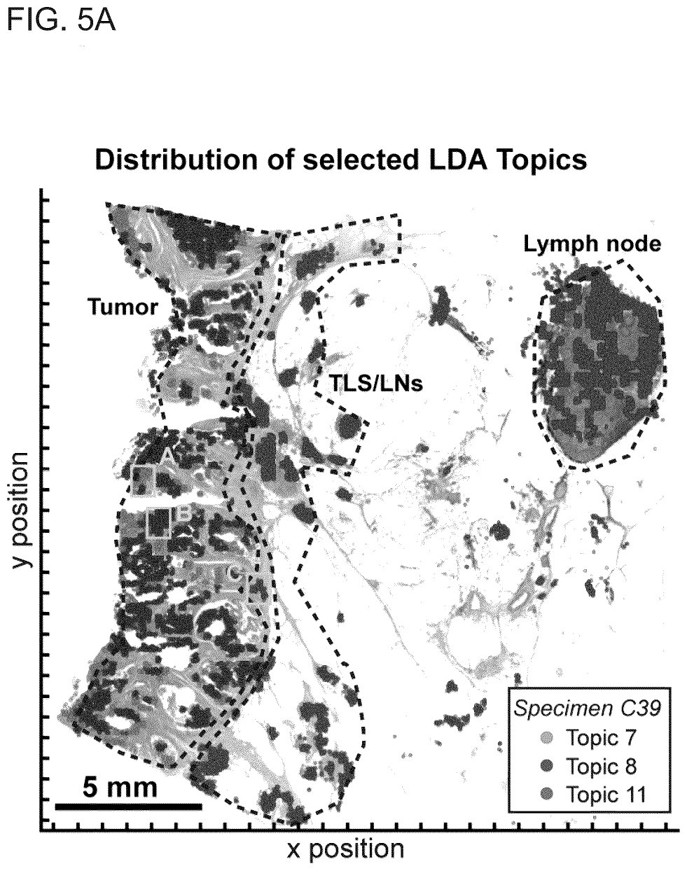

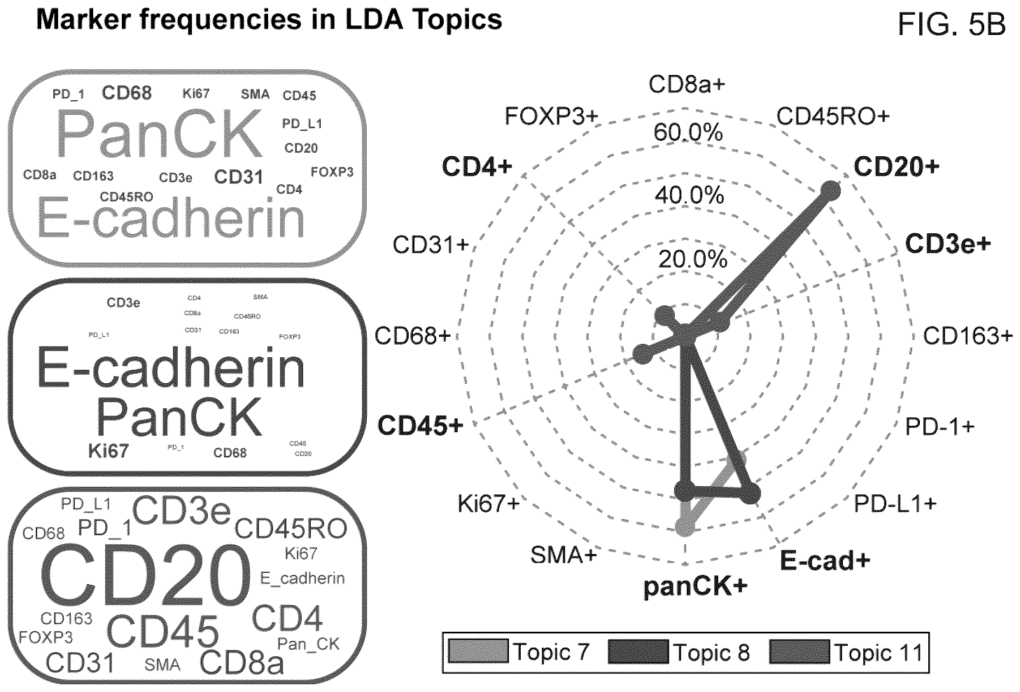

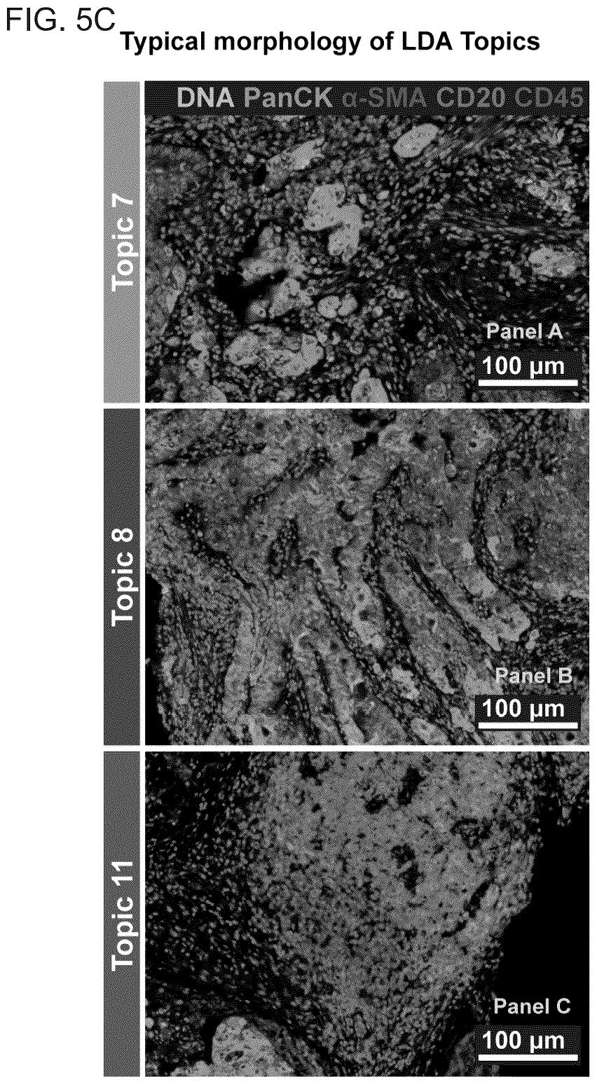

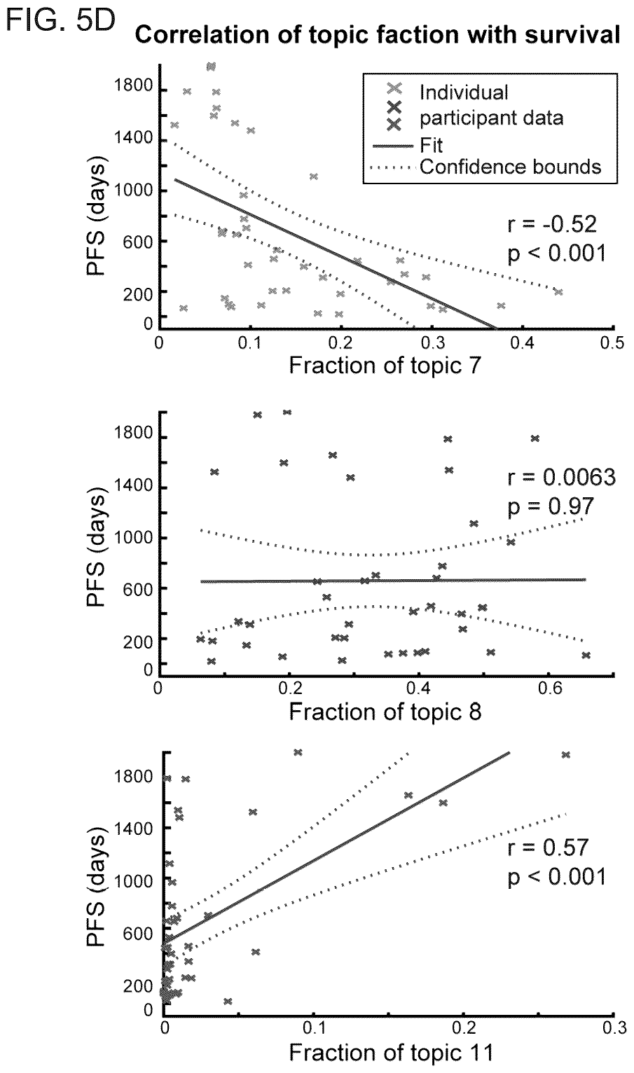

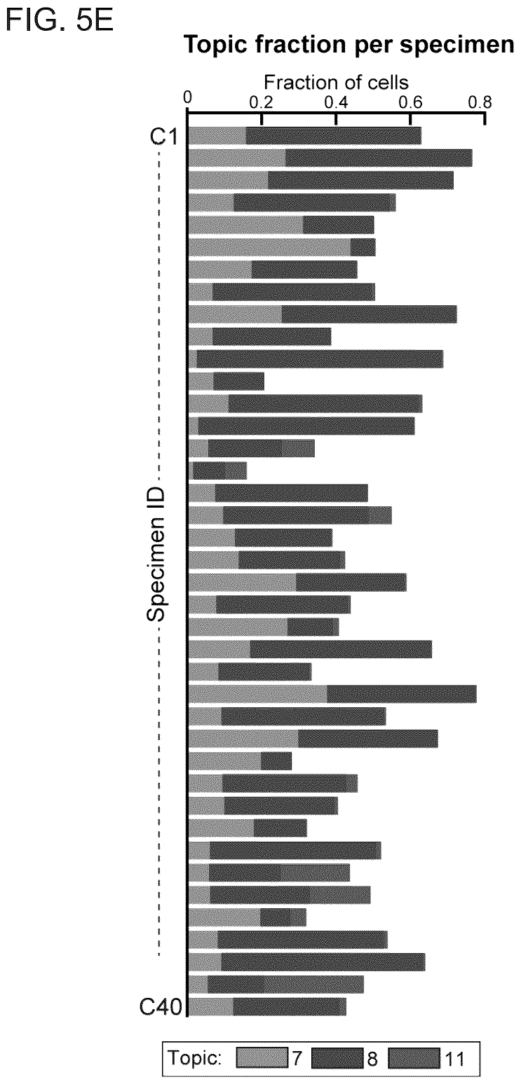

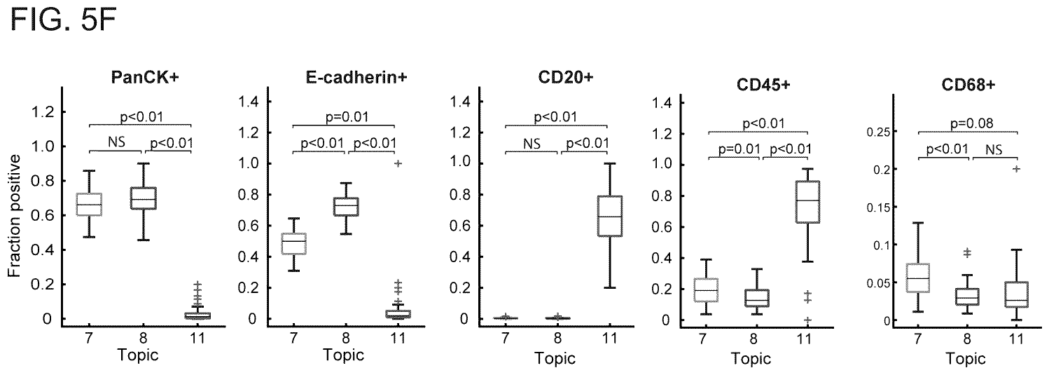

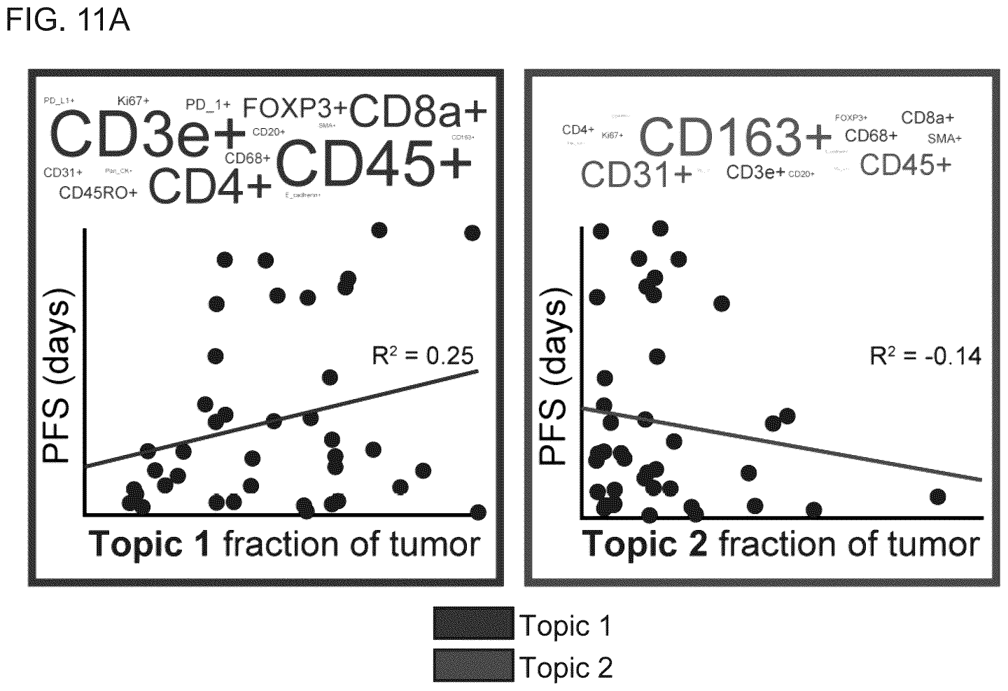

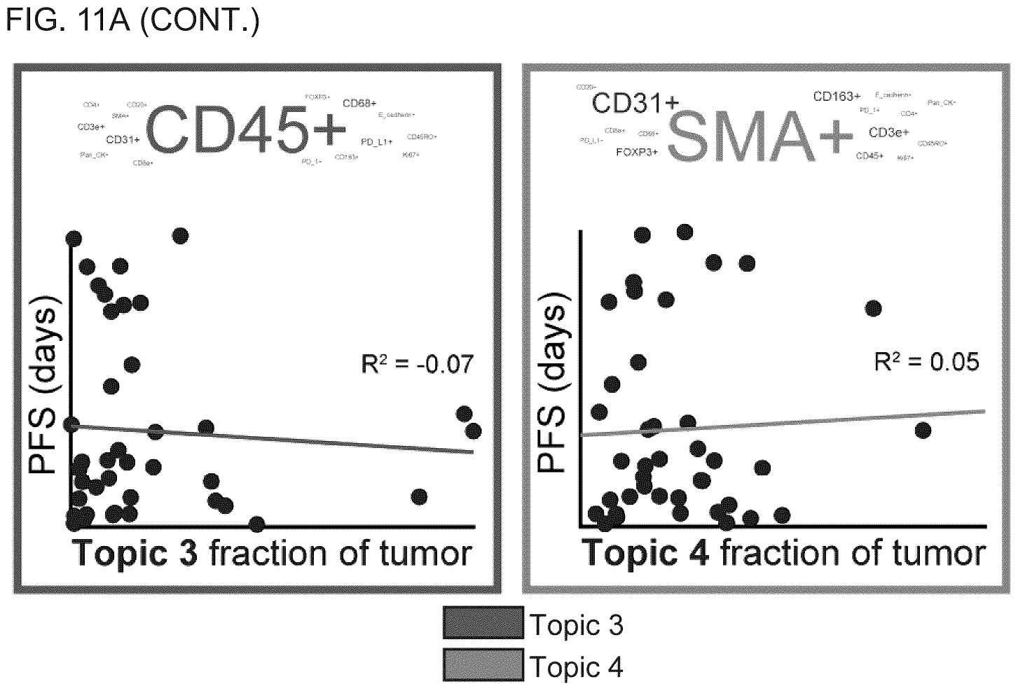

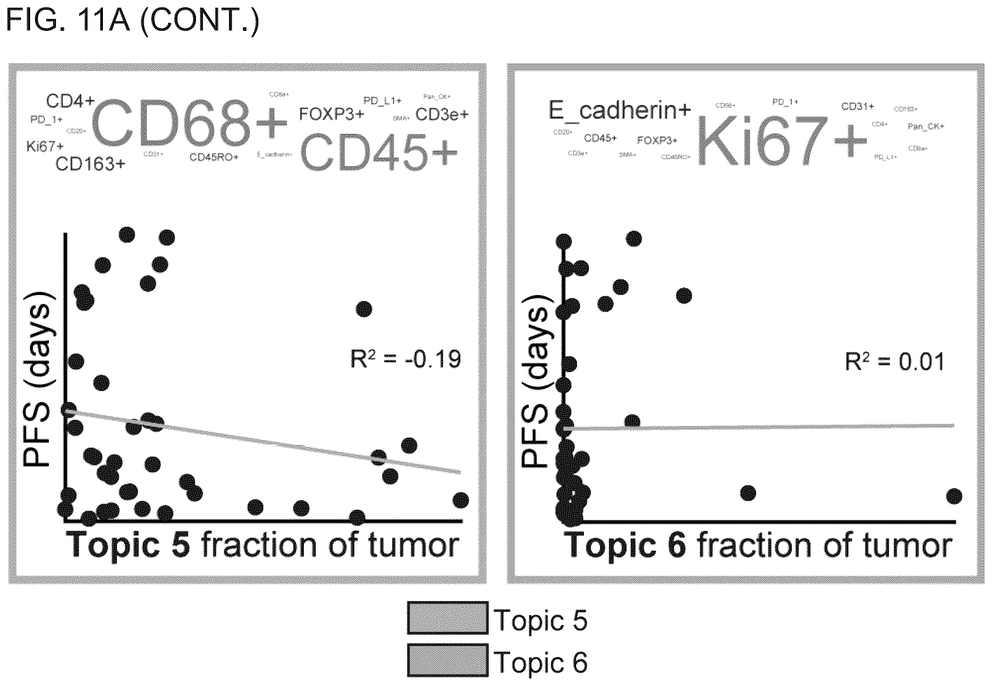

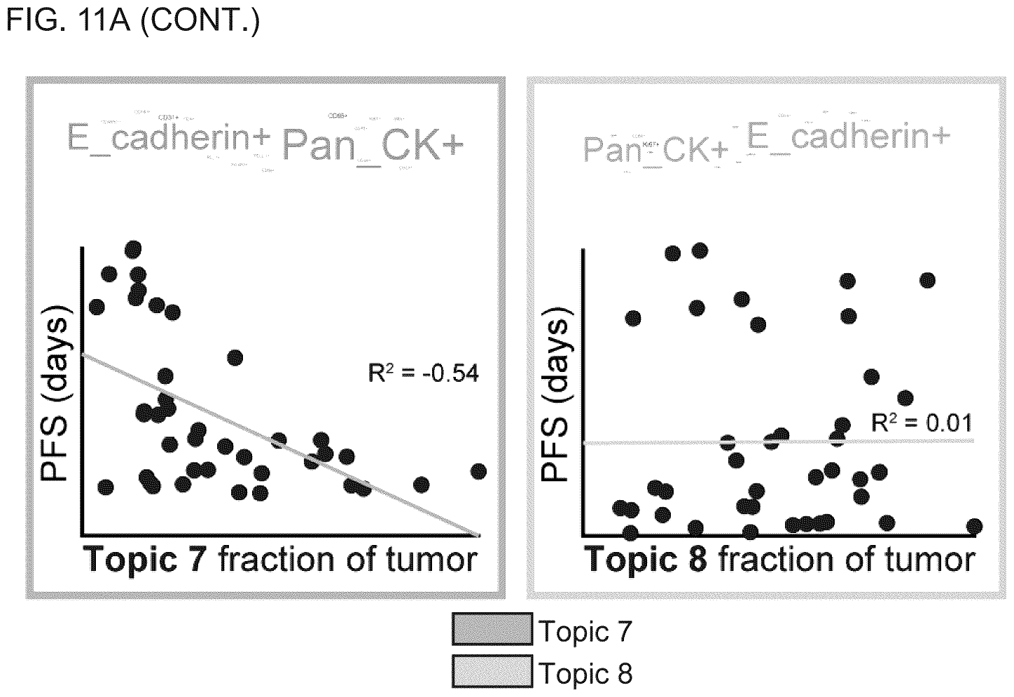

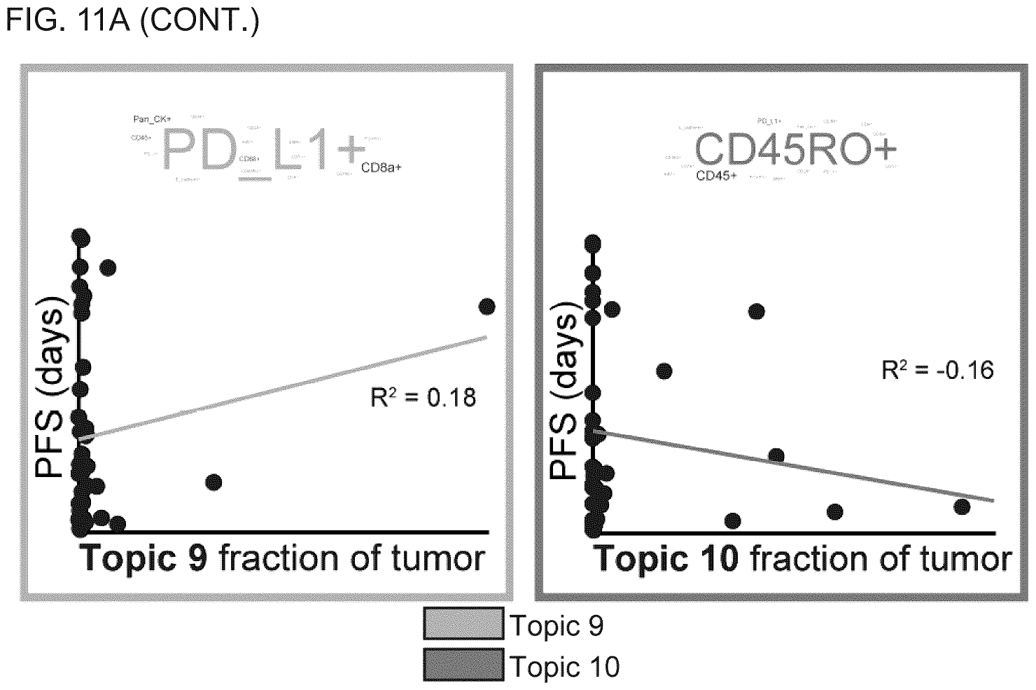

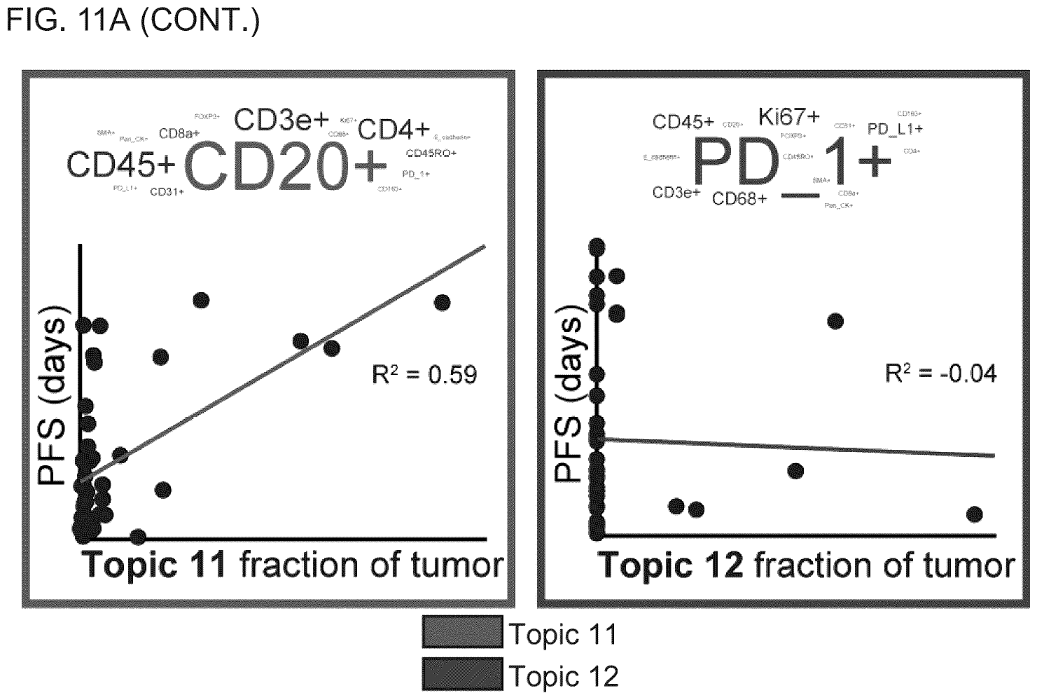

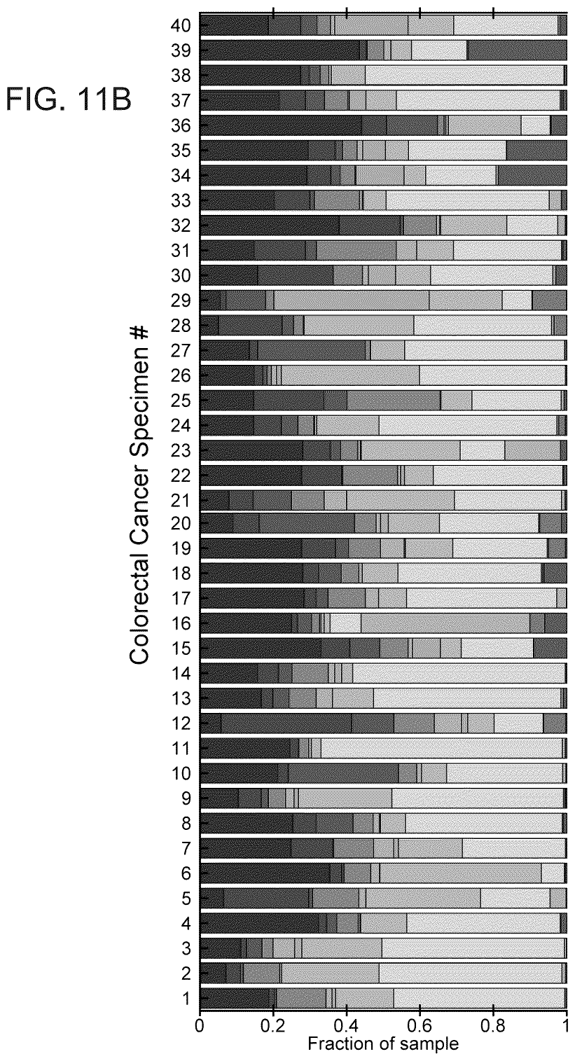

As an unbiased bottom-up means of identifying new progression models, the machine learning tool Latent Dirichlet Allocation (LDA), see e.g., Blei, D. M., Ng, A. Y. & Jordan, M. I. Latent dirichlet allocation. J. Mach. Learn. Res. 3, 993-1022 (2003), was used, which is a probabilistic modeling method that reduces complex assemblies of intermixed entities into distinct component communities (“topics”). LDA is widely used in text mining and biodiversity studies and can detect recurrent arrangements of words or natural elements while accounting for uncertainty and missing data, see e.g., Valle, D., Baiser, B., Woodall, C. W. & Chazdon, R. Decomposing biodiversity data using the Latent Dirichlet Allocation model, a probabilistic multivariate statistical method. Ecology Letters 17, 1591-1601 (2014); Jackson, H. W. et al. The single-cell pathology landscape of breast cancer. Nature 578, 615-620 (2020). Colorectal cancer specimens were separated into tumor and adjacent normal tissue using H&E data and an ML/AI model44 and Latent Dirichlet Allocation LDA was performed at the level of individual IF markers on cells in the tumor region (FIG. 5A). This yielded 12 spatial features (topics) that recurred across the dataset (the number of topics was optimized by calculating the perplexity) (FIG. 11A).

Visual inspection of images by a pathologist confirmed that marker probabilities matched those computed for different topics and that the frequency distribution of each topic varied, sometimes substantially, among colorectal cancer samples (FIG. 5B and FIG. 11A-11B). The strongest correlations between topics and progression free survival PFS for the 40 colorectal cancer cohort were found to be −0.52 (p<0.001) for Topic 7, comprising pan-cytokeratin and E-cadherin positivity, with little contribution from immune cells, and +0.57 (p<0.001) for Topic 11, comprising CD20 positivity with minor contributions from CD3, CD4, and CD45 (FIG. 5B-5F and FIG. 11A-11B). In contrast, topics involving the proliferation marker Ki-67+ (Topic 6), PD-L1 positivity (Topic 9), or immune cells markers (CD45+ or CD45RO+; Topics 3 and 10) exhibited little or no correlation with survival (FIG. 11A-11B).

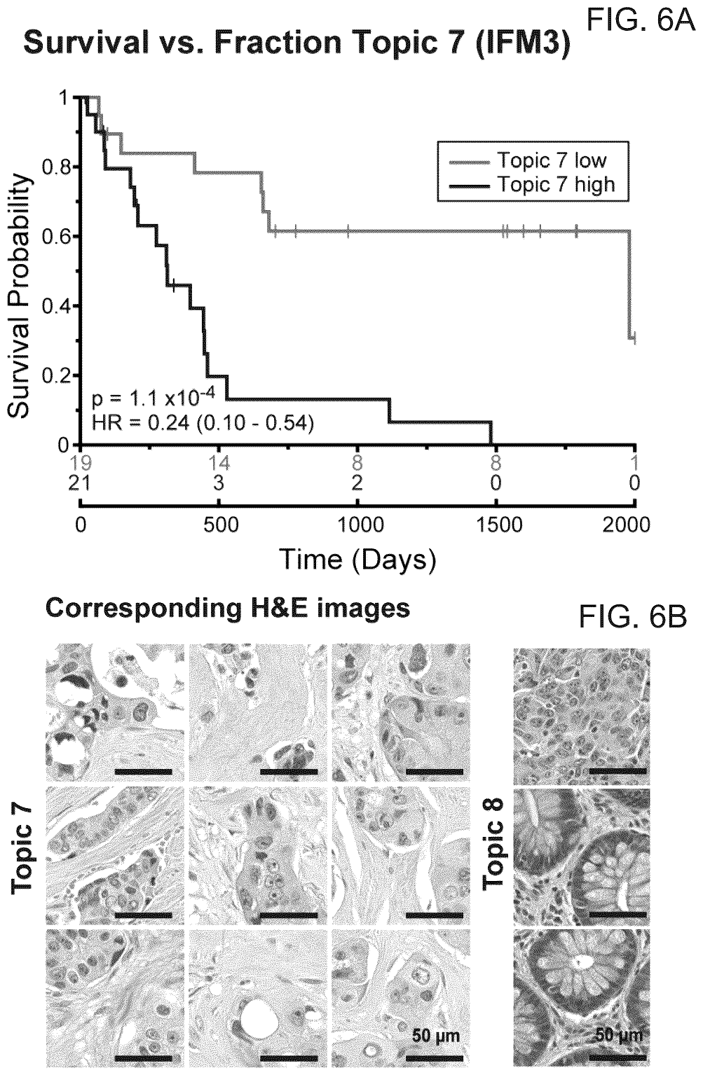

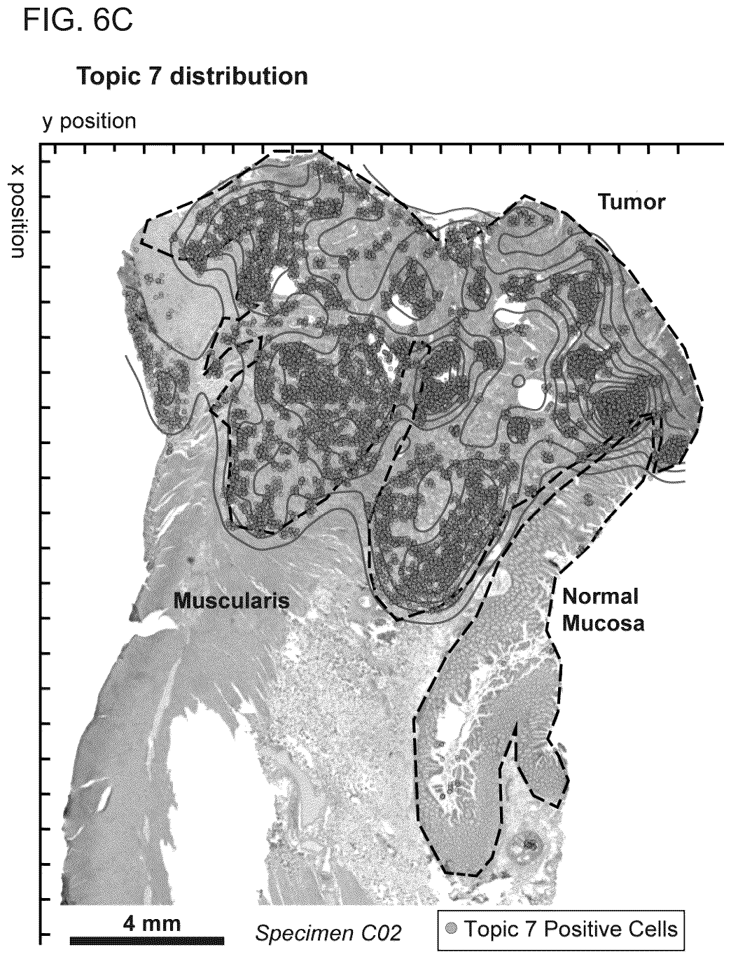

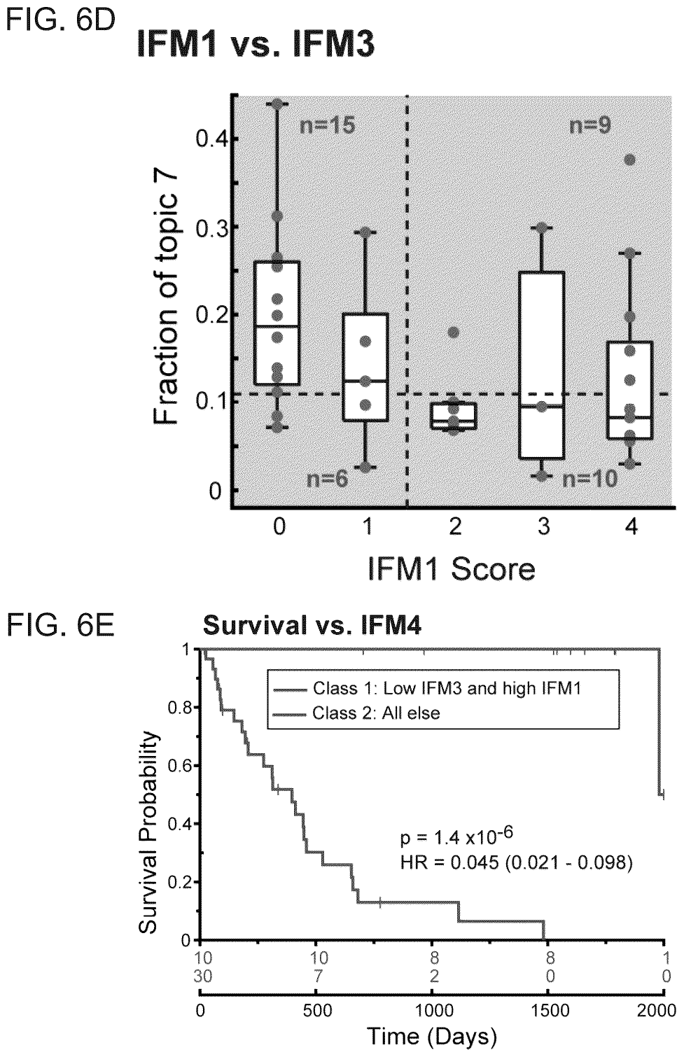

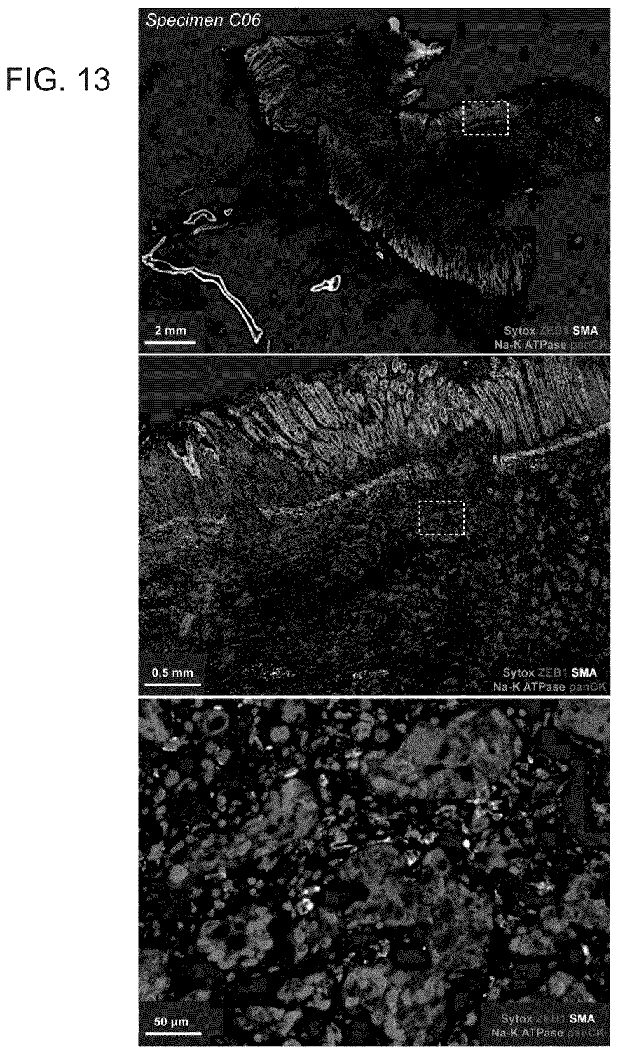

Given the correlation of Topic 7 with progression free survival, a Kaplan-Meier curve was constructed for tumors having a proportion of Topic 7 below the 25th percentile versus those above this threshold (including all cells in the specimen) and observed a hazard ratio HR=0.24 (FIG. 6A; CI 95%: 0.10-0.54; p<10-3). Thus, the machine learning tool Latent Dirichlet Allocation was successfully used to identify-via direct analysis of high-plex IF data-a tumor-intrinsic feature distinct from immune infiltration that was significantly associated with poor patient survival. In the case of Topic 7, the primary molecular features were pan-cytokeratin and E-cadherin positivity, but Topic 8 was similar in composition while exhibiting no correlation with PFS (r=0.01; FIG. 5C and FIG. 11A-11B). To identify the tumor histomorphology corresponding to Topic 7 and Topic 8, labels from IF were transferred to the same section H&E images, trained a convolutional neural network (CNN) on the H&E data, and inspected the highest scoring tumor regions (FIG. 12A). In the case of Topic 7, these were readily identifiable as regions of poorly differentiated/high-grade tumor with stromal invasion (FIGS. 6A and 6B). In contrast, Topic 8 consisted predominantly of intestinal mucosa with a largely normal morphology and some areas of well-differentiated tumor (FIG. 6B and FIG. 12B). Inspection of Orion and CyCIF images of specimens with a high proportion of Topic 7 (e.g., patient C06, FIG. 13) revealed that the E-cadherin to pan-cytokeratin ratios were low relative to normal mucosa or Topic 8 (Na, K-ATPase expression was also low). These features of cells undergoing an epithelial-mesenchymal transition (EMT) are associated in colorectal cancer with progression and metastasis, see e.g., Cao, H., Xu, E., Liu, H., Wan, L. & Lai, M. Epithelial-mesenchymal transition in colorectal cancer metastasis: A system review. Pathol Res Pract 211, 557-569 (2015). However, some features of epithelial-mesenchymal transition EMT were not observed in Topic 7-positive cells: proliferation index was high (40-50% Ki67 and PCNA positivity) and staining for the EMT marker and transcription factor ZEB1 was low (when assessed using CyCIF data) 60. Thus, even though the molecular and morphological features of Topic 7 were consistent with each other, H&E morphology was more readily interpretable with respect to long established features of colorectal cancer progression. It has been observed previously that interpretability increases confidence in a potential biomarker and substantially improves its chances of clinical translation, see e.g., Ludwig, J. A. & Weinstein, J. N. Biomarkers in cancer staging, prognosis and treatment selection. Nat Rev Cancer 5, 845-856 (2005).

Only about one-third of patients scored high for IFM1 and low for IFM3 (the combination correlated with the longest PFS; FIG. 6D). According to one aspect, the two models were combined. Using the composite model (IFM4), near perfect discrimination was observed between progressing and non-progressing colorectal cancer patients with hazard ratio HR=0.045; (95% CI=0.021 to 0.098; p=1.4×10-6) (FIG. 6E). This demonstrates that immunological and tumor-intrinsic features of cancers arising from top-down and bottom-up analysis can be effectively combined to generate prognostic models with high predictive value. Of note, no parameter tuning (e.g., setting thresholds for positivity) was involved in the generation of IFMs 1-3 or the highly performative IFM4 hybrid model. Experience with Immunoscore shows that parameter tuning using larger cohorts of patients can further boost performance.

### EXAMPLE XX

### Colorectal Cancer Treatment

According to certain aspects, colorectal cancer is treated, if desired, by methods known to those of skill in the art. According to one aspect, different types of doctors may work together as a multidisciplinary team to create a patient's overall treatment plan that usually includes or combines different types of treatments. For colorectal cancer, this may include a surgeon, medical oncologist, radiation oncologist, and a gastroenterologist. Cancer care teams include a variety of other health care professionals, such as physician assistants, nurse practitioners, oncology nurses, social workers, pharmacists, counselors, dietitians, and others.

Treatment options and recommendations depend on several factors, including the type and stage of cancer, possible side effects, the patient's medical condition, the patient's preferences and overall health. Common treatment options include surgery, radiation therapy, chemotherapy, targeted therapy, and immunotherapy.

Surgery is the removal of the tumor and some surrounding healthy tissue during an operation. It is often called surgical resection. This is the most common treatment for colorectal cancer. Part of the healthy colon or rectum and nearby lymph nodes may also be removed.

Radiation therapy is the use of high-energy x-rays to destroy cancer cells. It is commonly used for treating rectal cancer because this kind of tumor tends to recur near where it originally started. A radiation therapy regimen, or schedule, usually consists of a specific number of treatments given over a set period of time. External-beam radiation therapy uses a machine to deliver x-rays to where the cancer is located. Radiation treatment is usually given 5 days a week for several weeks. It may be given in the doctor's office or at the hospital. Stereotactic radiation therapy is a type of external-beam radiation therapy that may be used if colorectal cancer has spread to the liver or lungs. This type of radiation therapy delivers a large, precise radiation dose to a small area. This technique can help save parts of the liver and lung tissue that might otherwise have to be removed during surgery. However, not all cancers that have spread to the liver or lungs can be treated in this way. For some people, specialized radiation therapy techniques, such as intraoperative radiation therapy or brachytherapy, may help get rid of small areas of cancer that can not be removed with surgery. Intraoperative radiation therapy uses a single, high dose of radiation therapy given during surgery. Brachytherapy is the use of radioactive “seeds” placed inside the body. For rectal cancer, radiation therapy may be used before surgery, called neoadjuvant therapy, to shrink the tumor so that it is easier to remove. It may also be used after surgery to destroy any remaining cancer cells. Chemotherapy is often given at the same time as radiation therapy to increase the effectiveness of the radiation therapy. This is called chemoradiation therapy.

Treatment may include medications to destroy cancer cells. Medication may be given through the bloodstream to reach cancer cells throughout the body. When a drug is given this way, it is called systemic therapy. Medication may also be given locally, which is when the medication is applied directly to the cancer or kept in a single part of the body. This type of medication is generally prescribed by a medical oncologist, a doctor who specializes in treating cancer with medication. Medications are often given through an intravenous (IV) tube placed into a vein using a needle or as a pill or capsule that is swallowed (orally). The types of medications used for colorectal cancer include: chemotherapy; targeted therapy; and immunotherapy. Each can be used separately or in combination and can also be given as part of a treatment plan that includes surgery and/or radiation therapy. Chemotherapy is the use of drugs to destroy cancer cells, usually by keeping the cancer cells from growing, dividing, and making more cells. A chemotherapy regimen, or schedule, usually consists of a specific number of cycles given over a set period of time. A patient may receive 1 drug at a time or a combination of different drugs given at the same time. Chemotherapy may be given after surgery to eliminate any remaining cancer cells. For some people with rectal cancer, the doctor will give chemotherapy and radiation therapy before surgery to reduce the size of a rectal tumor and reduce the chance of the cancer returning. Many drugs are approved by the U.S. Food and Drug Administration (FDA) to treat colorectal cancer in the United States and include Capecitabine (Xeloda); Fluorouracil (5-FU); Irinotecan (Camptosar); Oxaliplatin (Eloxatin); and Trifluridine/tipiracil (Lonsurf). These drugs can be used alone or in combination.

Targeted therapy is a treatment that targets the cancer's specific genes, proteins, or the tissue environment that contributes to cancer growth and survival. This type of treatment blocks the growth and spread of cancer cells and limits damage to healthy cells. Not all tumors have the same targets. Accordingly, tests may be run to identify the genes, proteins, and other factors associate with colorectal cancer. For colorectal cancer, the following targeted therapies may be options. Anti-angiogenesis therapy is focused on stopping angiogenesis, which is the process of making new blood vessels. Because a tumor needs the nutrients delivered by blood vessels to grow and spread, the goal of anti-angiogenesis therapies is to “starve” the tumor. Exemplary drugs include Bevacizumab (Avastin); Regorafenib (Stivarga); and Ziv-aflibercept (Zaltrap) and ramucirumab (Cyramza). Epidermal growth factor receptor (EGFR) inhibitors may be effective for stopping or slowing the growth of colorectal cancer. Exemplary drugs include Cetuximab (Erbitux); and Panitumumab (Vectibix). Some tumors have a specific mutation, called BRAF V600E, that can be detected by an FDA-approved test. A class of targeted treatments called BRAF inhibitors can be used to treat tumors with this mutation. A combination using the BRAF inhibitor encorafenib (Braftovi) and cetuximab may be used to treat people with metastatic colorectal cancer with this mutation who have received at least 1 previous treatment. Some tumors express a protein called HER2 that can be targeted by specific medications. If this happens, the cancer is called HER2-positive. For people with HER2-positive advanced colorectal cancer, treatment with a combination of tucatinib (Tukysa) and trastuzumab (Herceptin and other brand names) may be an option. This combination may only be used if there are no mutations in the RAS gene, surgery is not an option, and chemotherapy has stopped working and/or caused side effects that require stopping treatment. Larotrectinib (Vitrakvi) and entrectinib (Rozlytrek) are types of targeted therapy that are not specific to a certain type of cancer but focus on a specific genetic change called an NTRK fusion. This type of genetic change is rare but is found in a range of cancers, including colorectal cancer. These medications are approved as treatment for colorectal cancer that is metastatic or cannot be removed with surgery and has worsened with other treatments.

Immunotherapy uses the body's natural defenses to fight cancer by improving the immune system's ability to attack cancer cells. Checkpoint inhibitors are exemplary of immunotherapy agents used to treat colorectal cancer. Pembrolizumab targets PD-1, a receptor on tumor cells, preventing the tumor cells from hiding from the immune system. Pembrolizumab is used to treat unresectable or metastatic colorectal cancers that have a molecular feature called microsatellite instability (MSI-H) or mismatch repair deficiency (dMMR). Unresectable means surgery is not an option. Nivolumab is used to treat people who are 12 or older and have MSI-H or dMMR metastatic colorectal cancer that has grown or spread after treatment with chemotherapy with a fluoropyrimidine (such as capecitabine and fluorouracil), oxaliplatin, and irinotecan. Dostarlimab is a PD-1 immune checkpoint inhibitor. It may be used to treat recurrent or metastatic colorectal cancers that have dMMR. Nivolumab and ipilimumab (Yervoy) is a combination of checkpoint inhibitors approved to treat patients who are 12 or older and have MSI-H or dMMR metastatic colorectal cancer that has grown or spread after treatment with chemotherapy with a fluoropyrimidine, oxaliplatin, and irinotecan.

Colorectal cancer can be treated according to the stage of colorectal cancer. In general, stages 0, I, II, and III are often curable with surgery. However, many people with stage III colorectal cancer, and some with stage II, receive chemotherapy after surgery to increase the chance of eliminating the disease. People with stage II and III rectal cancer will also receive radiation therapy with chemotherapy either before or after surgery. Stage IV is not often curable, but it is treatable, and the growth of the cancer and the symptoms of the disease can be managed.

For Stage 0 colorectal cancer, the usual treatment is a polypectomy, or removal of a polyp, during a colonoscopy. There is no additional surgery unless the polyp cannot be fully removed.

For Stage I colorectal cancer, surgical removal of the tumor and lymph nodes is usually the only treatment needed.

For Stage II colorectal cancer, surgery is often the first treatment. People with stage II colorectal cancer may received adjuvant chemotherapy. Adjuvant chemotherapy is treatment after surgery with the goal of trying to destroy any remaining cancer cells.

For Stage III colorectal cancer, treatment usually involves surgical removal of the tumor followed by adjuvant chemotherapy. A clinical trial may also an option. The duration of adjuvant therapy depends on the risk of recurrence (based on characteristics of the cancer that was removed at surgery).

For Stage IV metastatic colorectal cancer, treatment may include a combination of chemotherapy, targeted therapy, immunotherapy, surgery, and radiation therapy, as described above, which can be used to slow the spread of the disease and shrink a cancerous tumor. Palliative care will also be important to help relieve symptoms of cancer and side effects of treatment.

### EXAMPLE XXI

### Computer Systems Used to Implements Aspects of the Disclosed Methods

One or more computer systems may be used to implements one or more steps or aspects of the disclosed methods. Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components.

A computer system can include a plurality of the same components or subsystems, e.g., connected together by external interface or by an internal interface. In some embodiments, computer systems, subsystems, or apparatuses can communicate over a network. In such instances, one computer can be considered a client and another computer a server, where each can be part of a same computer system. A client and a server can each include multiple systems, subsystems, or components.

It should be understood that any of the embodiments of the present invention can be implemented in the form of control logic using hardware (e.g. an application specific integrated circuit or field programmable gate array) and/or using computer software with a generally programmable processor in a modular or integrated manner. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer program product (e.g. a hard drive, a CD, or an entire computer system), and may be present on or within different computer program products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The following references are incorporated by reference within the specification in accordance with the corresponding superscript numbers as if fully set forth therein.

1. A method comprising
determining presence of pan-cytokeratin and E-cadherin in tumor cells and nontumor cells of a colorectal tissue sample obtained from a human patient by (i) contacting pan-cytokeratin with an antibody to pan-cytokeratin and detecting binding between pan-cytokeratin and the antibody to pan-cytokeratin and (ii) contacting E-cadherin with an antibody to E-cadherin and detecting binding between E-cadherin and the antibody to E-cadherin,
determining levels of pan-cytokeratin and E-cadherin in the nontumor cells of the colorectal tissue sample and determining a ratio of pan-cytokeratin and E-cadherin in the nontumor cells of the colorectal tissue sample to establish a nontumor cell control baseline ratio,
determining levels of pan-cytokeratin and E-cadherin in the tumor cells of the colorectal tissue sample and determining a ratio of pan-cytokeratin and E-cadherin in the tumor cells of the colorectal tissue sample to establish a tumor cell ratio, and
comparing the tumor cell ratio to the nontumor cell control baseline ratio.

2. The method of claim 1 wherein a tumor cell ratio that is greater than the nontumor cell control baseline ratio indicates that the tumor cells are progressive colorectal cancer tumor cells.

3. The method of claim 1 wherein a tumor cell ratio that is substantially similar to the nontumor cell control baseline ratio indicates that the tumor cells are nonprogressive colorectal cancer tumor cells.

4. The method of claim 1 wherein the colorectal tissue sample is a whole slide colorectal tissue sample.

5. The method of claim 1 wherein the colorectal tissue sample is stained to identify tissue morphology after being contacted with the antibodies.

6. The method of claim 1 wherein a tumor cell ratio that is greater than the nontumor cell control baseline ratio indicates that the tumor cells are progressive colorectal cancer tumor cells, and
further comprising treating the patient for colorectal cancer with surgery.

7. The method of claim 1 wherein a tumor cell ratio that is greater than the nontumor cell control baseline ratio indicates that the tumor cells are progressive colorectal cancer tumor cells, and
further comprising treating the patient for colorectal cancer with chemotherapy.

8. The method of claim 1 wherein a tumor cell ratio that is greater than the nontumor cell control baseline ratio indicates that the tumor cells are progressive colorectal cancer tumor cells, and
further comprising treating the patient for colorectal cancer with radiation therapy.

9. The method of claim 1 wherein a tumor cell ratio that is greater than the nontumor cell control baseline ratio indicates that the tumor cells are progressive colorectal cancer tumor cells, and
further comprising treating the patient for colorectal cancer with immunotherapy.

10. The method of claim 1 wherein a tumor cell ratio that is greater than the nontumor cell control baseline ratio indicates that the tumor cells are progressive colorectal cancer tumor cells, and
further comprising treating the patient for colorectal cancer with adjuvant therapy.

11. The method of claim 1 wherein a tumor cell ratio that is greater than the nontumor cell control baseline ratio indicates that the tumor cells are progressive colorectal cancer tumor cells, and
further comprising treating the patient for colorectal cancer with neoadjuvant therapy.

12. The method of claim 1 wherein a low value for E-cadherin in the tumor cells compared to the nontumor cells indicates that the tumor cells are progressive colorectal cancer tumor cells.

13. The method of claim 1 wherein a ratio of pan-cytokeratin to E-cadherin in tumor cells is determined normalized to a ratio of pan-cytokeratin to E-cadherin in nontumor cells, and the ratio of pan-cytokeratin to E-cadherin in tumor cells is greater than 1.

14. The method of claim 1 wherein a ratio of E-cadherin to pan-cytokeratin in tumor cells is determined normalized to a ratio of E-cadherin to pan-cytokeratin in nontumor cells, and the ratio of pan-cytokeratin to E-cadherin in tumor cells is less than 1.

15. A method of distinguishing progressive cancer cells from nonprogressive cancer cells in a colorectal tissue sample from a human patient comprising
determining presence of pan-cytokeratin and E-cadherin in tumor cells and nontumor cells of the colorectal tissue sample by (i) contacting pan-cytokeratin with an antibody to pan-cytokeratin and detecting binding between pan-cytokeratin and the antibody to pan-cytokeratin and (ii) contacting E-cadherin with an antibody to E-cadherin and detecting binding between E-cadherin and the antibody to E-cadherin,
determining levels of pan-cytokeratin and E-cadherin in the nontumor cells of the colorectal tissue sample and determining a ratio of pan-cytokeratin and E-cadherin in the nontumor cells of the colorectal tissue sample to establish a nontumor cell control baseline ratio,
determining levels of pan-cytokeratin and E-cadherin in the tumor cells of the colorectal tissue sample and determining a ratio of pan-cytokeratin and E-cadherin in the tumor cells of the colorectal tissue sample to establish a tumor cell ratio, and
comparing the tumor cell ratio to the nontumor cell control baseline ratio,
wherein a tumor cell ratio that is greater than the nontumor cell control baseline ratio indicates that the tumor cells are progressive colorectal cancer tumor cells, and
wherein a tumor cell ratio that is substantially similar to the nontumor cell control baseline ratio indicates that the tumor cells are nonprogressive colorectal cancer tumor cells.

16. The method of claim 15, further comprising
treating the patient for colorectal cancer with one or more of surgery, chemotherapy, radiation therapy, immunotherapy, adjuvant therapy or neoadjuvant therapy.

17. A method of detecting a pattern of biomarkers within a colorectal tissue sample of a human,
wherein the colorectal tissue sample comprises tumor cells and nontumor cells,
wherein the biomarkers comprise pan-cytokeratin and E-cadherin,
comprising
contacting the colorectal tissue sample with an antibody that identifies presence in the colorectal tissue sample of pan-cytokeratin,
contacting the colorectal tissue sample with an antibody that identifies presence in the colorectal tissue sample of E-cadherin,
acquiring pattern detection of the antibody that identifies presence in the colorectal tissue sample of pan-cytokeratin,
acquiring pattern detection of the antibody that identifies presence in the colorectal tissue sample of E-cadherin.

18. A method of assaying for pan-cytokeratin and E-cadherin within a colorectal tissue sample of a human, wherein the colorectal tissue sample comprises tumor cells and nontumor cells comprising
contacting the colorectal tissue sample with a first antibody that identifies presence in the colorectal tissue sample of pan-cytokeratin,
contacting the colorectal tissue sample with a second antibody that identifies presence in the colorectal tissue sample of E-cadherin,
determining amount of pan-cytokeratin relative to amount of E-cadherin within the tumor cells of the colorectal tissue sample,
determining amount of pan-cytokeratin relative to amount of E-cadherin within the nontumor cells of the colorectal tissue sample.

19. The method of claim 18, further comprising comparing (i) the amount of pan-cytokeratin relative to the amount of E-cadherin within the tumor cells of the colorectal tissue sample to (ii) the amount of pan-cytokeratin relative to the amount of E-cadherin within the nontumor cells of the colorectal tissue sample.

20. The method of claim 18 wherein the colorectal tissue sample is a whole slide colorectal tissue sample.

21. The method of claim 18 wherein the colorectal tissue sample is stained to identify tissue morphology after being contacted with the antibodies.

22. A method of treating a human for progressive colorectal cancer comprising
assaying for a ratio of pan-cytokeratin to E-cadherin within tumor cells of a colorectal tissue sample,
assaying for a ratio of pan-cytokeratin to E-cadherin within nontumor cells of the colorectal tissue sample, and
treating the human for progressive colorectal cancer when the ratio of pan-cytokeratin to E-cadherin within tumor cells of the colorectal tissue sample is higher than the ratio of pan-cytokeratin to E-cadherin within nontumor cells of the colorectal tissue sample.

23. A method of treating a human for progressive colorectal cancer comprising
assaying for a ratio of pan-cytokeratin and E-cadherin within tumor cells of a colorectal tissue sample,
assaying for a ratio of pan-cytokeratin and E-cadherin within nontumor cells of the colorectal tissue sample, and
treating the human for progressive colorectal cancer when the ratio of pan-cytokeratin and E-cadherin within tumor cells of the colorectal tissue sample is different than the ratio of pan-cytokeratin and E-cadherin within nontumor cells of the colorectal tissue sample.

24. A combination of a labeled antibody to pan-cytokeratin and a labeled antibody to E-cadherin for the staining of a colorectal tissue sample.

25. A colorectal tissue sample from a human suspected of having colorectal cancer that is stained with a labeled antibody to pan-cytokeratin and a labeled antibody to E-cadherin.

26. A kit for use in an immunofluorescence assay comprising
a first labeled antibody to pan-cytokeratin in a suitable liquid medium,
a second labeled antibody to E-cadherin in a suitable liquid medium, and
optional instructions for contacting the first labeled antibody to pan-cytokeratin and the second labeled antibody to E-cadherin to a tissue sample.

27. The kit according to claim 26 wherein
the first labeled antibody to pan-cytokeratin is in a suitable liquid medium, and
the second labeled antibody to E-cadherin is in a suitable liquid medium.

28. The kit of claim 26 wherein the first labeled antibody is in a first vessel and the second labeled antibody is in a second vessel.

29. The kit of claim 26 wherein the first labeled antibody and the second labeled antibody are combined in a vessel.
